(19) United States
(12) Patent Application Publication
(10) Pub. No.: US 2011/0223248 A1
(43) Pub. Date: Sep. 15, 2011

(54) METHODS AND COMPOSITIONS FOR TREATING LACTOSE INTOLERANCE

(75) Inventor: Andrew J. Ritter, Los Angeles, CA (US)

(73) Assignee: Ritter Pharmaceuticals, Inc., Los Angeles, CA (US)

(21) Appl. No.: 13/083,340

(22) Filed: Apr. 8, 2011

Related U.S. Application Data
(63) Continuation-in-part of application No. 12/055,936, filed on Mar. 26, 2008.
Application No. 12/055,936 is a continuation of application No. 11/632,289, filed on Dec. 12, 2007.
(60) Provisional application No. 61/322,211, filed on Apr. 8, 2010.

(51) Int. Cl. A61K 9/48; A61K 31/7016; A61K 35/74; A61K 9/28; A61P 1/00; A61P 19/10

(52) U.S. Cl. 424/463; 514/53; 424/93.45; 424/474

(57) ABSTRACT

The invention provides methods and compositions for treating symptoms associated with lactose intolerance and for overall improvement in gastrointestinal health. Described herein are methods and compositions for improving overall gastrointestinal health or for decreasing symptoms of lactose intolerance by administering to subject in need thereof a lactose composition optionally in combination with effective amount of a probiotic, prebiotic, or both.

### CROSS REFERENCE

This application claims the benefit of U.S. Provisional Application No. 61/322,211, filed Apr. 8, 2010, which is herein incorporated by reference in its entirety. This application is also a continuation-in-part of U.S. application Ser. No. 12/055,936, filed Mar. 26, 2008, which is a continuation of U.S. application Ser. No. 11/632,289, filed Dec. 12, 2007, which was the National Stage of International Application No. PCT/US05/26095, filed Jul. 22, 2005, which claims the benefit of U.S. application Ser. No. 10/710,588, filed Jul. 22, 2004, now abandoned, which applications are herein incorporated by reference in their entireties.

### BACKGROUND OF THE INVENTION

According to several sources, there are 30 to 50 million people in the world who are lactose intolerant. In the 1960's and 1970's, it was reported that 70% of the adults in the world had lactose intolerance. In 1995, it was reported that 75% of the adults in the world and 25% of the adults in the U.S. were categorized as being lactose intolerant. In 1994, it was reported that 75% of African Americans and Native Americans and 90% of Asian Americans had lactose intolerance. It has also been reported that 30% of adults who are mostly North Western and North American descendants of the Europeans, have adapted to high lactase activity into adulthood. Research concludes that this adaptation is genetically controlled, permanent, and related to a long tradition of milk and milk product consumption in these regions of the world.

Lactose intolerance is the inability to digest significant amounts of lactose, a major natural sugar found in milk and milk products of all mammals. Lactose intolerance is caused by a shortage of the enzyme lactase, which is produced by the cells that line the small intestine and is essential to lactose digestion. Lactase breaks down the lactose, a disaccharide, into two simpler forms of sugar called glucose and galactose, which are then transported across the cell membrane and absorbed into the bloodstream. If lactase is not present, or not present in sufficient levels, excess undigested lactose passes through the small intestines into the large intestine where it is fermented by a bacteria in the colon (“colonic flora”, “gut flora”, or “intestinal flora”). The fermentation of the lactose in the large intestine produces hydrogen and methane which can lead to bloating, gas, and diarrhea. These symptoms are caused by a very low activity of lactase in the intestines and are found in individuals who are lactose intolerant. Not all people deficient in lactase have the symptoms commonly associated with lactose intolerance, but those who do are said to have lactose intolerance.

If an individual suspects that he or she has lactose intolerance, it is potentially harmful for him or her to restrict his or her diet since it can result in a nutrition shortage or a failure to detect a more serious disease. Milk and other dairy products are major sources for nutrition in the basic American diet. The primary nutrients in milk are protein, calcium, riboflavin, vitamin A, and vitamin D. Calcium is an important part of the recommended daily allowances of vitamins and minerals and any deficiency therein can lead to health risks such as osteoporosis, hypertension, and/or weak bone density.

Young children who have lactose intolerance are very rare. The amount of lactase enzyme a body produces generally reaches a maximum immediately after birth and then decreases in the majority of people after their body adjusts during the ages of about 3-15.

Generally, humans develop lactose intolerance from a primary or secondary cause. The primary cause is an onset of loss of lactase that is believed to be a permanent condition. This occurs at a variable period after the weaning period. The primary cause is also genetically determined. The secondary cause is generally a temporary condition that occurs as a result of another disease or event that damages the lining of the small intestine where lactase is active. This is usually caused by an acute diarrhea, disease, parasitic infection, Crohn's disease, celiac disease, gastrointestinal surgery, or the intake of certain medications.

In addition to the primary and secondary causes, certain human ethnic and racial populations have more of a predisposition for lactose intolerance. In these populations, social and cultural habits and attitudes influence lactose intolerance. Lactose activity can also decrease with age in certain ethnic and racial populations, including those populations which have origins in Europe, the African plains, and the Siberian Steppes. Humans who are most likely to have or develop lactose intolerance include those of Asian, Middle Eastern, North American, African, and Latin American decent.

### SUMMARY OF THE INVENTION

In one aspect, a composition for increasing lactose tolerance in a subject is provided comprising about 0.1 to 15 grams of lactose formulated for controlled release. In one embodiment, said composition is provided as a capsule or a tablet. In another embodiment, said capsule or tablet comprise an enteric coating. In another embodiment, said composition delivers a substantial amount of lactose to the lower intestine. In another embodiment, said composition does not contain a probiotic. In another embodiment, said composition does not contain a prebiotic. In another embodiment, said composition consists essentially of lactose. In another embodiment, said composition comprises a prebiotic. In another embodiment, said prebiotic comprises FOS. In another embodiment, said composition comprises a probiotic. In another embodiment, said probiotic comprises Lactobacillus or bifidobacteria. In another embodiment, the composition further comprises an enzyme. In another embodiment, said enzyme is lactase.

In another aspect, a composition for increasing lactose tolerance in a subject is provided comprising two or more of lactose, FOS, or Lactobacillus acidophilus, formulated for controlled release. In one embodiment, said composition is provided as a capsule or a tablet. In another embodiment, said capsule or tablet comprise an enteric coating. In another embodiment, said composition comprises about 0.1 to 15 grams of lactose.

In another aspect, a composition for increasing lactose tolerance in a subject is provided comprising lactose, FOS, Lactobaccillus acidiophilus, and a buffer, formulated for controlled release. In one embodiment, said composition is provided as a capsule or a tablet. In another embodiment, capsule or tablet comprise an enteric coating. In another embodiment, said composition comprises 0.1 to 15 grams of lactose.

In another aspect, a method for increasing lactose tolerance in a subject experiencing one or more symptoms of lactose intolerance is provided comprising: administering a composition comprising lactose to the subject for a predetermined number of days, wherein the lactose is formulated for controlled release. In one embodiment, said composition does not contain a probiotic. In another embodiment, said composition does not contain a prebiotic. In another embodiment, said composition consists essentially of lactose. In another embodiment, said composition comprises a prebiotic. In another embodiment, said prebiotic comprises FOS. In another embodiment, said composition comprises a probiotic. In another embodiment, said probiotic comprises Lactobacillus acidophilus. In another embodiment, said composition further comprises an enzyme. In another embodiment, said enzyme is lactase. In another embodiment, said controlled release formulation comprises an enteric coating. In another embodiment, said controlled release formulation releases a substantial amount of lactose in the subject's lower intestine. In another embodiment, a lower amount of lactose is administered on the first day of administration than the last day of administration. In another embodiment, the same amount of lactose is administered on the first day of administration as the last day of administration. In another embodiment, said method comprises administering said composition once a day. In another embodiment, said method comprises administering said composition twice a day. In another embodiment, said method comprises administering said composition three times a day. In another embodiment, said composition comprising lactose is provided as a powder, a tablet or a capsule. In another embodiment, said composition comprising lactose is administered without meals. In another embodiment, said composition comprising lactose is administered in conjunction with meals. In another embodiment, said composition comprising lactose is administered with breakfast and dinner. In another embodiment, said composition comprising lactose is administered with breakfast, lunch and dinner. In another embodiment, said one or more symptoms comprise flatulence, heartburn, stomach upset, nausea, bloating, flatulence, diarrhea, abdominal pain, cramping, or vomiting. In another embodiment, said administration results in reduction of bloating, diarrhea, gastric distention, pain, or flatulence from the consumption of dairy products and other lactose containing compositions. In another embodiment, after said predetermined number of days, said lactose intolerance remains partially, substantially, or completely eliminated or decreased in severity in a subject for at least about 1 day, 1 week, 1 month, 2 months, 3 months, 4 months, 5 months, 6 months, 9 months, one year, 18 months, two years, three years, four years, or five years. In another embodiment, said subject has a psychological aversion to dairy products. In another embodiment, said administering results in a decrease in said psychological aversion to dairy products. In another embodiment, said subject is an elderly person. In another embodiment, said subject has osteoporosis. In another embodiment, the predetermined number of days is about 1 to 60 days. In another embodiment, said subject has a calcium deficiency. In another embodiment, said subject is an elderly adult. In another embodiment, said subject is a postmenopausal woman. In another embodiment, said composition comprises about 0.1 to 12 grams of lactose. In another embodiment, said predetermined number of days is until a symptom of lactose intolerance decreases.

In another aspect, a method for increasing lactose tolerance in a subject is provided comprising administering a non-aqueous composition to the subject consisting essentially of lactose for a predetermined number of days. In one embodiment, said composition is a controlled release composition. In another embodiment, said composition comprises an enteric coating.

In another aspect, a method for increasing lactose tolerance in a subject experiencing one or more symptoms of lactose intolerance is provided comprising: administering a composition comprising lactose, FOS, Lactobaccillus acidiophilus to the subject for a predetermined number of days, wherein the composition is formulated for controlled release. In one embodiment, controlled release formulation comprises an enteric coating. In another embodiment, said controlled release formulation releases a substantial amount of lactose in the subject's lower intestine.

In another aspect, a method for increasing lactose tolerance in a subject experiencing one or more symptoms of lactose intolerance is provided comprising: administering a composition comprising lactose, FOS, Lactobacillus acidiophilus, and a buffer to the subject for a predetermined number of days, wherein the composition is formulated for controlled release. In one embodiment, said controlled release formulation comprises an enteric coating. In another embodiment, said controlled release formulation releases a substantial amount of lactose in the subject's lower intestine.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

Described herein are methods, compositions, kits, and business methods useful for the reduction of symptoms of lactose intolerance in a subject in need thereof, and for improving overall gastrointestinal (GI) health. Symptoms of lactose intolerance include, but are not limited to, gas, heartburn, stomach upset, bloating, flatulence, diarrhea, abdominal pain, cramping, or vomiting. Minor digestive problems related to the GI also include occasional bloating, diarrhea, constipation, gas, heartburn, or stomach upset. The methods and compositions described herein can be useful for reducing or eliminating one or more of these symptoms, for example through colonic adaptation. Fructose and sorbitol malabsorption are also common when lactose malabsorption is present. The methods and compositions described herein can also be useful for reducing or eliminating malabsorption of saccharides or carbohydrates such as lactose, fructose, or sorbitol.

In one aspect of the methods described, the reduction or elimination of symptoms persists after treatment of a condition has concluded. Thus, the described methods need not be used on a continuous basis but rather can be utilized in a discrete time period and then discontinued. In another aspect of the methods, reduction or elimination of symptoms can be temporary and after an amount of time has passed, treatment can be administered when symptoms reappear to maintain the effects of the methods described herein. In yet another aspect of the methods, the methods described can be administered on a regular basis for reducing symptoms of lactose intolerance and for improving overall gastrointestinal (GI) health.

Provided herein are lactose compositions that are useful for treatment of lactose intolerance, reduction of symptoms of lactose intolerance, and for improving overall gastrointestinal (GI) health. Methods of delivering a therapeutic composition comprising lactose are also provided. In one embodiment, a therapeutic composition comprising lactose does not comprise a probiotic component or a prebiotic component. In another embodiment, a therapeutic composition comprising lactose comprises a probiotic component and no prebiotic component. In another embodiment, a therapeutic composition comprising lactose comprises a prebiotic component and no probiotic component. In a further embodiment, a therapeutic composition comprising lactose comprises both a probiotic component and a prebiotic component. In another embodiment, a therapeutic composition consists essentially of lactose. In another embodiment, a therapeutic composition consists of lactose.

### Treatment of Lactose Intolerance

The invention provides methods and compositions useful for the reduction of symptoms of lactose intolerance and for improving overall gastrointestinal (GI) health. Lactose Intolerance, otherwise referred to as lactose maldigestion, is the inability to digest a significant amount of lactose, derived from a deficiency of the lactase enzyme in the small intestine. Lactose is the natural sugar in milk and milk products of all mammals. Lactase is the enzyme which splits the milk sugar lactose into its components (i.e., glucose and galactose), and also breaks down the milk sugar into smaller forms that can be processed into the bloodstream. The lactase enzyme is necessary for mammals to digest lactose. There is an important distinction between lactose intolerance and milk allergies. Lactose intolerance is the inability of the body to digest lactose-containing products due to a deficiency in the lactase enzyme. A milk allergy, however, is a sensitively to the protein in milk, which involves the immune system and does not relate to a deficiency of the lactase enzyme. In humans, a milk allergy is usually experienced only by infants.

Symptoms of lactose intolerance include gas, bloating, diarrhea, abdominal pain, cramping, and vomiting. Minor digestive problems related to the GI also include occasional bloating, diarrhea, constipation, gas, heartburn, or stomach upset. The methods and compositions described herein can be useful for reducing or eliminating one or more of these symptoms, for example through colonic adaptation. These compositions are expected to modify the colonic flora, which can result in an increased tolerance to lactose and other fermentable carbohydrates. Furthermore, these compositions allow the colonic flora, comprising microorganisms known to increase the ability of an individual to tolerate fermentable carbohydrates, to be regularly replenished through consumption of the compositions. Adaptation of the colonic flora allows the colon's capacity to handle gas to be used for other challenges and by improving the composition of the colonic flora, the capacity for compositions without decreased lactose amounts is increased. In one embodiment, an individual's tolerance to dairy in general can be improved through regular consumption of these lactose compositions with decreased lactose content. This change in colonic flora is useful for the reduction of bloating, diarrhea, gastric distention and pain, and/or flatulence from the consumption of dairy products and other lactose compositions. In one embodiment, a method of treating lactose intolerance is disclosed. In another embodiment, a method of treating at least one symptom of lactose intolerance is disclosed. In one embodiment, a method of increasing lactose tolerance is disclosed.

There are three types of lactose intolerance. Primary lactose intolerance results from a decrease in lactase production as one ages. Secondary lactose intolerance results when the small intestine decreases lactase production after an illness, surgery, or injury to the small intestine. Secondary lactose intolerance can occur as a result of Crohn's disease, celiac disease, or gastroenteritis. This type of lactose intolerance can be temporary or permanent. A third type of lactose intolerance is congenital lactose intolerance, in which a person is born with lactose intolerance. Risk factors that can make a person more prone to lactose intolerance include, for example, age (lactose intolerance usually has an onset of after age 5), ethnicity (lactose intolerance is more common in black, Asian, Hispanic and American Indian populations), and premature birth (infants born 28 to 32 weeks of gestation).

### Testing for Lactose Intolerance

Lactose intolerance can be tested either indirectly or directly. There are several ways to test by indirect methods: a hydrogen breath test, a stool acidity test, a blood glucose test, or milk challenge test. In the hydrogen breath test, the breath is measured to determine the amount of hydrogen produced after consuming a measured amount of lactose, typically 15 g. The lactose is administered by drinking a lactose mixture, and the subject exhales into a vacuum-sealed collection tube at three one hour time intervals. A high level of hydrogen in the breath indicates an improper digestion of lactose. In a stool test, the stool is tested to determine the amount of acid. In a blood glucose test, the blood is tested to determine the amount of glucose (sugar) content after administering a predetermined amount of lactose-containing product to the subject. The direct method measures lactase activity in a mucosal biopsy specimen.

The stool acidity test is typically used to test lactose intolerance in infants and young children. The hydrogen breath test is typically not recommended for young children since dehydration can occur due to diarrhea after ingestion of the lactose-containing drink.

Effectiveness of treatment can be measured in a number of ways. Conventional measurements, such as those described, can be used before and after treatment. Alternatively, or in addition, the amount of lactose-containing product that can be administered before the onset of one or more symptoms can be measured or evaluated before and after treatment. Thus, for example, treatment is considered partially effective if, after treatment, on average less hydrogen is produced with a given dose of lactose.

More commonly, a subject can not precisely test the amount of hydrogen or, e.g., use a blood glucose test to measure effectiveness. Instead, a subject can have a sense of how much lactose-containing product they can consume, and the types and degree of symptoms experienced after such consumption. “Partial” elimination of symptoms of lactose intolerance is any noticeable or measurable increase in the amount of lactose that can be consumed before the onset of symptoms. “Substantial” elimination of symptoms of lactose intolerance, as used herein, encompasses an effect where at least about twice the amount of lactose can be consumed after treatment before the onset of symptoms as could have been consumed before treatment. “Complete” or “substantially complete” elimination of symptoms of lactose intolerance, as used herein, indicates that normal amounts of lactose can be consumed after treatment (i.e., the amount of lactose in a typical diet for the area or culture in which the subject normally lives) without symptoms, or with only the rare occurrence of symptoms. Thus, for example, a subject can be able to consume one half cup (4 oz.) of milk with no, or minimal, symptoms of lactose intolerance. However, consumption of 1 or more cups of milk can cause symptoms of lactose intolerance, such as gas or diarrhea occur. After treatment with a composition or dosing regimen disclosed herein, a subject can find that, 1 and one-half cups of milk can be consumed in a single dose without causing any symptoms of lactose intolerance. This indicates that symptoms of lactose intolerance were substantially eliminated. Alternatively, a subject can find that after treatment a normal diet for their geographical or cultural region can be consumed with no, or rare, symptoms of lactose intolerance. In that case, symptoms of lactose intolerance were completely eliminated.

Alternatively, effectiveness can be measured by percent decrease in symptoms of lactose intolerance. In this measurement, the severity of a predetermined symptom, or set of symptoms is measured before and after treatment, e.g., using pre and post Likert scale. Exemplary symptoms include gas, bloating, diarrhea, cramping, abdominal pain, and vomiting. Any one or more than one, of the symptoms can be measured. For example, a subject can be asked to rate one or more symptoms on a scale of increasing severity from 1 to 5. In one embodiment, a set of symptoms is rated, and the ratings are added; for example, gas, bloating, diarrhea, abdominal pain, and cramping can be rated. Percentage decrease in symptoms from before to after treatment can be calculated, and the symptoms of lactose intolerance can be considered eliminated by that percent decrease (e.g., if there is a 50% decrease in symptoms, then symptoms of lactose intolerance is 50% eliminated).

The milk challenge test is another way to diagnose lactose intolerance. In the milk challenge test, a person fasts overnight, and then the person drinks a glass of milk in the morning. After drinking the milk, nothing else is eaten or drunk for three to five hours. Milk will produce symptoms of lactose intolerance within several hours if a person is lactose intolerant.

A direct test for lactose intolerance involves biopsy of the intestinal lining to measure lactase levels in the lining.

### Types of Lactose Intolerance

People can have different degrees of lactose intolerance. Lactose intolerance can also be psychologically induced. There are also many different variations of lactose intolerance depending on the subject. For example, some subjects cannot have cheese, melted cheese, plain milk, or warm dairy containing products like milk in coffee, while others cannot have any dairy products at all. Also, most lactose intolerant people are limited as to the amount of special “lactose free” foods they can eat that have been manufactured by specified companies. Some examples of these “lactose free” foods are: MOCHA MIX® ice cream, TOFUTTI® ice cream and ice cream sandwiches, LACTAID® brand milk, FORMAGG™ cheese, TOFUTTI® “Better than Cream Cheese”, and margarine.

Agents that Enhance Digestion

Agents that aid the digestion of lactose can comprise lactase. Using lactase tablets can help lactose intolerant people digest milk and milk products. Each lactase tablet typically hydrolyzes up to 99% of the ingested lactose within 24 hours, and is designed to be ingested with the lactose containing food. Still other techniques for dealing with lactose maldigestion are to use microgranules containing bioactive compounds or microorganisms (see, e.g., U.S. Pat. No. 5,952,021, which is herein incorporated by reference in its entirety). The use of an active lactase composition for treatment of lactase deficiency is described in U.S. Pat. No. 3,718,739, which is herein incorporated by reference in its entirety. Digestive Advantage™ Lactose Intolerance Therapy, which includes probiotics and digestive enzymes, can be used for dietary management of lactose intolerance.

In one embodiment, one or more symptoms of lactose intolerance in a subject exhibiting symptoms of lactose intolerance are decreased or eliminated by administering to the subject increasing amounts of a therapeutic composition comprising, consisting essentially of, or consisting of lactose and an agent that enhance digestion of lactose (e.g., lactase). In one embodiment, one or more symptoms of lactose intolerance in a subject exhibiting symptoms of lactose intolerance are decreased or eliminated by administering to the subject increasing amounts of a therapeutic composition comprising, consisting essentially of, or consisting of lactose and lactase. In one embodiment, the therapeutic composition comprises, consists essentially of, or consists of lactose and lactase. In another embodiment, the therapeutic composition comprises, consists essentially of, or consists of lactose, lactase, and a prebiotic. In another embodiment, the therapeutic composition comprises, consists essentially of, or consists of lactose, lactase, a prebiotic, and a probiotic.

### Modulating Psychological Aversion to Dairy Products

In one embodiment, a subject with a psychological aversion to dairy products can be administered a therapeutic composition comprising, consisting essentially of, or consisting of lactose to modulate the psychological aversion to dairy products. In another embodiment, a subject with a psychological aversion to dairy products can be administered a therapeutic composition comprising, consisting essentially of, or consisting of lactose and a prebiotic to modulate the psychological aversion to dairy products. In another embodiment, a subject with a psychological aversion to dairy products can be administered a therapeutic composition comprising, consisting essentially of, or consisting of lactose and a probiotic to modulate the psychological aversion to dairy products. In another embodiment, a subject with a psychological aversion to dairy products can be administered a therapeutic composition comprising, consisting essentially of, or consisting of lactose, a probiotic, and a prebiotic to modulate the psychological aversion to dairy products. In one embodiment, the modulation can be a decrease in psychological aversion to dairy products. In another embodiment, the modulation of the psychological aversion can result in an increase in consumption of dairy products by the subject. In another embodiment, modulation of the psychological aversion can result in increased blood calcium levels or bone density in the subject. In one embodiment, the subject is a preterm newborn, a full term newborn, an infant up to one year of age, a young child (e.g., 1 yr to 12 yrs), a teenager, (e.g., 13-19 yrs), an adult (e.g., 20-64 yrs), an elderly adult (65 yrs and older) or a pregnant women. In one embodiment, the subject is an elderly adult. In another embodiment, the subject has osteoporosis. In another embodiment, the subject is an elderly adult who has osteoporosis. In another embodiment, the subject is a woman over the age of about 30, 40, 50, 60, 70, 80, 90, 100, or 110 years old. In another embodiment, the woman is a postmenopausal woman.

### Nutritional Deficiency

A subject that has lactose intolerance can restrict his or her diet, which can result in a nutrition shortage and/or disease. Milk and other dairy products are major sources for nutrition in the basic American diet. The primary nutrients in milk are protein, calcium, riboflavin, vitamin A, and vitamin D. Calcium is an important part of the recommended daily allowances of vitamins and minerals and any deficiency therein can lead to health risks such as osteoporosis, hypertension, and/or weak bone density. Thus, if the subject restricts intake of dairy products, e.g., because of lactose intolerance, the individual can become deficient in calcium which can result in bone loss, osteoporosis, hypertension, and/or weak bone density. In one embodiment, a subject that restricts his or her intake of dairy products because of lactose intolerance can be administered a therapeutic composition comprising, consisting essentially of, or consisting of lactose to modulate the restriction of dairy products. In another embodiment, a subject that restricts his or her intake of dairy products because of lactose intolerance can be administered a therapeutic composition comprising, consisting essentially of, or consisting of lactose and a prebiotic to modulate the restriction of dairy products. In one embodiment, a subject that restricts his or her intake of dairy products because of lactose intolerance can be administered a therapeutic composition comprising, consisting essentially of, or consisting of lactose and a probiotic to modulate the restriction of dairy products. In one embodiment, a subject that restricts his or her intake of dairy products because of lactose intolerance can be administered a therapeutic composition comprising, consisting essentially of, or consisting of lactose and a prebiotic and a probiotic to modulate the restriction of dairy products. In one embodiment, the subject is a preterm newborn, a full term newborn, an infant up to one year of age, a young child (e.g., 1 yr to 12 yrs), a teenager, (e.g., 13-19 yrs), an adult (e.g., 20-64 yrs), an elderly adult (65 yrs and older) or a pregnant women. In one embodiment, the subject is an elderly adult. In another embodiment, the subject has osteoporosis. In another embodiment, the subject is an elderly adult who has osteoporosis. In another embodiment, the subject is a woman over the age of about 30, 40, 50, 60, 70, 80, 90, 100, or 110 years old. In another embodiment, the subject is a woman with osteoporosis over the age of about 30, 40, 50, 60, 70, 80, 90, 100, or 110 years. In another embodiment, the woman is postmenopausal.

If an individual suspects that he or she has lactose intolerance, it is potentially harmful for him or her to restrict his or her diet since it can result in a nutrition shortage or a failure to detect a more serious disease. Milk and other dairy products are major sources for nutrition in the basic American diet. The primary nutrients in milk are protein, calcium, riboflavin, vitamin A, and vitamin D. Calcium is an important part of the recommended daily allowances of vitamins and minerals and any deficiency therein can lead to health risks such as osteoporosis, hypertension, and/or weak bone density.

### Administration of Lactose Compositions

In one embodiment a therapeutic composition comprising lactose is administered to a subject with symptoms of lactose intolerance. In one embodiment a therapeutic composition comprising lactose is administered in increasing doses over time to a subject who is suffering from lactose intolerance, experiencing symptoms of lactose intolerance, or is need of improving overall gastrointestinal (GI) health. In one embodiment a subject experiences a reduction or elimination of one or more symptoms of lactose intolerance or an improvement in overall gastrointestinal health after administration of a therapeutic composition comprising lactose. The therapeutic composition comprising lactose can optionally comprise a probiotic component or a prebiotic component. In one embodiment, a therapeutic composition comprising lactose is administered in approximately equivalent doses over a period of time to a subject with lactose intolerance or symptoms of lactose intolerance, or to a subject in need of improved gastrointestinal health. In one embodiment a therapeutic composition comprising lactose is administered in increasing doses, for a period of time, to a subject with lactose intolerance or symptoms of lactose intolerance, or to a subject in need of improved gastrointestinal health. The therapeutic composition comprising lactose can be in any suitable form for oral administration, including a liquid, tablet, capsule, or powdered form. In one embodiment a subject in need thereof is treated with a therapeutic composition consisting essentially of lactose. In another embodiment a subject in need thereof is treated with a therapeutic composition consisting essentially of lactose and a probiotic. In another embodiment a subject in need thereof is treated with a therapeutic composition consisting essentially of lactose and a prebiotic.

In one embodiment a therapeutic composition comprising lactose is delivered to a targeted area of the subject's gastrointestinal tract in a substantially undigested form. In one embodiment, the targeted area for release of a therapeutic composition comprising lactose is the large intestines. The therapeutic composition comprising lactose can be delivered to the targeted area in a controlled release formulation. A therapeutic composition comprising lactose can also be delivered to the targeted area in a formulation with an enteric coating. The enteric coating prevents release of a therapeutic composition comprising lactose prior to reaching the targeted area.

In one embodiment, other substances can be administered in combination with a therapeutic composition comprising lactose. In one embodiment a probiotic or prebiotic component is simultaneously administered with a therapeutic composition comprising lactose. In one embodiment a probiotic or prebiotic component is administered before a therapeutic composition comprising lactose (e.g., before a regimen of increasing doses of a therapeutic composition comprising lactose begins, or before a dose of a therapeutic composition comprising lactose during such a regimen). In another embodiment the probiotic or prebiotic component is administered after a dose of a therapeutic composition comprising lactose (e.g., after a regimen of increasing doses of a therapeutic composition comprising lactose begins, or after a dose of lactose compositions during such a regimen). In another embodiment, the probiotic or prebiotic component can be administered simultaneously with, before, or after the administration of a therapeutic composition comprising lactose or any combination thereof.

In another embodiment a therapeutic composition comprising lactose is supplemented with one or more prebiotics (indigestible saccharides), such as inulin, fructooligosaccharides (FOS), galactooligosaccharides (GOS), lactulose, raffinose, or stachyose. In another embodiment a therapeutic composition comprising lactose is supplemented with one or more strains of probiotic bacteria. In another embodiment a therapeutic composition comprising lactose is supplemented with one or more digestible saccharides, salts or buffers, e.g., phosphates.

In another embodiment a therapeutic composition comprising lactose is administered in combination with another treatment for lactose intolerance. Other suitable treatments that can be combined with a therapeutic composition comprising lactose include enzymes that digest lactose, such as lactase, or compositions comprising pre-digested lactose.

In one embodiment a method of treatment of lactose intolerance decreases or eliminates the symptoms of lactose intolerance in a subject for an extended period of time after the last administration of a therapeutic composition comprising lactose. In another embodiment the methods described include partially, substantially, or completely decreasing or eliminating the symptoms of lactose intolerance in a subject for a one or more days, weeks, months, or years after the cessation of treatment. In one embodiment one or more symptoms of lactose intolerance are permanently reduced or eliminated in a subject.

Duration of Symptom Relief

In one embodiment, one or more symptoms of lactose intolerance in a subject exhibiting symptoms of lactose intolerance are decreased or eliminated by administering to the subject increasing amounts of a therapeutic composition comprising, consisting essentially of, or consisting of lactose for a period of time. In another embodiment, the therapeutic composition comprises, consists essentially of, or consists of lactose and a probiotic. In another embodiment, the therapeutic composition comprises, consists essentially of, or consists of lactose and a prebiotic. In another embodiment, the therapeutic composition comprises, consists essentially of, or consists of lactose, a prebiotic, and a probiotic. In one embodiment, a symptom of lactose intolerance remains partially, substantially, or completely eliminated or decreased in severity in a subject for at least about 1 day, 1 week, 1 month, 2 months, 3 months, 4 months, 5 months, 6 months, 9 months, one year, 18 months, two years, three years, four years, or five years after the termination of treatment. In another embodiment, a symptom of lactose intolerance remains partially, substantially, or completely eliminated or decreased in severity in a subject for about 1 day to about 30 days, about 1 day to about 365 days, about 1 month to about 12 months, about 1 year to about 5 years, about 1 year to about 10 years, about 10 years to about 20 years, about 20 years to about 30 years, about 30 years to about 40 years, about 40 years to about 50 years, about 50 years to about 60 years, about 60 years to about 70 years, about 70 years to about 80 years, or about 80 years to about 90 years after the termination of treatment. In another embodiment a symptom of lactose intolerance remains partially, substantially, or completely eliminated or decreased in severity in a subject for more than 5 years. In another embodiment a symptom of lactose intolerance is permanently eliminated or decreased in severity in a subject after the termination of treatment. In another embodiment, the methods herein decrease symptoms of lactose intolerance in a subject exhibiting symptoms of lactose intolerance by administering to the subject increasing amounts of a therapeutic composition comprising lactose for a period of time, wherein symptoms of lactose intolerance are substantially eliminated for at least about one month after treatment is terminated.

In another embodiment, a symptom of lactose intolerance in a subject exhibiting symptoms of lactose intolerance is decreased or eliminated by administering to the subject increasing amounts of a therapeutic composition comprising, consisting essentially of, or consisting of lactose for a period of time, wherein the symptoms of lactose intolerance, measured as described herein, are decreased by an average of about 1%, 5%, 10%, 20%, 30%, 40%, 50%, 60%, 70%, 80%, 90%, 95%, or about 100% when compared to symptoms prior to the administration of a therapeutic composition comprising lactose. In another embodiment, a symptom of lactose intolerance in a subject exhibiting symptoms of lactose intolerance is decreased or eliminated by administering to the subject increasing amounts of a therapeutic composition comprising, consisting essentially of, or consisting of lactose for a period of time, wherein the symptoms of lactose intolerance, measured as described herein, are decreased by an average of about 1 to about 100%, about 5% to about 95%, about 10% to about 90%, about 20% to about 80%, about 30% to about 70%, about 40% to about 60%, or about 45% to about 55% when compared to symptoms prior to the administration of a therapeutic composition comprising lactose. In another embodiment, the therapeutic composition comprises, consists essentially of, or consists of lactose and a probiotic. In another embodiment, the therapeutic composition comprises, consists essentially of, or consists of lactose and a prebiotic. In another embodiment, the therapeutic composition comprises, consists essentially of, or consists of lactose, a prebiotic, and a probiotic. An “average” decrease is a decrease as measured in a group of subjects exhibiting symptoms of lactose intolerance, such as about 2, 3, 4, 5, 10, 20, or 30 subjects. In one embodiment, the decrease in or elimination of a symptom of lactose intolerance persists for at least about 1 day, 1 week, 1 month, 2 months, 3 months, 4 months, 5 months, 6 months, 9 months, one year, 18 months, two years, three years, four years, or five years. In one embodiment, the decrease in or elimination of a symptom of lactose intolerance persists for at least about 1 day to about 7 days, about 1 day to about 30 days, about 1 month to about 12 months, about 1 year to about 5 years, about 5 years to about 10 years, about 10 years to about 20 years or more. In another embodiment a symptom of lactose intolerance remains partially, substantially, or completely eliminated or decreased in severity in a subject for more than 5 years after the termination of treatment. In one embodiment, the decrease or elimination of a symptom is permanent. In another embodiment, the invention provides a method of decreasing symptoms of lactose intolerance in a subject exhibiting symptoms of lactose intolerance by administering to the subject increasing amounts of a therapeutic composition comprising lactose for a period of time, wherein one or more symptoms of lactose intolerance, measured as described herein, are decreased by an average of at least about 20% and remain decreased by at least about 20% for at least about one month after treatment is terminated. In another embodiment, the methods herein decrease symptoms of lactose intolerance in a subject exhibiting symptoms of lactose intolerance by administering to the subject increasing amounts of a therapeutic composition comprising lactose for a period of time, wherein one or more symptoms of lactose intolerance, measured as described herein, are decreased by an average of about least about 50% and remain decreased by at least about 50% for at least about one month after treatment is terminated. In another embodiment, the methods herein decrease symptoms of lactose intolerance in a subject exhibiting symptoms of lactose intolerance by administering to the subject increasing amounts of a therapeutic composition comprising, consisting essentially of, or consisting of lactose for a period of time, wherein one or more symptoms of lactose intolerance, measured as described herein, are decreased by an average of about least about 75% and remain decreased by at least about 75% for at least about one month after treatment is terminated. In another embodiment, the therapeutic composition comprises, consists essentially of, or consists of lactose and a probiotic. In another embodiment, the therapeutic composition comprises, consists essentially of, or consists of lactose and a prebiotic. In another embodiment, the therapeutic composition comprises, consists essentially of, or consists of lactose, a prebiotic, and a probiotic.

In one embodiment, a subject can be given first dose of a therapeutic composition comprising, consisting essentially of, or consisting of lactose for a period of time during a treatment regimen and a second dose of a therapeutic composition comprising, consisting essentially of, or consisting of lactose during a second period of time during the treatment regimen. In another embodiment, the first and/or second dose of the therapeutic composition comprises, consists essentially of, or consists of lactose and a probiotic. In another embodiment, the first and/or second dose of the therapeutic composition comprises, consists essentially of, or consists of lactose and a prebiotic. In another embodiment, the first and/or second dose of the therapeutic composition comprises, consists essentially of, or consists of lactose, a prebiotic, and a probiotic. In one embodiment the first and second dose differ in amounts of lactose or, optionally amounts of a prebiotic or a probiotic. For example, a subject can be administered a first dose of a therapeutic composition comprising lactose (e.g., X grams of lactose) for a one or two week period, and a second dose of a therapeutic composition comprising lactose (e.g., 1.1 to 3×X grams of lactose) for a subsequent one or two week period.

Dosage and Administration

In one embodiment the maximum amount of lactose composition per administration is between 0.4 g and 20 g. In another embodiment, the maximum amount of lactose composition per administration is between about 0.1 g to 15 g, about 0.5 g to about 19.5 g, about 0.6 g to about 19 g, about 0.7 g to about 18.5 g, about 0.8 g to about 18 g, about 0.9 g to about 17.5 g, about 1 g to about 17 g, about 1.5 g to about 16.5 g, about 2 g to about 16 g, about 2.5 g to about 15.5 g, about 3 g to about 15 g, about 3.5 g to about 14.5 g, about 4 g to about 14 g, about 4.5 g to about 13.5 g, about 5 g to about 13 g, about 5.5 g to about 12.5 g, about 6 g to about 12 g, about 6.5 g to about 11.5 g, about 7 g to about 11 g, about 8.5 g to about 10.5 g, or about 9 g to about 10 g. In another embodiment the maximum amount of lactose administered to a subject per day is about 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20 g per day. In another embodiment, a dose can be about 0.4 g to 6 g of lactose per day. In another embodiment the maximum amount of lactose administered to a subject per day is about 0.1 g to 15 g, about 0.5 g to about 19.5 g, about 0.6 g to about 19 g, about 0.7 g to about 18.5 g, about 0.8 to about 18 g, about 0.9 g to about 17.5 g, about 1 g to about 17 g, about 1.5 g to about 16.5 g, about 2 g to about 16 g, about 2.5 g to about 15.5 g, about 3 g to about 15 g, about 3.5 g to about 14.5 g, about 4 g to about 14 g, about 4.5 g to about 13.5 g, about 5 g to about 13 g, about 5.5 g to about 12.5 g, about 6 g to about 12 g, about 6.5 g to about 11.5 g, about 7 g to about 11 g, about 8.5 g to about 10.5 g, about 9 g to about 10 g, about 0.4 g to about 1 g, about 1 g to about 2 g, about 2 g to about 5 g, about 5 g to about 10 g, about 10 g to about 15 g, or about 15 g to about 20 g.

In one embodiment increasing dosage of a lactose composition can be achieved by increasing the number of doses per day of the composition administered, increasing the amount of the composition per dose, or both. In one embodiment, both strategies are used. Thus, in one embodiment, a therapeutic composition comprising lactose, is initially administered once per day, at increasing doses, followed by twice per day administration, also at increasing doses. In one embodiment, a dose can be administered to a subject at a frequency of once per day, twice per day, or three times per day. The number of days of administration can last for a period of about 1 to 60 days, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, 41, 42, 43, 44, 45, 46, 47, 48, 49, 50, 51, 52, 53, 54, 55, 56, 57, 58, 59, or 60 days, or about 7-28 days, about 7-14 days, about 7-21 days, about 2-4 weeks, about 1-60 days, about 10-54 days, or about 10-14 days. The length of the program can be about 10 to 14 days to induce tolerance to dairy products, then more than about 20 days to overcome aversion to dairy products.

In another embodiment, a therapeutic composition comprising lactose is administered twice per day. The first dose of a therapeutic composition comprising lactose can remain constant while the second dose increases over time. In another embodiment, a therapeutic composition comprising lactose can be administered an average of about once per day, twice per day, three, four, five, six, or more than six timer per day, or any combination thereof.

In another embodiment a therapeutic composition comprising lactose is administered at the same dosage level at each administration. Thus, in one embodiment, a therapeutic composition comprising lactose is initially administered once to six times per day at the same dosage level. A therapeutic composition comprising lactose can be administered for a period of about 3 to 60 days, such as about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In another embodiment, a therapeutic composition comprising lactose can be administered for a period of about 1 day to about 60 days, about 5 days to about 55 days, about 10 days to about 50 days, about 15 days to about 45 days, about 20 days to about 40 days, about 25 days to about 35 days, about 1 day to about 10 days, about 10 days to about 20 days, about 20 days to about 30 days, about 30 days to about 40 days, about 40 days to about 50 days, or about 50 days to about 60 days.

In one embodiment, a subject who has completed a treatment regimen consumes dairy products at least once every 4-5 days in order to maintain the reduction in symptoms of lactose intolerance.

In another embodiment, a subject self-administers a therapeutic composition comprising lactose. In another embodiment, a therapeutic composition comprising lactose is supplied or recommended by a health professional, e.g., a dietician, nutritionist, nurse, physician, or other qualified health professional. In another embodiment, a therapeutic composition comprising lactose is administered by a health professional or results of the program are monitored by a health professional. In one embodiment, a therapeutic composition comprising lactose is labeled as a medical food.

In one embodiment a starting dose of a therapeutic composition comprising lactose is administered to a subject in need thereof as part of a dosing regime with incremental increases in the dosage of the therapeutic composition comprising lactose over time. The incremental increases in a therapeutic composition comprising lactose dosage can be any suitable dose size. In one embodiment, the starting dose of a therapeutic composition comprising lactose is about 0.1 g to 15 g, about 0.05 g to 4.0 g, or about 0.1 g to about 3 g, or about 0.2 g to about 3.0 g, or about 0.2 g to about 2 g, or about 0.4 g to about 1.6 g, or about 0.4 g to about 1.4 g, or about 0.6 g to about 1.2 g, or about 0.6 g to about 1.0 g, or about 0.7 g to about 0.9 g, or about 0.8 g. In another embodiment, the starting dose of a therapeutic composition comprising lactose is about 0.2 g to about 4.7 g, 0.5 g to about 8.0 g, or about 0.4 g to about 6.8 g. The incremental increase in a therapeutic composition comprising lactose dosage can vary, or each increase can be the same, or any combination thereof. The therapeutic composition comprising lactose can increase incrementally by about 0.1 g to 15 g, about 0.05 g to 4.0 g, or about 0.1 g to about 3 g, or about 0.2 g to about 3.0 g, or about 0.2 g to about 2 g, or about 0.4 g to about 1.6 g, or about 0.4 g to about 1.4 g, or about 0.6 g to about 1.2 g, or about 0.6 g to about 1.0 g, or about 0.7 g to about 0.9 g, or about 0.8 g. The therapeutic composition comprising lactose can increase incrementally by about 0.5 g, about 0.29 g, about 0.30 g, or about 0.42 g, about 0.43 g. The maximum dose reached in treatment again can be any suitable dose size, depending on the subject being treated and the outcome desired. In one embodiment the maximum dose of a therapeutic composition comprising lactose administered in a single dose can be about 0.1 g to 15 g, about 6 g to about 60 g, or about 12 g to about 48 g, or about 14 to about 36 g, or about 16 to about 36 g, or about 18 to about 34 g, or about 20 g to about 32 g, or about 22 g to about 30 g, or about 23 g to about 29 g, or about 24 g to about 28 g, or about 25 to about 27 g, or about 25.5 g to about 26.5 g, or about 25.5 g, 25.6 g, or 25.7 g. In one embodiment the maximum dose of a therapeutic composition comprising lactose administered is about 12 g.

In one embodiment of the invention, the initial dose of a therapeutic composition comprising lactose is about 0.4 g, and the dose is increased by 0.4 g over time, for example, daily, until a maximum dose of 20 g to 25 g of a therapeutic composition comprising lactose is reached. In another embodiment, the initial dose of a lactose composition is about 0.5 g, and the dose is increased by 0.5 g over time, for example, daily, until a maximum dose of 8.0 g to 15 g of lactose composition is reached.

A therapeutic composition comprising lactose can be given in any suitable form, such as a powder, capsules, tablets, a powder that can be dissolved in a liquid prior to consumption, or in liquid form, (e.g., lactose pre-dissolved in a liquid). Any grade or form of lactose that is suitable for consumption by the subject being treated, e.g., by a human, can be used.

Additional Substances

Additional substances can be given in conjunction with a therapeutic composition comprising lactose. These substances can enhance the action of the increasing doses of lactose by, e.g., encouraging the growth of bacteria in the gut that alleviate symptoms of lactose intolerance, increasing adhesion of friendly bacteria, or allowing doses of friendly bacteria to more readily pass through the stomach without being destroyed. These substances can be given prior to treatment with a therapeutic composition comprising lactose, during treatment with a therapeutic composition comprising lactose, after treatment with a therapeutic composition comprising lactose, or any combination thereof. If administered during a therapeutic composition comprising lactose treatment, they can be co-administered with a dose of a therapeutic composition comprising lactose or administered before or after a dose of a therapeutic composition comprising lactose, or any combination thereof.

Substances of use in the invention in conjunction with a therapeutic composition comprising lactose further comprises a probiotic component, a prebiotic component, lactase or other lactose digestive enzymes, and buffers (such as phosphates). The probiotic component comprises one or more strains of live probiotic bacteria. In one embodiment, during some or all of the treatment, a therapeutic composition comprising lactose further comprises probiotic bacteria. In another embodiment, during some or all of the treatment regimen, a therapeutic composition comprising lactose further comprises one or more prebiotics. Prebiotics are indigestible saccharides such as inulin or fructooligosaccharides, or galactooligosaccharides, In one embodiment indigestible saccharides make up about 10% by weight or less (such as about 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%) of a therapeutic composition comprising lactose. In one embodiment, prebiotics make up by weight about 0.1% to about 10%, about 1% to about 10%, about 2% to about 10%, about 3% to about 10%, about 4% to about 10%, about 5% to about 10%, about 4% to about 9%, about 5% to about 8%, about 0.1% to about 1%, about 1% to about 3%, about 3% to about 5%, about 5% to about 7%, about 7% to about 9%, or about 8% to about 10% of a therapeutic composition comprising lactose.

In another embodiment, during some or all of the treatment regimen, a therapeutic composition comprising lactose further comprises lactase or other lactose digestive enzymes. In another embodiment, during some or all of the treatment regimen, a therapeutic composition comprising lactose further comprises a buffer (e.g., phosphates). In another embodiment, during some or all of the treatment, a therapeutic composition comprising lactose further comprises digestible saccharides other than lactose, such as glucose or galactose. In one embodiment the digestible saccharides make up about 10% by weight or less (such as about 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%) of a therapeutic composition comprising lactose. In one embodiment, digestible saccharides make up by weight about 0.1% to about 10%, about 1% to about 10%, about 2% to about 10%, about 3% to about 10%, about 4% to about 10%, about 5% to about 10%, about 4% to about 9%, about 5% to about 8%, about 0.1% to about 1%, about 1% to about 3%, about 3% to about 5%, about 5% to about 7%, about 7% to about 9%, or about 8% to about 10% of a therapeutic composition comprising lactose.

In one embodiment, a therapeutic composition comprises, consists essentially of, or consists of lactose and lactase. In another embodiment, a therapeutic composition comprises, consists essentially of, or consists of lactose, lactase, and a probiotic. In another embodiment, a therapeutic composition comprises, consists essentially of, or consists of lactose, lactase, and a prebiotic. In another embodiment, a therapeutic composition comprises, consists essentially of, or consists of lactose, lactase, a prebiotic, and a probiotic.

Duration of Treatment with Lactose Compositions

The total duration of treatment can be from about one week to about 12 weeks, or about four weeks to about ten weeks, or about four weeks to about eight weeks, or about six weeks. During this period of time, the subject is started on a program of taking increasing amounts of a therapeutic composition comprising lactose, optionally along with ingestion of lactose containing food products. In one embodiment a lactose composition further comprises a probiotic or a prebiotic, as described herein. In one embodiment, the total duration of treatment is about 5 days to about 40 days. In one embodiment, the total duration of treatment is about 7 days to about 90 days, or about 7 days to about 60 days, or about 14 days to about 50 days, or about 14 days to about 40 days, or about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days. In another embodiment, the total duration of treatment is about 30 days. In another embodiment, the total duration of treatment is about 34 days. In another embodiment, the total duration of treatment is about 36 days. In another embodiment, the total duration of treatment is about 38 days. In another embodiment, the total duration of treatment is about 42 days. In another embodiment, the total duration of treatment is about 60 days. In another embodiment, the total duration of treatment is about 90 days.

In another embodiment, the total duration of treatment is based on a subject's response to the treatment. For example, an individual can experience a reduction in lactose intolerance symptoms after 14 days of treatment with a therapeutic composition comprising lactose. In another example an individual can experience a reduction in lactose intolerance symptoms after 30 days of treatment with a therapeutic composition comprising lactose. Thus, the duration of treatment is determined by an individual subject's response to a therapeutic composition comprising lactose and the onset of relief from one or more lactose intolerance symptoms.

In one embodiment the treatment is continuous and on-going. In one embodiment, the duration of the treatment is based on a subject's symptoms of lactose intolerance. Thus, a subject can experience partial relief of one or more symptoms of lactose intolerance at a first dosage amount of a therapeutic composition comprising lactose, and the subject can continue administration at the first dosage amount, or a lower dosage amount, until complete relief of the one or more symptoms of lactose intolerance is achieved. Thus, in one embodiment, the duration of the treatment is not definitively established at the outset, but continues until the desired level of lactose tolerance or symptom relief is achieved.

In one embodiment of the invention a subject in need thereof can have repeated courses of treatment with a therapeutic composition comprising lactose. The course of treatment can be repeated when symptoms of lactose intolerance reappear or increase to an undesirable level. Alternatively, the course of treatment can be repeated at regular or predetermined intervals. Thus, treatment can be repeated after about one month, two months, three months, four months, six months, eight months, ten months, one year, 18 months, two years, three years, four years, five years, or more than five years, or any combination thereof (e.g., treatment can be repeated after one year, then every two to five years thereafter). The treatment can be repeated in the same form (e.g., duration, dosage, timing of dosage, additional substances, etc.) as used in the first treatment or it can be modified. For example, treatment duration can be shortened or lengthened, dosage can be increased more quickly or slowly or a higher or lower starting dose of a prebiotic composition, a different therapeutic composition comprising lactose (such as a composition comprising a probiotic component, a prebiotic component, or both) can be used (e.g., containing more or less of other substances, or fewer or more substances in addition to lactose), and the like.

### Methods of Delivery

Methods of the invention include methods of administering lactose in increasing doses to an individual suffering from lactose intolerance. The end result is a reduction or elimination of the symptoms of lactose intolerance in the individual. In one embodiment a therapeutic composition comprising lactose is administered to a subject with lactose intolerance in increasing doses, for a pre-determined period of time. In another embodiment a therapeutic composition comprising lactose is administered to a subject with lactose intolerance at a constant amount, for a pre-determined period of time. In another embodiment, a therapeutic composition comprising lactose is administered to a subject with lactose intolerance for a first period of time in increasing doses followed by a second period of time wherein a therapeutic composition comprising lactose is administered in constant doses. A therapeutic composition comprising lactose can be in any form suitable for oral consumption, including liquid, powder, solid or in a compacted form. In one embodiment, other substances are administered in combination with the lactose. “In combination,” as used herein, encompasses simultaneous administration of a substance with lactose, as well as administration before lactose (e.g., before a regimen of increasing doses of lactose begins, or before a dose of lactose during such a regimen), after lactose (e.g., after a regimen of increasing doses of lactose begins, or after a dose of lactose during such a regimen), or any combination thereof. Other substances of use in the methods and compositions of the invention besides lactose include probiotics (e.g., live bacteria), prebiotics (e.g., fructooligosaccharides or galactooligosaccharides) or a buffer (e.g., phosphates). In one embodiment, lactose is administered without a probiotic component (i.e. live bacteria). In another embodiment, the lactose is administered without a prebiotic component (i.e. frutooligosaccharides). In another embodiment a subject with lactose intolerance is administered a therapeutic composition comprising lactose. In another embodiment a subject with lactose intolerance is administered a therapeutic composition comprising lactose that is formulated for controlled release. In one embodiment the controlled release is mediated by an enteric coating. In another embodiment a subject with lactose intolerance is administered a therapeutic composition consisting essentially of lactose. In another embodiment a subject with lactose intolerance is administered a therapeutic composition consisting essentially of lactose that is formulated for controlled release. In one embodiment the controlled release is mediated by an enteric coating.

Methods of the invention also include the administration of lactose in increasing doses, in combination with other treatments for lactose intolerance. Other treatments include any of those described herein, such as the use of lactase, or the use of products containing pre-digested lactose.

Further provided herein are methods of decreasing the symptoms of lactose intolerance for an extended period of time after treatment stops. Thus, the methods of the invention include partially, substantially, or completely decreasing the symptoms of lactose intolerance for a period of days, weeks, months, years, or permanently. Such a decrease is accomplished by the methods and compositions described herein.

Individuals who can benefit from the methods and compositions described herein include individuals suffering from the symptoms of lactose intolerance, as described above. Any degree of lactose intolerance can be treated by the methods of the invention. Symptoms of lactose intolerance include gas, abdominal bloating, abdominal distention, abdominal discomfort, diarrhea, vomiting, and/or cramping.

In one embodiment, the invention provides a method of decreasing symptoms of lactose intolerance in an individual exhibiting symptoms of lactose intolerance by administering to the individual increasing amounts of lactose for a period of time, wherein one or more symptoms of lactose intolerance are partially, substantially, or completely eliminated. In one embodiment, the lactose is administered in constant dosage for a period of time. In an embodiment, the symptom(s) of lactose intolerance remains partially, substantially, or completely eliminated for at least about 1 day, 1 week, 1 month, 2 months, 3 months, 4 months, 5 months, 6 months, 9 months, one year, 18 months, two years, three years, four years, five years, or more than five years after the termination of treatment, or permanently after the termination of treatment. In another embodiment, a symptom of lactose intolerance remains partially, substantially, or completely eliminated or decreased in severity in a subject for about 1 day to about 30 days, about 1 day to about 365 days, about 1 month to about 12 months, about 1 year to about 5 years, about 1 year to about 10 years, about 10 years to about 20 years, about 20 years to about 30 years, about 30 years to about 40 years, about 40 years to about 50 years, about 50 years to about 60 years, about 60 years to about 70 years, about 70 years to about 80 years, or about 80 years to about 90 years after the termination of treatment. In one embodiment, the invention provides a method of decreasing symptoms of lactose intolerance in an individual exhibiting symptoms of lactose intolerance by administering to the individual increasing amounts of lactose for a period of time, wherein symptoms of lactose intolerance are substantially eliminated for at least about one month after treatment is terminated. In one aspect of the methods described, the reduction or elimination of symptoms persists after treatment of a condition has concluded. Thus, the described methods need not be used on a continuous basis but rather can be utilized in a discrete time period and then discontinued. In another aspect of the methods, reduction or elimination of symptoms can be temporary and after an amount of time has passed, treatment can be administered when symptoms reappear to maintain the effects of the methods described herein. In yet another aspect of the methods, the methods described can be administered on a regular basis for reducing symptoms of lactose intolerance and for improving overall gastrointestinal (GI) health.

In one embodiment, the invention provides a method of decreasing symptoms of lactose intolerance in an individual exhibiting symptoms of lactose intolerance by administering to the individual increasing amounts of lactose for a period of time, wherein the symptoms of lactose intolerance, measured as described herein, are decreased by an average of about 1%, 5%, 10%, 20%, 30%, 40%, 50%, 60%, 70%, 80%, 90%, 95%, or about 100%. In one embodiment, the invention provides a method of decreasing symptoms of lactose intolerance in an individual exhibiting symptoms of lactose intolerance by administering to the individual increasing amounts of lactose for a period of time, wherein the symptoms of lactose intolerance, measured as described herein, are decreased by an average of about 1% to about 100%, about 5% to about 95%, about 10% to about 90%, about 20% to about 80%, about 30% to about 70%, about 40% to about 60%, or about 45% to about 55%. An “average” decrease is a decrease as measured in a group of individuals exhibiting symptoms of lactose intolerance, such as about 2, 3, 4, 5, 10, 20, or 30 individuals. In one embodiment, the decrease of symptoms of lactose intolerance persists or becomes even greater (e.g., 50% decrease to 55% decrease) for at least about 1 day, 1 week, 1 month, 2 months, 3 months, 4 months, 5 months, 6 months, 9 months, one year, 18 months, two years, three years, four years, five years, or more than five years after the termination of treatment. In one embodiment, the decrease in symptoms is permanent. In one embodiment, the invention provides a method of decreasing symptoms of lactose intolerance in an individual exhibiting symptoms of lactose intolerance by administering to the individual increasing amounts of lactose for a period of time, wherein the symptoms of lactose intolerance, measured as described herein, are decreased by an average of about least about 20% and remain decreased by at least about 20% for at least about one month after treatment is terminated. In one embodiment, the invention provides a method of decreasing symptoms of lactose intolerance in an individual exhibiting symptoms of lactose intolerance by administering to the individual increasing amounts of lactose for a period of time, wherein the symptoms of lactose intolerance, measured as described herein, are decreased by an average of about least about 50% and remain decreased by at least about 50% for at least about one month after treatment is terminated.

The total duration of treatment can be from about two weeks to about 12 weeks, or about four weeks to about ten weeks, or about four weeks to about eight weeks, or about six weeks. During this period of time, the subject is started on a program of taking increasing amounts of a therapeutic composition comprising lactose of the invention, optionally along with ingestion of lactose containing food products, and in one embodiment also in combination with other substances, as described herein. In one embodiment, the total duration of treatment is about 15 days to about 90 days, or about 15 days to about 60 days, or about 20 days to about 50 days, or about 20 days to about 40 days, or about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days. In one embodiment, the total duration of treatment is about 38 days. In one embodiment, the total duration of treatment is about 42 days. It will be appreciated that these durations are averages, and that individuals using the treatment can vary from the average based on the severity of their symptoms, missing days of treatment, and the like. In one embodiment, the duration of the treatment is based on the individual's symptoms. Thus, an individual can experience a return of symptoms at a given dose of lactose, and can require that they stay at that dose, or a lower dose, until symptoms subside. Thus, in one embodiment, the duration of the treatment is not definitively established at the outset, but continues until the highest dose of lactose is achieved, or until the desired level of lactose tolerance is achieved

Increasing dosage of lactose can be achieved by increasing the number of doses per day of lactose administered, increasing the amount of lactose per dose, or both. Typically, both strategies are used. Thus, in one embodiment of the invention, lactose is initially administered once per day, at increasing doses, followed by twice per day administration, also at increasing doses. The once per day administration can last for a period of about 6 to 30, or about 10 to 26, or about 14 to 24, or about 16 to 20, or about 18 days, and the twice per day administration can last for a period of about 6 to 30, or about 14 to 24, or about 16 to 20, or about 14 to 28, or about 16 days. In one embodiment, during the twice per day administration, the first dose of lactose is constant while the second dose increases. In one embodiment, lactose can be administered an average of about once per day, twice per day, three, four, five, six, or more than six timer per day, or any combination thereof.

After treatment has concluded, the individual is encouraged to enjoy dairy products at least once every 4-5 days in order to maintain the reduction in symptoms of lactose intolerance.

In one embodiment, a subject self-administers a therapeutic composition comprising lactose. In one embodiment, a therapeutic composition comprising lactose is supplied or recommended by a health professional, e.g., a dietician, nutritionist, nurse, physician, or other qualified health professional. In one embodiment, a therapeutic composition comprising lactose is administered by a health professional and/or results of the program are monitored by a health professional. In one embodiment, a therapeutic composition comprising lactose is labeled as a medical food.

While a subject typically will not require more than one course of treatment, in one embodiment of the invention an individual can have repeated courses of treatment. The course of treatment can be repeated when symptoms of lactose intolerance appear or increase to an undesirable level. Alternatively, the course of treatment can be repeated at regular or predetermined intervals. Thus, treatment can be repeated after about one month, two months, three months, four months, six months, eight months, ten months, one year, 18 months, two years, three years, four years, five years, or more than five years, or any combination thereof (e.g., treatment can be repeated after one year, then every two to five years thereafter). The treatment can be repeated in the same form (e.g., duration, dosage, timing of dosage, additional substances, etc.) as used in the first treatment, or it can be modified. For example, treatment duration can be shortened or lengthened, dosage can be increased more quickly or slowly and/or a higher or lower starting dose of lactose can be used, a different therapeutic composition comprising lactose can be used (e.g., containing more or less of other substances, or fewer or more substances in addition to lactose), and the like.

The starting dose of lactose and the incremental increases in lactose dosage can be any suitable dose size. In one embodiment, the starting dose of lactose is about 0.1 g to 15 g, about 0.05 to 4.0 gm, or about 0.1 to about 3 gm, or about 0.2 to about 3.0 gm, or about 0.2 to about 2 gm, or about 0.4 to about 1.6 gm, or about 0.4 to about 1.4 gm, or about 0.6 to about 1.2 gm, or about 0.6 to about 1.0 gm, or about 0.7 to about 0.9 gm, or about 0.8 gm. The incremental increase in lactose dosage can vary, or each increase can be the same, or any combination thereof. The lactose dosage can increase incrementally by about 0.05 to 4.0 gm, or about 0.1 to about 3 gm, or about 0.2 to about 3.0 gm, or about 0.2 to about 2 gm, or about 0.4 to about 1.6 gm, or about 0.4 to about 1.4 gm, or about 0.6 to about 1.2 gm, or about 0.6 to about 1.0 gm, or about 0.7 to about 0.9 gm, or about 0.8 gm. The maximum dose reached in treatment again can be any suitable dose size, depending on the individual being treated and the outcome desired. The maximum dose of lactose can be about 6 to about 60 gm, or about 0.1 to about 12 gm, or about 12 to about 48 gm, or about 14 to about 36 gm, or about 16 to about 36 gm, or about 18 to about 34 gm, or about 20 to about 32 gm, or about 22 to about 30 gm, or about 23 to about 29 gm, or about 24 to about 28 gm, or about 25 to about 27 gm, or about 25.5 to about 26.5 gm, or about 25.5, 25.6, 25.7 gm.

Thus, in one embodiment of the invention, the initial dose of lactose is about 0.8 gm, and the dose is increased by 0.8 gm over time, for example, daily, until a maximum dose of 25.6 gm of lactose is reached. Additional phases of the regimen can include giving various amounts of milk products in which the dosage of lactose can be given in dairy form, before the treatment ends, and the dosage of lactose in the milk products can not be precisely the same as the doses given up to that point; it will be understood that various milk products and brands of milk products can contain varying doses of lactose.

The lactose can be given in any suitable form, i.e., as a powder, such as in capsules or tablets, or powder that can be dissolved in a liquid prior to consumption, or in liquid form, e.g., predissolved in a liquid or in the form of milk. Any grade or form of lactose that is suitable for consumption by the individual being treated, e.g., by humans, can be used.

Optional Substances Administered with Lactose

Additional substances can be given in conjunction with a therapeutic composition comprising lactose. These substances can enhance the action of the increasing doses of lactose by, e.g., encouraging the growth of bacteria in the gut that alleviate symptoms of lactose intolerance, increasing adhesion of friendly bacteria, or allowing doses of friendly bacteria to more readily pass through the stomach without being destroyed. These substances can be given prior to treatment with lactose, during treatment with lactose, after treatment with lactose, or any combination thereof. If administered during lactose treatment, they can be administered with the dose of lactose being given, or before or after the dose of lactose, or any combination thereof.

Substances of use in the invention in conjunction with lactose include a probiotic component (e.g., live bacteria), a prebiotic component (e.g., fructooligosaccharides (FOS)), and buffer components, e.g., phosphates. One or more of these substances can be used in combination with lactose at any suitable time before, during, after treatment, or some combination thereof. Thus, in one embodiment, during some or all of the treatment, lactose is administered in conjunction with a probiotic component (e.g., live bacteria). In one embodiment, during some or all of the treatment, lactose is administered in conjunction with a prebiotic component (e.g., FOS). In one embodiment, during some or all of the treatment, lactose is administered in conjunction with a buffer component (e.g., phosphates). In one embodiment, during some or all of the treatment, lactose is administered in conjunction with a probiotic component (e.g., live bacteria) and a prebiotic component (e.g., FOS). In one embodiment, during some or all of the treatment, lactose is administered in conjunction with a probiotic component and phosphates. In one embodiment, during some or all of the treatment, lactose is administered in conjunction with a prebiotic component and buffer, e.g., phosphates. In one embodiment, during some or all of the treatment, lactose is administered in conjunction with a probiotic component, prebiotic component and buffer components.

### Probiotic Component

Probiotics (or probiotic bacteria) typically refer to beneficial live microorganisms, e.g., live bacteria, found in the gastrointestinal tract and, when administered in adequate amounts, confer a health benefit on the host (or subject in need thereof) such as helping to maintain a healthy immune system, or increasing the ability of the colon to slow fermentation rate. Probiotics favorably alter the intestinal flora balance, inhibit the growth of harmful bacteria, promote good digestion, boost immune function, and increase resistance to infection. People with flourishing intestinal colonies of beneficial bacteria are better equipped to fight the growth of disease-causing bacteria. Any suitable bacteria for assisting in reduction or elimination of lactose intolerance-like symptoms or improving overall GI health, for example through colonic adaptation, can be used in the methods and compositions described herein.

### Probiotic Bacteria

Examples of probiotics include, but are not limited to, those that acidify the colon such as those from the genera Lactobacillus or Bifidobacteria, which are thought to maintain a healthy balance of intestinal flora by producing organic compounds, such as lactic acid, hydrogen peroxide, and acetic acid, resulting in increased acidity of the intestine and inhibiting the reproduction of many harmful bacteria. Probiotics also produce substances called bacteriocins, which act as natural antibiotics to help eliminate undesirable microorganisms. The bacteria can be given as part of a food, e.g., in yogurt, or in powdered form.

Non-exclusive examples of probiotic bacteria that can be used in the methods and compositions described herein include Lactobacillus acidophilus or L. acidophilus. Acidophilus, a probiotic, is an important strain of the Lactobacilli family of gut flora which inhabit the GI tract. These beneficial bacteria are involved with immune system function, inhibiting carcinogenesis, metabolism of cholesterol, aging, and nutritional status. Acidophilus and other probiotics help maintain optimum pH, reduce putrefaction, and reduce endotoxemia. Other Lactobacillus bacteria which can be employed include, but are not limited to, L. crispatus, L. casei, L. rhamnosus, L. reuteri, L. fermentum, L. plantarum, L. sporogenes, and L. bulgaricus. Other probiotic bacteria suitable for the compositions include Bifidobacterium lactis, B. animalis, B. bifidum, and B. infantis. Yeasts, such as Saccharomyces boulardii, are also suitable as probiotics and act to restore the intestinal flora. Mixtures of one or more species or strains of bacteria can be used. For example, yogurt is a product which already contains the bacteria species Lactobacillus bulgaricus and Streptococcus thermophilus used for fermentation and can contain additional species of probiotics and can also be supplemented with prebiotics.

Other strains of probiotic bacteria that can be used in the methods and compositions described herein include, for example, Bacillus coagulans GBI-30, 6086; Bifidobacterium animalis subsp. lactis BB-12; Bifidobacterium breve Yakult; Bifidobacterium infantis 35624; Bifidobacterium animalis subsp. lactis HN019 (DR10); Bifidobacterium longum BB536; Escherichia coli M-17; Escherichia coli Nissle 1917; Lactobacillus acidophilus DDS-1; Lactobacillus acidophilus LA-5; Lactobacillus acidophilus NCFM; Lactobacillus casei DN114-001 (Lactobacillus casei Immunitas(s)/Defensis); Lactobacillus casei CRL431; Lactobacillus casei F19; Lactobacillus casei; Lactobacillus paracasei St11 (or NCC2461); Lactobacillus johnsonii La1 (=Lactobacillus LC1, Lactobacillus johnsonii NCC533); Lactococcus lactis L1A; Lactobacillus plantarum 299V; Lactobacillus reuteri ATTC 55730 (Lactobacillus reuteri SD2112); Lactobacillus rhamnosus ATCC 53013; Lactobacillus rhamnosus LB21; Saccharomyces cerevisiae (boulardii) lyo; mixture of Lactobacillus rhamnosus GR-1 & Lactobacillus reuteri RC-14; mixture of Lactobacillus acidophilus NCFM and Bifidobacterium bifidum BB-12; mixture of Lactobacillus acidophilus CL1285 and Lactobacillus casei; mixture of Lactobacillus helveticus R0052 and Lactobacillus rhamnosus R0011.

In one embodiment, a lactose composition comprises lactose and probiotic. In one embodiment a therapeutic composition comprising lactose comprises or consists essentially of lactose. In one embodiment, a therapeutic composition comprising lactose is administered with increasing doses of probiotics during the period of treatment. In another embodiment, a therapeutic composition comprising lactose is administered with constant doses (dose amounts that do not change) of probiotics during the period of treatment. In another embodiment, a therapeutic composition comprising lactose is administered with both increasing doses of probiotics for a portion of the treatment and a constant dose of probiotics during another portion of the treatment period.

In one embodiment, a probiotic is genetically modified. For example, plasmids encoding specific genes can be introduced into a probiotic. A genetically modified probiotic can be used in the methods and compositions of the provided invention.

### Dose Timing and Size of Probiotics

In an embodiment, a probiotic component is used in or with a therapeutic composition comprising lactose. The probiotic component comprises one or more probiotics. In one embodiment, probiotic bacteria, such as L. acidophilus, are given prior to beginning treatment with lactose. In one embodiment, probiotic bacteria, such as L. acidophilus, is given in conjunction with treatment with lactose, for part or all of the treatment with lactose. Thus, in an embodiment, some or all doses of lactose are accompanied by a dose of bacteria, e.g. live cultured bacteria, e.g., L. acidophilus. In an embodiment, bacteria, e.g., L. acidophilus is given initially with the lactose, but then its use is discontinued. For example, the initial one, two, three, four, five, six, seven, eight, nine, ten, or more than ten days of treatment with lactose can include doses of bacteria, with the use of bacteria discontinued after that time. In one embodiment, bacteria, e.g., bacteria in yogurt, or bacteria by themselves, can be given for the first two days of treatment, then the administration of bacteria is discontinued. In another embodiment, probiotic bacteria, either alone or in combination with other substances or treatments are used after the treatment with lactose is terminated. The bacteria can be administered for any suitable period after the termination of treatment with lactose, and can be administered daily or at regular or irregular intervals. Doses can be as described below.

Any suitable amount of probiotic per serving can be used that allows an effective flora in the GI. Typically, probiotics are given as live cultured bacteria. The dose can be about 0.001 mg to about 1 mg, or about 0.5 mg to about 5 mg, or about 1 mg to about 1000 mg, or about 2 mg to about 200 mg, or about 2 mg to about 100 mg, or about 2 mg to about 50 mg, or about 4 mg to about 25 mg, or about 5 mg to about 20 mg, or about 10 mg to about 15 mg, or about 50 mg to about 200 mg, or about 200 mg to about 1000 mg or about 10, 11, 12, 12.5, 13, 14, or 15 mg per serving. In one embodiment, L. acidophilus is used in a dose of about 12.5 mg. The probiotic bacteria can also be about 0.5 5 w/w to about 20% w/w of the final composition. The dose can be given in combination with lactose. Another common way of specifying the amount of probiotics is as a colony forming unit (cfu). A cfu is an individual cell which is able to clone itself into an entire colony of identical cells. In one embodiment, one or more strains of probiotic bacteria are ingested in an amount of about 1×10⁶ to about 1×10⁹ cfu's, or about 1×10⁶ cfu's to about 1×10⁹ cfu's, or about 10×10⁶ cfu's to about 0.5×10⁹ cfu's, or about 113×10⁵ cfu's to about 113×10⁶ cfu's, or about 240×10⁵ cfu's to about 240×10⁶ cfu's or about 0.3×10⁹ cfu's per serving. In another embodiment, one or more strains of probiotic bacteria are administered as part of a dairy product. In one embodiment, a typical serving size for a dairy product such as fluid milk is about 240 g. In another embodiment, a serving size is about 245 g, or about 240 g to about 245 g, or about 227 to about 300 g. In one embodiment the dairy product is yogurt. Yogurt can have a serving size of about 4 oz, or about 6 oz, or about 8 oz, or about 4 oz to 10 oz, or about half cup, or about 1 cup, or about 113 g, or about 170 g, or about 227 g, or about 245 g or about 277 g, or about 100 g to about 350 g.

In one embodiment probiotic bacteria are given as live cultured bacteria, e.g., in combination with lactose and, optionally, other substances. The dose can be about 1 mg to about 1000 mg, or about 2 mg to about 200 mg, or about 2 mg to about 100 mg, or about 2 mg to about 50 mg, or about 4 mg to about 25 mg, or about 5 mg to about 20 mg, or about 10 mg to about 15 mg, or about 10, 11, 12, 12.5, 13, 14, or 15 mg of probiotic bacteria. In one embodiment, L. acidophilus is used in a dose of about 12.5 mg. In one embodiment, as the administration of lactose dose to a subject increases, the dose of bacteria increases as well. For example, an initial dose of lactose can be about 0.6 g to 1.0 g, e.g., 0.8 g, given in combination with about 10-15 mg, e.g., about 12.5 mg, of L. acidophilus. The dose of lactose can be increased incrementally by about 0.6 g to 1.0 g, e.g., 0.8 g, and the accompanying dose of L. acidophilus can be increased by about 10-15 mg, e.g., about 12.5 mg, of L. acidophilus.

### Prebiotic Component

A prebiotic is a generally a saccharide that is indigestible or essentially indigestible by a human and acts to encourage the growth of probiotic bacteria in the gut that alleviate symptoms of lactose intolerance, increase adhesion of probiotic bacteria in the gut, or allow doses of probiotic bacteria to more readily pass through the stomach without being destroyed. A prebiotic can contain a saccharide that is indigestible and can act as a non-digestible fiber in the diet. This is because humans lack the enzymes to break down some or all of the prebiotic saccharide as it travels through the digestive tract. When the prebiotic reaches the large intestine and the colon, bacteria encoding an enzyme or enzymes capable of digesting the prebiotic break down the prebiotic into simple sugars that the bacteria can use. For example, bifidobacteria have been reported to digest prebiotic saccharides.

In an embodiment, a prebiotic component is used in or with a therapeutic composition comprising lactose. The prebiotic component comprises one or more prebiotics. In yet another embodiment, a therapeutic composition comprising lactose does not comprise a probiotic component but the probiotic component is administered before or after a therapeutic composition comprising lactose is administered. Prebiotics suitable for a prebiotic component can include one or more of a carbohydrate, carbohydrate monomer, carbohydrate oligomer, or carbohydrate polymer. In one embodiment, the prebiotics are indigestible saccharides, which include indigestible monosaccharides, indigestible oligosaccharides, or indigestible polysaccharides. In one embodiment, the sugar units of an oligosaccharide or polysaccharide can be linked in a single straight chain or can be a chain with one or more side branches. The length of the oligosaccharide or polysaccharide can vary from source to source. In one embodiment, small amounts of glucose can also be contained in the chain. In another embodiment, the prebiotics can be partially hydrolyzed.

In one embodiment, a therapeutic composition comprising lactose does not comprise a prebiotic component. In another embodiment, a therapeutic composition comprising lactose comprises a prebiotic component. In yet another embodiment, a therapeutic composition comprising lactose does not comprise a prebiotic component but the prebiotic component is administered before or after a therapeutic composition comprising lactose is administered. In one embodiment, a prebiotic component described herein consists essentially of one or more indigestible saccharides. In another embodiment, the prebiotic component consists essentially of one or more indigestible oligosaccharides.

Effects of Adaptation

In one embodiment a therapeutic composition comprising, consisting essentially of, or consisting of lactose allows the colonic flora, comprising microorganisms known to increase the ability of a subject to tolerate fermentable carbohydrates, to be regularly replenished through consumption of the composition. In another embodiment, the therapeutic composition comprises, consists essentially of, or consists of lactose and a probiotic. In another embodiment, the therapeutic composition comprises, consists essentially of, or consists of lactose and a prebiotic. In another embodiment, the therapeutic composition comprises, consists essentially of, or consists of lactose, a prebiotic, and a probiotic. Adaptation of the colonic flora allows the colon's capacity to handle gas to be used for other challenges; by changing the composition of the colonic flora, the capacity for consumption of compositions with lactose is increased. This change in colonic flora can be useful for the reduction of bloating, diarrhea, gastric distention and pain, or flatulence from the consumption of dairy products and other lactose or containing compositions. In one embodiment, tolerance of a subject to dairy in general can be improved through regular consumption of a therapeutic composition comprising lactose with or without a prebiotic component.

Prebiotics can promote colonic bacteria that slow fermentation. For example, FOS, neosugar, or inulin promote the growth of acid forming bacteria in the colon such as bacteria belonging to the genera Lactobacillus or Bifidobacterium. For instance, L. acidophilus and B. bifidus play a role in reducing the number of pathogenic bacteria. Additional nutritional properties, such as the effect on colonic pH and stool bulking provide for classification as dietary fibers. In experimental models, it has also been reported that they improve the bioavailability of essential minerals. As a fiber, it is thought to slow digestion and allow the painless reintroduction of lactose into the body. Other polymers, such as various galactans, and carbohydrate based gums, such as psyllium, guar, carrageen, gellan, konjac are also known to improve GI health. The carbohydrate lactulose is also known to improve GI gas handling capacity.

In one embodiment, the methods and composition of the provided invention can reduce one or more symptoms of lactose intolerance (e.g., gas, heartburn, stomach upset, bloating, flatulence, diarrhea, abdominal pain, cramping, or vomiting) in a subject by at least 10%, 20%, 30%, 40%, 50%, 60%, 70%, 80%, 90%, or 100%. In another embodiment, the methods of the provided invention can increase lactose digestion in a subject by at least 2-fold, 5-fold, 10-fold, or 100-fold. In another embodiment, the methods and compositions of the provided invention can increase blood calcium levels in a subject by at least 10%, 25%, 50%, 100%, 500%, or 1000%. In another embodiment, the methods and compositions of the provided invention can increase bone density in a subject by at least 10%, 25%, 50%, 100%, 500%, or 1000%. In another embodiment, the methods and compositions of the provided invention can increase tolerance of dairy consumption to 8-14 oz per day.

In one embodiment the prebiotic component comprises one or more of GOS, lactulose, raffinose, stachyose, lactosucrose, FOS (i.e. oligofructose or oligofructan), inulin, isomalto-oligosaccharide, xylo-oligosaccharide, paratinose oligosaccharide, transgalactosylated oligosaccharides (i.e. transgalacto-oligosaccharides), transgalactosylate disaccharides, soybean oligosaccharides (i.e. soyoligosaccharides), gentiooligosaccharides, glucooligosaccharides, pecticoligosaccharides, palatinose polycondensates, difructose anhydride III, sorbitol, maltitol, lactitol, polyols, polydextrose, reduced paratinose, cellulose, β-glucose, β-galactose, β-fructose, verbascose, galactinol, and β-glucan, guar gum, pectin, high, sodium alginate, and lambda carrageenan, or mixtures thereof.

In one embodiment, a prebiotic component comprises a mixture of one of more of indigestible oligosaccharides, indigestible polysaccharides, free monosaccharides, digestible saccharides, starch, or non-starch polysaccharides.

In one embodiment a therapeutic composition comprising lactose further comprises a prebiotic component. In one embodiment the therapeutic composition comprising lactose further comprises 10% or less, by weight, of a prebiotic component. In one embodiment the therapeutic composition comprising lactose further comprises 0.1-10%, by weight, of a prebiotic component. In one embodiment the therapeutic composition comprising lactose further comprises 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9.2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, or 10%, by weight, of a prebiotic component. In one embodiment a therapeutic composition comprising lactose further comprises a weight of a prebiotic component about 0% to about 90%, about 10% to about 80%, about 20% to about 70%, about 30% to about 60%, about 40% to about 50%, about 0% to about 5%, about 5% to about 10%, about 10% to about 20%, about 20% to about 30%, about 30% to about 40%, about 40% to about 50%, about 50% to about 60%, about 60% to about 70%, about 70% to about 80%, or about 80% to about 90% of the weight of the therapeutic composition.

The term “about” means the referenced numeric indication plus or minus 10% of that referenced numeric indication.

The term “percent by weight,” as used in reference to the percent by weight of a component in a composition, means the percentage of the component's weight in comparison to the total dry weight of the composition.

### GOS

GOS (also known as galacto-oligosaccharides, galactooligosaccharides, trans-oligosaccharide (TOS), trans-galacto-oligosaccharide (TGOS), and trans-galactooligosaccharide) are oligomers or polymers of galactose molecules that generally terminate with a glucose molecule, or less often with a galactose molecule, and have varying degree of polymerization (generally the DP is between 2-20) and type of linkages. In one embodiment, GOS comprises galactose and glucose molecules. In another embodiment, GOS comprises only galactose molecules. In a further embodiment, GOS are galactose-containing oligosaccharides of the form of [β-D-Gal-(1-6)]ₙ-β-D-Gal-(1-4)-D-Glc wherein n is 1-20. In another embodiment, GOS are galactose-containing oligosaccharides of the form Glc α1-4-[β Gal 1-6]ₙ where n=1-20. In another embodiment, GOS are in the form of α-D-Glc (1-4)-[β-D-Gal-(1-6)-]ₙ where n=1-20. Gal is a galactopyranose unit and Glc (or Glu) is a glucopyranose unit. In one embodiment, a therapeutic composition comprises lactose and GOS. In another embodiment, a therapeutic composition comprises lactose and GOS in an enteric coated form. In another embodiment, a therapeutic composition comprises, consists essentially of, or consists of lactose and GOS in an enteric coated form.

In another embodiment, a therapeutic composition comprising lactose comprises a GOS-related compound. In another embodiment, a therapeutic composition comprises lactose and a GOS-related compound in an enteric coated form. In another embodiment, a therapeutic composition comprises, consists essentially of, or consists of lactose and a GOS-related compound in an enteric coated form. A GOS-related compound can have the following properties: a) a “lactose” moiety; e.g., classic GOS with a gal-glu moiety, of whatever polymerization value or type of linkage; or b) be stimulatory to “lactose fermenting” microbes in the human GI tract; for example, raffinose (gal-fru-glu) is a “related” GOS compound that is stimulatory to both Lactobacilli and Bifidobacteria.

In one embodiment, a therapeutic composition comprising lactose further comprises GOS with a low degree of polymerization. In one embodiment a therapeutic composition comprising lactose comprising GOS with a low degree of polymerization increases growth of probiotic bacteria to a greater extent than an equivalent amount of a therapeutic composition comprising lactose comprising GOS with a high degree of polymerization. In another embodiment, a therapeutic composition comprising lactose comprising a high percentage of GOS with a low degree of polymerization increases growth of probiotic bacteria to a greater extent than an equivalent amount of a lactose composition comprising a low percentage of GOS with a low degree of polymerization. In one embodiment a therapeutic composition comprising lactose further comprises GOS with a degree of polymerization less than 20, such as less than 10, less than 9, less than 8, less than 7, less than 6, less than 5, less than 4, or less than 3. In another embodiment a therapeutic composition comprising lactose further comprises GOS with a low degree of polymerization increases growth of probiotic bacteria in the gastrointestinal subject of a subject.

Linkages between the individual sugar units found in GOS include β-(1-6), β-(1-4), β-(1-3) and β-(1-2) linkages. β-(1-3) linkages are less common than β-(1-6), β-(1-4) linkages. In one embodiment, GOS comprises a number of β-(1-6) linked or β-(1-4) galactopyranosyl units linked to a terminal glucopyranosyl residue through an α-(1-4) glycosidic bond. In another embodiment, GOS comprises a number of β-(1-6) linked or β-(1-4) galactopyranosyl units linked to a terminal glucopyranosyl residue through an β-(1-4) glycosidic bond. In another embodiment, GOS formed by transgalactosylation comprise β-D-galactopyranosyl-(1-3) linkages. In one embodiment, GOS are branched saccharides. Branched oligosaccharides can be formed as an artifact of the transgalactosylation reaction. In another embodiment, GOS are linear saccharides.

In another embodiment, GOS is a mixture of oligosaccharides comprising 20-28% by weight of β (1-3) linkages, 20-25% by weight of β (1-4) linkages, and 45-55% by weight of β (1-6) linkages. In one embodiment, GOS is a mixture of oligosaccharides comprising 26% by weight of β (1-3) linkages, 23% by weight of β (1-4) linkages, and 51% by weight of β (1-6) linkages.

Alpha-GOS (also called alpha-bond GOS or alpha-linked GOS) are oligosaccharides having an alpha-galactopyranosyl group. Alpha-GOS comprises at least one alpha glycosidic linkage between the saccharide units. Alpha-GOS are generally represented by α-(Gal)ₙ (n usually represents an integer of 2 to 10) or α-(Gal)ₙGlc (n usually represents an integer of 1 to 9). Examples include a mixture of α-galactosylglucose, α-galactobiose, α-galactotriose, α-galactotetraose, and higher oligosaccharides. Additional non-limiting examples include melibiose, manninootriose, raffinose, stachyose and the like, which can be produced from beat, soy bean oligosaccharide and the like.

In one embodiment, a therapeutic composition comprising lactose further comprises GOS with alpha linkages and beta linkages.

### FOS or Inulin

In one embodiment, the prebiotic in a therapeutic composition comprising lactose is fructooligosaccharides (FOS). FOS are a non-digestible, soluble-fiber that supports the growth of beneficial bacteria in the intestinal tract, particularly two important strains—L. acidophilus and L. bifidus. These two strains play an essential role in reducing the number of pathogenic bacteria. Additional nutritional properties, such as the effect on colonic pH and stool bulking justify their classification as dietary fibers. In experimental models, it has also been reported that they improve the bioavailability of essential minerals. As a fiber, it is thought to slow digestion and allow the painless reintroduction of lactose into the body. FOS are chain oligomers or polymers of the sugar fructose that are found in a variety of foods. The sugar units can be linked in a single straight chain or can be a chain with side branches. In many cases small amounts of glucose are also contained in the chain. The length of the fructose chains can vary from source to source. FOS are primarily polyfructans with a degree of polymerization (DP) generally ranging from 2 to 20 (oligofructose) or greater than 20 (inulin). Generally, the D-fructose moieties in FOS are joined by (3-(2-1) linkages and the oligomers or polymers are terminated with a D-glucose molecule linked to fructose by an α-(1-2) bond.

In another embodiment, the prebiotic in a therapeutic composition comprising lactose is Inulin, which is an example of a longer chained compound that is considered a FOS. The shorter (lower molecular weight) compounds tend to have a sweet taste. The size and complexity of the FOS molecule gives it desirable characteristics. Although the simple sugars fructose and glucose are quickly absorbed into the body by the intestines, FOS for the most part is indigestible and therefore acts as a non-digestible fiber in the diet. This is because the human does not have the enzymes to break down the FOS as it travels down the digestive tract. When the FOS reaches the large intestine and the colon, the bacteria that are found there start to break down the FOS. These bacteria have the enzymes needed to break down FOS. Bifidobacteria have been reported to use FOS. It is believed that foods that promote bifidobacteria growth are good for the health.

In one embodiment a therapeutic composition comprising lactose further comprises a prebiotic component that is inulin, wherein the inulin comprises 40% or more of the component by weight, such as about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 89, 90, 91, 92, 93, 94, 95, 96, 97, 98, 99, or 100% inulin. In another embodiment the prebiotic component comprises 1-20 g of inulin, such as about 1, 2, 3, 4, 5, 6, 7, 8, 9, 10, 11, 12, 13, 14, 15, 16, 17, 18, 19, or 20 g of inulin. In another embodiment the prebiotic component comprises inulin, water or one or more digestible saccharides. In one embodiment the prebiotic component comprises less than about 10 ppm of a heavy metal (such as arsenic or lead), including but not limited to less than about 10, 9, 8, 7, 6, 5, 4, 3, 2, or 1 ppm of a heavy metal.

In one embodiment, a composition comprising FOS is administered to a subject in need thereof, prior to beginning treatment with a therapeutic composition comprising lactose. In one embodiment, a composition comprising FOS is co-administered with a therapeutic composition comprising lactose. In one embodiment, a therapeutic composition comprising lactose initially further comprises FOS for first administration to a subject in need thereof but does not comprise FOS for a second administration to a subject in need thereof. For example, the initial one, two, three, four, five, six, seven, eight, nine, ten, or more than ten days of treatment with a therapeutic composition comprising lactose can include FOS, with the use of FOS discontinued after that time. In one embodiment, FOS can be administered for the first two days of treatment, then the administration of FOS is discontinued. In one embodiment, FOS, either alone or in combination with other substances or treatments cab be administered after the treatment with lactose is terminated. The FOS can be administered for any suitable period after the termination of treatment with lactose, and can be administered daily or at regular or irregular intervals. Doses can be as described below.

Numerous FOS preparations are known in the art, and any suitable FOS preparation can be used in the methods and compositions disclosed herein. A composition comprising FOS can be administered in a dose from about 1 mg to about 10 gm, or about 1 mg to about 5 gm, or about 2 mg to about 1000 mg, or about 2 mg to about 500 mg, or about 2 mg to about 200 mg, or about 2 mg to about 100 mg, or about 2 mg to about 50 mg, or about 2 mg to about 20 mg, or about 5 mg to about 10 mg, or about 5, 6, 7, 7.5, 8, 9, or 10 mg. In one embodiment, a composition comprising FOS are used in a dose of about 7.5 mg. In one embodiment, a therapeutic composition comprising lactose further comprises FOS. In one embodiment, as the amount of lactose administered to a subject increases, the dose of FOS increases as well. For example, an initial dose of lactose can be about 0.6 to 1.0 gm, e.g., 0.8 gm, given in combination with about 5-10 mg, e.g., about 7.5 mg, of FOS. The dose of lactose can be increased incrementally by about 0.6 to 1.0 gm, e.g., 0.8 gm, and the accompanying dose of FOS can be increased by about 5-10 mg, e.g., about 7.5 mg, of FOS. In one embodiment, a therapeutic composition comprising lactose further comprises FOS. In another embodiment, a therapeutic composition comprises lactose and FOS in an enteric coated form. In another embodiment, a therapeutic composition comprises, consists essentially of, or consists of lactose and FOS in an enteric coated form. In another embodiment, a therapeutic composition comprising lactose further comprises Inulin. In another embodiment, a therapeutic composition comprises lactose and inulin in a enteric coated form. In another embodiment, a therapeutic composition comprises, consists essentially of, or consists of lactose and inulin in an enteric coated form.

### GOS and FOS

In another embodiment the prebiotic component comprises GOS and FOS. Both FOS and GOS are indigestible saccharides. β glycosidic linkages of saccharides, such as those found in, but not limited to, FOS and GOS, make these prebiotics generally indigestible and unabsorbable in the stomach and small intestine. Also, α-linked GOS (α-GOS) was not hydrolyzed by human salivary amylase, and can be used by Bifidobacterium bifidum and Clostridium butyricum (Yamashita A. et al. (2004) J. Appl. Glycosci. 51:115-122). FOS and GOS pass through to the large intestine (colon) mostly intact where they are broken down and metabolized by various probiotics.

In one embodiment, a therapeutic composition comprising lactose further comprises a prebiotic component that comprises GOS and FOS. In another embodiment, a therapeutic composition comprises lactose, FOS and GOS in an enteric coated form. In another embodiment, a therapeutic composition comprises, consists essentially of, or consists of lactose, FOS and GOS in an enteric coated form. In one embodiment, 90% by weight of the prebiotic component is GOS and 10% by weight of the prebiotic component is FOS. In one embodiment, 50% by weight of the prebiotic component is GOS and 50% by weight of the prebiotic component is FOS. In one embodiment, 40-90% by weight of the prebiotic component is GOS and 10-60% by weight of the prebiotic component is FOS. In another embodiment, the prebiotic component of a therapeutic composition comprising lactose is 90-100% by weight GOS.

### Lactulose

Lactulose is a disaccharide that is formed from one molecule of fructose and galactose. It can be produced by isomerization of lactose. In one embodiment the prebiotic component comprises lactulose (4-O-β-D-Galactopyranosyl-β-D-fructofuranose), wherein the lactulose comprises about 40% or more of the composition by weight, such as about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 89, 90, 91, 92, 93, 94, 95, 96, 97, 98, 99, or 100% lactulose. In another embodiment the prebiotic component comprises 1-20 g of lactulose, such as about 1, 2, 3, 4, 5, 6, 7, 8, 9, 10, 11, 12, 13, 14, 15, 16, 17, 18, 19, or 20 g of lactulose. In another embodiment the prebiotic component comprises lactulose, water, or one or more digestible saccharides. In one embodiment the prebiotic component comprises less than about 10 ppm of a heavy metal (such as arsenic or lead), including but not limited to less than about 10, 9, 8, 7, 6, 5, 4, 3, 2, or 1 ppm of a heavy metal. In one embodiment, a therapeutic composition comprising lactose further comprises lactulose. In another embodiment, a therapeutic composition comprises lactose and lactulose in an enteric coated form. In one embodiment, a therapeutic composition comprises, consists essentially of, or consists of lactose and lactulose in an enteric coated form.

### Raffinose

Raffinose (melitose, melitriose, gossypose, α-D-galactosylsucrose) is a trisaccharide composed of galactose, fructose, and glucose. The enzyme α-galactosidase, which is not found in the human digestive tract, can hydrolyze raffinose. Thus, in humans, raffinose passes through the stomach and upper intestine and is digested by bacteria that do contain α-galactosidase in the lower intestine. In one embodiment the prebiotic component comprises raffinose, wherein the raffinose comprises 40% or more of the composition by weight, such as about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 89, 90, 91, 92, 93, 94, 95, 96, 97, 98, 99, or 100% raffinose. In another embodiment the prebiotic component comprises 1-20 g of raffinose, such as about 1, 2, 3, 4, 5, 6, 7, 8, 9, 10, 11, 12, 13, 14, 15, 16, 17, 18, 19, or 20 g of raffinose. In another embodiment the prebiotic component comprises raffinose, or one or more digestible saccharides. In one embodiment the prebiotic component comprises less than about 10 ppm of a heavy metal (such as arsenic or lead), including but not limited to less than about 10, 9, 8, 7, 6, 5, 4, 3, 2, or 1 ppm of a heavy metal. In one embodiment, a therapeutic composition comprising lactose further comprises raffinose. In another embodiment, a therapeutic composition comprises lactose and raffinose in a enteric coated form. In another embodiment, a therapeutic composition comprises, consists essentially of, or consists of lactose and raffinose in an enteric coated form.

### Stachyose

Stachyose is a tetrasaccharide that consists of two α-D-galactose units, one α-D-glucose unit, and one β-D-fructose unit. It is linked as gal(α1→6) gal(α1→6)glc(α1 [symbol] 2β)fru. Stachyose is not completely digestible by humans. In one embodiment the prebiotic component comprises stachyose, wherein the stachyose comprises 40% or more of the composition by weight, such as about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 89, 90, 91, 92, 93, 94, 95, 96, 97, 98, 99, or 100% stachyose. In another embodiment the prebiotic component comprises 1-20 g of stachyose, such as about 1, 2, 3, 4, 5, 6, 7, 8, 9, 10, 11, 12, 13, 14, 15, 16, 17, 18, 19, or 20 g of stachyose. In another embodiment the prebiotic component comprises stachyose, water, or one or more digestible saccharides. In one embodiment the prebiotic component comprises less than about 10 ppm of a heavy metal (such as arsenic), including but not limited to less than about 10, 9, 8, 7, 6, 5, 4, 3, 2, or 1 ppm of a heavy metal. In one embodiment, a therapeutic composition comprising lactose further comprises stachyose. In another embodiment, a therapeutic composition comprises lactose and stachyose in an enteric coated form. In one embodiment, a therapeutic composition comprises, consists essentially of, or consists of lactose and stachyose in an enteric coated form.

In one embodiment wherein a therapeutic composition comprising lactose comprises a prebiotic component, the prebiotic component comprises one or more saccharides (herein, interchangeably also referred to as carbohydrate or sugar) which are indigestible by a human digestive system. In another embodiment the prebiotic component consists essentially of a saccharide which is indigestible by a human digestive system. In one embodiment, the one or more saccharides are oligosaccharides wherein the degree of polymerization is from 2 to 20. In one embodiment the degree of polymerization can be 2, 3, 4, 5, 6, 7, 8, 9, 10, 11, 12, 13, 14, 15, 16, 17, 18, 19, or 20. In another embodiment, the one or more saccharides are a polysaccharide wherein the degree of polymerization is greater than 10. In another embodiment, the saccharide comprises a mixture of indigestible oligosaccharides or polysaccharides. In another embodiment the prebiotic component comprises one or more digestible saccharides and one or more indigestible oligosaccharides or polysaccharides. In one embodiment the saccharide is an oligosaccharide, such as a disaccharide, a trisaccharide, a tetrasaccharide, a pentasaccharide, a hexasaccharide, a heptasaccharide, an octasaccharide, a nanasaccharide, or a decasaccharide. Saccharides that are not digestible by humans include, but are not limited to, transgalactooligosaccharides, galacto-oligosaccharides, lactulose, raffinose, stachyose, lactosucrose, fructo-oligosaccharides, isomalto-oligosaccharides, xylo-oligosaccharides, paratinose oligosaccharides, difructose anhydride III, sorbitol, maltitol, lactitol, reduced paratinose, cellulose, β-glucose, β-galactose, β-fructose, verbascose, galactinol, and β-glucan, guar gum, pectin, high sodium alginate, and lambda carrageenan.

In one embodiment the prebiotic component comprises a saccharide that is inulin, fructo-oligosaccharide (FOS), lactulose, galacto-oligosaccharide (GOS), raffinose, or stachyose. In another embodiment the saccharide that is an oligosaccharide that is indigestible by a human digestive system and contains at least one beta-glycosidic (e.g., beta galactosidic or beta glucosidic) bond that when fed to a subject in need thereof would facilitate lactose digestion. In one embodiment the subject in need thereof is a human. In another embodiment the saccharide is an oligosaccharide that is indigestible by a human digestive system and contains at least one beta-glycosidic (e.g., beta galactosidic or beta glucosidic) bond that can be digested by a bacteria. In one embodiment the bacteria is a probiotic. In one embodiment the saccharide is an oligosaccharide that is indigestible by a human digestive system and contains at least one alpha-glycosidic bond. In one embodiment the bacteria is a lactobacillus or a bifidobacteria. In one embodiment the saccharide is GOS.

In another embodiment the saccharide is an oligosaccharide that is indigestible by a human digestive system and contains at least one alpha-glycosidic (e.g., alpha galactosidic or alpha glucosidic) bond that when fed to a subject in need thereof would induce lactose digestion. In one embodiment the subject in need thereof is a human. In another embodiment the saccharide is an oligosaccharide that is indigestible by a human digestive system and contains at least one alpha-glycosidic (e.g., alpha galactosidic or alpha glucosidic) bond that can be metabolized by a bacterium. In one embodiment the bacteria is a probiotic. In one embodiment the bacterium is a lactobacillus or a bifidobacteria. In another embodiment, a therapeutic composition comprising lactose contains at least one strain of probiotic bacteria.

In one embodiment, the prebiotic component contains an oligosaccharide that increases β-galatosidase activity in the large intestine. In one embodiment, the prebiotic component contains an oligosaccharide that increases the amount of probiotic activity in the large intestine.

In one embodiment, a therapeutic composition comprising lactose further comprises a probiotic or prebiotic component. In another embodiment, a therapeutic composition comprising lactose is in the form of a powder, tablet, capsule, or liquid. In one embodiment, a therapeutic composition comprising lactose is formulated to be administered with a dairy product such as milk, yogurt, shake, smoothie, cheese, ice cream and the like.

In embodiments where a therapeutic composition comprising lactose comprises less than 100% by weight of lactose the remaining ingredients can be any suitable ingredients intended for the consumption of the subject in need thereof, e.g., human, including, but not limited to, a prebiotic (e.g., FOS), a buffer, one or more other digestible saccharides, ingredients intended to inhibit clumping and increase pourability, such as silicone dioxide and microcrystalline cellulose, or similar ingredients as are well-known in the art. Remaining ingredients can also include ingredients to improve handling, preservatives, antioxidants, flavorings and the like.

### Buffer Components

One or more buffers, optionally with a calcium counterion, can also be administered in methods and compositions described herein. Any buffer suitable for consumption by the subject being treated, e.g., human, are useful for the compositions herein. The buffer neutralizes stomach acidity which can, e.g., allow live bacteria to reach the gut. Buffers include citrates, phosphates, and the like. One embodiment utilizes a buffer with a calcium counterion, such as Calcium Phosphate Tribasic. The calcium can serve to restore the calcium that many lactose intolerant subjects are missing in their diet. A recent study demonstrated the ability of calcium phosphate to protect Lactobacillus acidophilus from bile. It is an excellent buffering agent and will help neutralize stomach acidity.

In one embodiment, a buffer such as calcium phosphate is given prior to beginning treatment with lactose, optionally in conjunction with administration of bacteria. In one embodiment, a therapeutic composition comprising lactose further comprises a buffer, such as calcium phosphate. Thus, in one embodiment, some or all doses of a therapeutic composition comprising lactose are accompanied by a dose of a buffer such as calcium phosphate. In one embodiment, a buffer such as calcium phosphate is given initially with a therapeutic composition comprising lactose but then its use is discontinued. For example, the initial one, two, three, four, five, six, seven, eight, nine, ten, or more than ten days of treatment with lactose can include doses of a buffer such as calcium phosphate, with the use of the buffer discontinued after that time. In one embodiment, a buffer such as calcium phosphate can be given for the first two days of treatment with a therapeutic composition comprising lactose, then the administration of buffer is discontinued. In one embodiment, a buffer such as calcium phosphate, either alone or in combination with other substances or treatments is used after the treatment with a therapeutic composition comprising lactose is terminated. A buffer such as calcium phosphate can be administered for any suitable period after the termination of treatment with lactose, and can be administered daily or at regular or irregular intervals. Doses can be as described below.

Numerous buffers suitable for human consumption are known in the art, and any suitable buffer can be used in the methods and compositions described herein. Calcium triphosphate is an exemplary buffer and has the advantage that its counterion supplies a nutrient that is often lacking in lactose-intolerant subjects, i.e. calcium. The buffer can be used in a dose from about 2 mg to about 2000 mg, or about 4 mg to about 400 mg, or about 4 mg to about 200 mg, or about 4 mg to about 100 mg, or about 8 mg to about 50 mg, or about 10 mg to about 40 mg, or about 20 mg to about 30 mg, or about 20, 21, 22, 23, 24, 25, 26, 27, 28, 29, or 30 mg. In one embodiment, buffer is used in a dose of about 25 mg. In one embodiment, calcium phosphate is used in a dose of about 25 mg. The dose can be given in combination with lactose. In one embodiment, as lactose dose increases, the dose of buffer increases as well. For example, an initial dose of lactose can be about 0.6 g to 1.0 g, e.g., 0.8 g, given in combination with about 20-30 mg, e.g., about 25 mg, of buffer, e.g., calcium phosphate. The dose of lactose can be increased incrementally by about 0.6 g to 1.0 g, e.g., 0.8 g, and the accompanying dose of buffer, e.g., calcium phosphate, can be increased by about 20-30 mg, e.g., about 25 mg, of buffer, e.g., calcium phosphate.

### Compositions and Formulations

In one aspect a therapeutic composition comprising lactose for the treatment of the symptoms of lactose intolerance is provided. In one embodiment a lactose composition consists essentially of lactose. In one embodiment a lactose composition consists of lactose. In another embodiment the lactose composition comprises lactose, a probiotic component, and essentially no prebiotic component. In another embodiment a lactose composition contains lactose, a probiotic component, and a prebiotic component, wherein the probiotic component comprises at least one probiotic bacteria strain. In another embodiment a lactose composition comprises a prebiotic component, a probiotic component, and a buffer component. In one embodiment, a lactose composition comprises lactose, a prebiotic and no probiotic. Additional ingredients include ingredients to improve handling, preservatives, antioxidants, flavorings and the like.

In another embodiment, a therapeutic composition comprising lactose is in the form of a powder, tablet, capsule, enteric coated capsule, or liquid. In one embodiment, a therapeutic composition comprising lactose can be administered with a dairy product and is in the form of milk or other common dairy product such as a yogurt, shake, smoothie, cheese, and the like.

In embodiments where a therapeutic composition comprising lactose comprises less than 100% by weight of lactose the remaining ingredients can be any suitable ingredients intended for the consumption of the subject in need thereof, e.g., human, including, but not limited to, a prebiotic (e.g., FOS), a buffer, one or more digestible saccharides, ingredients intended to inhibit clumping and increase pourability, such as silicone dioxide and microcrystalline cellulose, or similar ingredients as are well-known in the art. Remaining ingredients can also include ingredients to improve handling, preservatives, antioxidants, flavorings and the like.

### Therapeutic Compositions Comprising Lactose

In one embodiment, a therapeutic composition comprising lactose does not comprise a prebiotic component. In another embodiment, a therapeutic composition comprising lactose does not comprise a probiotic component. In another embodiment, a therapeutic composition comprising lactose does not comprise a probiotic component and a prebiotic component. In another embodiment, a therapeutic composition comprising lactose does not comprise a buffer. In another embodiment, a therapeutic composition comprising lactose does not comprise a probiotic component and a buffer. In another embodiment, a therapeutic composition comprising lactose does not comprise a prebiotic component and a buffer. In another embodiment, a therapeutic composition comprising lactose does not comprise a probiotic component, a prebiotic component and a buffer. In another embodiment, a therapeutic composition consists essentially of lactose. In another embodiment, a therapeutic composition consists of lactose.

In another embodiment, a therapeutic composition comprising lactose is formulated for release in a subject's intestines. In another embodiment, a therapeutic composition comprising lactose is formulated for release in a subject's lower intestines. In one embodiment the therapeutic composition comprising lactose is formulated for controlled release. In one embodiment the therapeutic composition comprising lactose further comprises an enteric coating.

In another embodiment, a therapeutic composition consisting essentially of or consisting of lactose is formulated for release in a subject's intestines. In another embodiment, a therapeutic composition consisting essentially of or consisting of lactose is formulated for release in a subject's lower intestines. In one embodiment the therapeutic composition consisting essentially of, or consisting of lactose is formulated for controlled release. In one embodiment the therapeutic composition consisting essentially of lactose further comprises an enteric coating. In one embodiment, a therapeutic composition comprising, consisting essentially of, or consisting of lactose is in the form of a capsule or sachet.

### Compositions Comprising Lactose and at Least One Probiotic Bacteria Strain

In one embodiment, a therapeutic composition comprising lactose further comprises a probiotic component, wherein the probiotic component comprises at least one probiotic bacteria strain. The lactose can comprise more than 50% of the weight of a therapeutic composition comprising lactose while the at least one probiotic bacteria strain will typically comprise less than about 10%, 5%, 4%, 3%, or 2% by weight of the compositions (herein all percentages are weight percent unless otherwise indicated). For example, the lactose can be present at about 70-99.75% by weight and the at least one probiotic bacteria strain at about 0.1% to about 50%, about 0.1% to about 40%, about 0.1% to about 30%, about 0.1% to about 20%, about 0.1% to about 10%, about 0.1% to about 5%, or about 0.25-2% by weight, or the lactose can be present at about 89-96% by weight and the bacteria at about 1.2-3.7% by weight. In one embodiment, lactose is present at about 92% by weight and at least one probiotic bacteria strain, is present at about 1.5% by weight. In one embodiment, lactose is present at about 92% by weight and at least one probiotic bacteria strain, is present at about 1.5% by weight. In another embodiment, lactose is present at about 93% by weight and at least one probiotic bacteria strain, is present at about 1.5% by weight. In another embodiment, lactose is present at about 94% by weight and at least one probiotic bacteria strain, is present at about 1.5% by weight. In another embodiment, lactose is present at about 95% by weight and at least one probiotic bacteria strain is present at about 1.5% by weight. In another embodiment, lactose is present at about 96% by weight and at least one probiotic bacteria strain is present at about 1.5% by weight. In another embodiment, lactose is present at about 97% by weight and at least one probiotic bacteria strain is present at about 1.5% by weight. In another embodiment, lactose is present at about 98% by weight and at least one probiotic bacteria strain is present at about 1.5% by weight. In another embodiment, lactose is present at about 98.5% by weight and at least one probiotic bacteria strain is present at about 1.5% by weight. In one embodiment the probiotic bacteria strain is L. acidophilus or a bifidobacteria strain.

In one embodiment, a therapeutic composition comprising, consisting essentially of, or consisting of lactose and a probiotic is formulated for controlled release such as by formulation with an enteric coating. If the at least one probiotic bacteria strain and the lactose do not make up 100% by weight of the prebiotic composition, the remaining ingredients can be any suitable ingredients intended for the consumption by a subject in need thereof, including, but not limited to, other prebiotics (e.g., FOS), or one or more buffers. In another embodiment a therapeutic composition further comprises one or more excipients such as a digestible saccharide, silicone dioxide, microcrystalline cellulose, or similar ingredients known in the art. Remaining ingredients can also include ingredients to improve handling, preservatives, antioxidants, flavorings or the like. In one embodiment, a therapeutic composition comprising, consisting essentially of, or consisting of lactose and a probiotic is in the form of a capsule or sachet.

### Compositions Comprising Lactose and at Least One Prebiotic

In one embodiment, a therapeutic composition comprises, consists essentially of, or consists of lactose and at least one prebiotic. In one embodiment, the prebiotic is GOS or FOS. In one embodiment, lactose can be present at about 80 to about 99.9% by weight and a prebiotic at about 0.10-20% by weight, or the lactose can be present at about 89 to about 94% by weight and the prebiotic at about 0.40 to about 11% by weight. In one embodiment, the prebiotic is present at about 0.10 to about 1.89% by weight, or about 0.40 to about 1.26% by weight. In one embodiment, lactose is present at about 94.01% and prebiotic is present at about 0.88% by weight. In another embodiment, lactose is present at about 50% to about 99.9% and a prebiotic at about 0.1% to about 50%, about 0.2% to about 50%, about 0.3% to about 50%, about 0.4% to about 50%, about 0.5% to about 50%, about 0.6% to about 50%, about 0.7% to about 50%, about 0.8% to about 50%, about 0.9% to about 50%, about 1% to about 50%, about 0.1% to about 1%, about 1% to about 2%, about 2% to about 5%, about 5% to about 10%, about 10% to about 20%, about 20% to about 30%, about 30% to about 40%, or about 40% to about 50 by weight. In another embodiment, lactose is present at about 50% to about 99.9% and a prebiotic at more than about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40%, or 45%.

In one embodiment, a therapeutic composition comprising, consisting essentially of, or consisting of lactose and a prebiotic is formulated for controlled release such as formulation with an enteric coating. If the prebiotic and lactose do not make up 100% of the composition, the remaining ingredients can be any suitable ingredients intended for the consumption by an individual, e.g., human, including, but not limited to, other prebiotics, bacteria and/or buffer, but also including ingredients intended to inhibit clumping and increase pourability, such as silicone dioxide and microcyrstalline cellulose, or similar ingredients as are well-known in the art. In one embodiment, a therapeutic composition comprising, consisting essentially of, or consisting of lactose and a prebiotic is in the form of a capsule or sachet.

### Compositions Comprising Lactose and a Buffer

In one embodiment, a therapeutic composition comprises, consists essentially of, or consists of lactose and a buffer component, wherein the buffer component comprises at least one buffer. In another embodiment, a lactose composition comprises lactose and a buffer (e.g., calcium phosphate tribasic). For example, lactose can be present at about 60-100% by weight and the buffer at about 0.50-5% by weight, or the lactose can be present at about 70-96% by weight and the buffer at about 1 to about 4% by weight. In one embodiment, lactose can be present at about 50 to about 99.9% by weight and a buffer at about 0.5% to about 50%, about 1% to about 50%, about 2% to about 50%, about 3% to about 50%, about 4% to about 50%, about 5% to about 50%, about 0.5% to about 1%, about 0.5% to about 5%, about 1% to about 5%, about 5% to about 10%, about 10% to about 20%, about 20% to about 30%, about 30% to about 40%, or about 40% to about 50%. In one embodiment, lactose is present at about 90% by weight and buffer is present at about 3% by weight. In another embodiment, lactose is present at about 92% by weight and buffer is present at about 3% by weight. In another embodiment, lactose is present at about 93% by weight and buffer is present at about 3% by weight. In another embodiment, lactose is present at about 94% by weight and buffer is present at about 3% by weight. In another embodiment, lactose is present at about 95% by weight and buffer is present at about 3% by weight. In another embodiment, lactose is present at about 96% by weight and buffer is present at about 3% by weight. In another embodiment, lactose is present at about 97% by weight and buffer is present at about 2% by weight. In another embodiment, lactose is present at about 98% by weight and buffer is present at about 1% by weight. In another embodiment, lactose is present at about 99% by weight and buffer is present at about 1% by weight. In another embodiment, lactose is present at about 100% by weight and buffer is present at less than about 1% by weight.

In one embodiment, a therapeutic composition comprises, consists essentially of, or consists of lactose and a buffer component, is formulated for controlled release of the therapeutic composition. In another embodiment the therapeutic composition is formulated with an enteric coating. If the buffer and lactose do not make up 100% by weight of the composition, the remaining ingredients can be any suitable ingredients intended for the consumption of the subject (e.g., a human) including, but not limited to, probiotics (e.g., beneficial bacteria) or other prebiotics (e.g., FOS), but also including pharmaceutically acceptable ingredients, such as silicone dioxide and microcrystalline cellulose, or similar ingredients as are well-known in the art. Pharmaceutically acceptable ingredients can also include ingredients to improve handling, preservatives, antioxidants, flavorings and the like. In one embodiment, a therapeutic composition comprises, consists essentially of, or consists of lactose and a buffer component, and is in the form of a capsule or sachet.

### Compositions Comprising Lactose, a Prebiotic, and a Probiotic Bacteria

In one embodiment, a therapeutic composition comprises, consists essentially of, or consists of lactose, a prebiotic component, and a probiotic component, wherein the prebiotic component comprises at least one prebiotic and the probiotic component comprises at least one probiotic bacteria strain. In one embodiment, the compositions contain lactose, a probiotic bacteria (e.g., L. acidophilus or bifidobacteria), and a prebiotic (e.g., FOS or GOS). For example, lactose can be present at about 60-99.75% by weight, bacteria at about 0.25-2.10% by weight and the prebiotic at about 0.10-1.89% by weight, or the lactose can be present at about 89-94% by weight, bacteria at about 0.91-1.95% by weight and the prebiotic at about 0.40 to about 1.26% by weight. In one embodiment, lactose can be present at about 50 to about 99.75% by weight, bacteria at about 0.1% to about 50% by weight and the prebiotic at about 0.1% to about 50% by weight. The bacteria can be present at about 0.1% to about 1%, about 0.1% to about 2%, about 0.1% to about 5%, about 1% to about 10%, about 10% to about 20%, about 20% to about 30%, about 30% to about 40%, about 40% to about 50%. The prebiotic can be present at about 0.1% to about 1%, about 0.1% to about 2%, about 0.1% to about 5%, about 1% to about 10%, about 10% to about 20%, about 20% to about 30%, about 30% to about 40%, or about 40% to about 50%. In one embodiment, lactose is present at about 94.01% by weight, bacteria at about 1.47% by weight, and prebiotic is present at about 0.88% by weight. In one embodiment, the prebiotic can be present at about 1-20% by weight, bacteria at about 0.25-2.10% by weight, and the lactose at about 70-98.75% by weight. In another embodiment the prebiotic can be present at about 5-20% by weight, bacteria at about 0.91-1.95% by weight and the lactose at about 70 to about 96% by weight. In another embodiment, prebiotic is present at about 20% by weight, bacteria at about 1.5% by weight, and lactose is present at about 70% by weight. In another embodiment, prebiotic is present at about 5% by weight, bacteria at about 1.5% by weight, and lactose is present at about 90% by weight. In another embodiment, prebiotic is present at about 5% by weight, bacteria at about 1.5% by weight, and lactose is present at about 92% by weight. In another embodiment, prebiotic is present at about 5% by weight, bacteria at about 1.5% by weight, and lactose is present at about 93% by weight. In another embodiment, prebiotic is present at about 5% by weight, bacteria at about 1% by weight, and lactose is present at about 94% by weight. In another embodiment, prebiotic is present at about 4.5% by weight, bacteria at about 1.5% by weight, and lactose is present at about 94% by weight. In another embodiment, prebiotic is present at about 4.5% by weight, bacteria at about 0.5% by weight, and lactose is present at about 95% by weight. In another embodiment, prebiotic is present at about 3.5% by weight, bacteria at about 0.5% by weight, and lactose is present at about 96% by weight. In another embodiment, prebiotic is present at about 2.5% by weight, bacteria at about 0.5% by weight, and lactose is present at about 97% by weight. In another embodiment, prebiotic is present at about 1.5% by weight, bacteria at about 0.5% by weight, and lactose is present at about 98% by weight. In another embodiment, prebiotic is present at about 0.5% by weight, bacteria at about 0.5% by weight, and lactose is present at about 99% by weight.

In one embodiment, a therapeutic composition comprises, consists essentially of, or consists of lactose, a prebiotic component, and a probiotic component, wherein the prebiotic component comprises at least one prebiotic and the probiotic component comprises at least one probiotic bacteria strain, and is formulated for controlled release of the therapeutic composition. In another embodiment the therapeutic composition is formulated with an enteric coating. If the probiotic bacteria, lactose and prebiotic do not make up 100% of the composition, the remaining ingredients can be any suitable ingredients intended for the consumption of the subject, e.g., a human, including, but not limited to a buffer, other digestible saccharides (e.g., glucose, or galactose), ingredients intended to inhibit clumping and increase pourability, such as silicone dioxide and microcrystalline cellulose, or other pharmaceutically acceptable ingredients known in the art. Remaining ingredients can also include ingredients to improve handling, preservatives, antioxidants, flavorings and the like. In one embodiment, a therapeutic composition comprises, consists essentially of, or consists of lactose, a prebiotic component, and a probiotic component, wherein the therapeutic composition is in the form of a pill, tablet, caplet, capsule or sachet. In one embodiment the pill, tablet, caplet or capsule is formulated with an enteric coating.

### Compositions Comprising Lactose, a Probiotic Bacteria, and Buffer

In one embodiment, a therapeutic composition comprises, consists essentially of, or consists of lactose, a probiotic component, and a buffer component, wherein the probiotic component comprises at least one probiotic strain and the buffer component comprises at least one buffer. In one embodiment, the compositions contain lactose, bacteria, and buffer. For example, lactose can be present at about 20-99.25%, bacteria at about 0.25-2.10%, and the buffer at about 0.50-4%, or the lactose can be present at about 89-94%, bacteria at about 0.91-1.95% and the buffer at about 1.2 to about 3.75%. In one embodiment, lactose is present at about 94.01%, bacteria at about 1.47%, and buffer is present at about 2.94%. In another embodiment, lactose is present at about 20-99.25% by weight, a probiotic bacteria strain at about 0.25-10% by weight, and the buffer at about 0.50-20% by weight. In another embodiment, lactose can be present at about 70-95% by weight, a probiotic bacteria strain at about 0.91-1.95% by weight and the buffer at about 1.2-3.75% by weight. In another embodiment, lactose is present at about 70% by weight, a probiotic bacteria strain at about 1.5% by weight, and buffer is present at about 3% by weight. In another embodiment, lactose is present at about 90% by weight, a probiotic bacteria strain at about 1.5% by weight, and buffer is present at about 3% by weight. In another embodiment, lactose is present at about 92% by weight, a probiotic bacteria strain at about 1.5% by weight, and buffer is present at about 3% by weight. In another embodiment, lactose is present at about 93% by weight, a probiotic bacteria strain at about 1.5% by weight, and buffer is present at about 3% by weight. In another embodiment, lactose is present at about 94% by weight, a probiotic bacteria strain at about 1.5% by weight, and buffer is present at about 3% by weight. In another embodiment, lactose is present at about 95% by weight, a probiotic bacteria strain at about 1.5% by weight, and buffer is present at about 3% by weight. In another embodiment, lactose is present at about 96% by weight, a probiotic bacteria strain at about 1.5% by weight, and buffer is present at about 2% by weight. In another embodiment, lactose is present at about 97% by weight, a probiotic bacteria strain at about 1.5% by weight, and buffer is present at about 1.5% by weight. In another embodiment, lactose is present at about 99% by weight, a probiotic bacteria strain at about 0.5% by weight, and buffer is present at about 0.5% by weight. In another embodiment, lactose is present at about 99.9% by weight, a probiotic bacteria strain at less than about 0.5% by weight, and buffer is present at less than about 0.5% by weight.

In one embodiment, a therapeutic composition comprises, consists essentially of, or consists of lactose, a probiotic component, and a buffer component, wherein the probiotic component comprises at least one probiotic strain and the buffer component comprises at least one buffer and is formulated for controlled release of the therapeutic composition or formulated with enteric coating. If the a probiotic bacteria strain, buffer and lactose do not make up 100% of the composition, the remaining ingredients can be any suitable ingredients intended for the consumption of a subject (e.g., human) including, but not limited to, other probiotics, other digestible saccharides (e.g., lactose, glucose or galactose), ingredients intended to inhibit clumping and increase pourability, such as silicone dioxide and microcrystalline cellulose, or similar ingredients as are well-known in the art. Remaining ingredients can also include ingredients to improve handling, preservatives, antioxidants, flavorings and the like. In one embodiment, a therapeutic composition comprises, consists essentially of, or consists of lactose, a probiotic component and a buffer component, and is in the form of a pill, tablet, caplet, capsule or sachet. In one embodiment the pill, tablet, caplet or capsule is formulated with an enteric coating.

### Compositions Comprising a Prebiotic, Lactose, and a Buffer

In one embodiment, a therapeutic composition comprises, consists essentially of, or consists of lactose, a prebiotic component (e.g., FOS or GOS), and a buffer component, wherein the prebiotic component comprises at least one prebiotic and the buffer component comprises at least one buffer. In one embodiment, the compositions contain lactose, prebiotic, and buffer. For example, lactose can be present at about 60-99.75%, prebiotic at about 0.10 to about 1.89%, and the buffer at about 0.50-4%, or the lactose can be present at about 89-94%, prebiotic at about 0.40 to about 1.26%, and the buffer at about 1.2 to about 3.75%. In one embodiment, lactose can be present at about 50% to about 99.75%, prebiotic at about 0.1% to about 50%, and buffer at about 0.1% to about 50%. In one embodiment, lactose can be present at about 50% to about 99.75%; the prebiotic can be present at about 0.1% to about 1%, about 0.1% to about 2%, about 0.1% to about 3%, about 0.1% to about 4%, about 0.1% to about 5%, about 1% to about 2%, about 2% to about 3%, about 3% to about 4%, about 4% to about 5%, about 5% to about 10%, about 10% to about 20%, about 20% to about 30%, about 30% to about 40%, or about 40% to about 50%; and the buffer can be present at about 0.1% to about 1%, about 1% to about 2%, about 2% to about 3%, about 3% to about 4%, about 4% to about 5%, about 0.1% to about 5%, about 5% to about 10%, about 10% to about 20%, about 20% to about 30%, about 30% to about 40%, or about 40% to about 50%. In one embodiment, lactose is present at about 94.01%, prebiotic at about 0.88%, and buffer is present at about 2.94%. If the prebiotic, buffer and lactose do not make up 100% of the composition, the remaining ingredients can be any suitable ingredients intended for the consumption by the individual, e.g., human, including, but not limited to, bacteria, but also including ingredients intended to inhibit clumping and increase pourability, such as silicone dioxide and microcyrstalline cellulose, or similar ingredients as are well-known in the art. For example, prebiotic can be present at about 1-20% by weight, lactose at about 60-100% by weight, and the buffer at about 0.50-4% by weight, or the prebiotic can be present at about 5-20% by weight, lactose at about 70-96% by weight, and the buffer at about 1.2-3.75% by weight. In one embodiment, prebiotic is present at about 20% by weight, lactose at about 70% by weight, and buffer is present at about 3% by weight. In another embodiment, prebiotic is present at about 5% by weight, lactose at about 90% by weight, and buffer is present at about 3% by weight. In another embodiment, prebiotic is present at about 5% by weight, lactose at about 92% by weight, and buffer is present at about 3% by weight. In another embodiment, prebiotic is present at about 4% by weight, lactose at about 93% by weight, and buffer is present at about 3% by weight. In another embodiment, prebiotic is present at about 3% by weight, lactose at about 94% by weight, and buffer is present at about 3% by weight. In another embodiment, prebiotic is present at about 2% by weight, lactose at about 95% by weight, and buffer is present at about 3% by weight. In another embodiment, prebiotic is present at about 1% by weight, lactose at about 96% by weight, and buffer is present at about 3% by weight.

In one embodiment, a therapeutic composition comprises, consists essentially of, or consists of lactose, a prebiotic component, and a buffer component, wherein the prebiotic component comprises at least one prebiotic and the buffer component comprises at least one buffer and is formulated for controlled release of the therapeutic composition. In another embodiment the therapeutic composition is formulated with an enteric coating. If the lactose, buffer and prebiotic do not make up 100% of the composition by weight, the remaining ingredients can be any suitable ingredients intended for the consumption of a subject (e.g., human) including, but not limited to, bacteria, ingredients intended to inhibit clumping and increase pourability, such as silicone dioxide and microcrystalline cellulose, or similar ingredients as are well-known in the art. Remaining ingredients can also include ingredients to improve handling, preservatives, antioxidants, flavorings and the like. In one embodiment, a therapeutic composition comprises, consists essentially of, or consists of lactose, a prebiotic component and a buffer is in the form of a pill, tablet, caplet, capsule or sachet. In one embodiment the pill, tablet, caplet or capsule is formulated with an enteric coating.

### Compositions Comprising a Prebiotic, Bacteria, Lactose, and a Buffer

In one embodiment, a therapeutic composition comprises, consists essentially of, or consists of lactose, a probiotic component, a prebiotic component (e.g., FOS), and a buffer component, wherein the probiotic component comprises at least one probiotic strain, the prebiotic component comprises at least one prebiotic and the buffer component comprises at least one buffer. In one embodiment, the compositions contain lactose, bacteria, prebiotic, and buffer. For example, lactose can be present at about 80-99.75%, bacteria at about 0.25 to about 2.10%, prebiotic at about 0.10 to about 1.89%, and the buffer at about 0.50-4%, or the lactose can be present at about 89-94%, bacteria at about 0.91 to about 1.95%, prebiotic at about 0.40 to about 1.26%, and the buffer at about 1.2 to about 3.75%. In one embodiment, lactose is present at about 94.01%, bacteria at about 1.47%, prebiotic at about 0.88%, and buffer is present at about 2.94%. In another example, lactose can be present at about 1-20% by weight, bacteria at about 0.25-2.10% by weight, lactose at about 60-100% by weight, and the buffer at about 0.50-4% by weight, or the lactose can be present at about 5-20% by weight, bacteria at about 0.91-1.95% by weight, lactose at about 70-95% by weight, and the buffer at about 1.2-3.75% by weight. In one embodiment, lactose is present at about 20% by weight, bacteria at about 1.47% by weight, lactose at about 70% by weight, and buffer is present at about 3% by weight. In one embodiment, lactose is present at about 5% by weight, bacteria at about 1.47% by weight, lactose at about 90% by weight, and buffer is present at about 3% by weight. In one embodiment, lactose is present at about 3% by weight, bacteria at about 1.47% by weight, lactose at about 92% by weight, and buffer is present at about 3% by weight. In one embodiment, lactose is present at about 2% by weight, bacteria at about 1.47% by weight, lactose at about 93% by weight, and buffer is present at about 3% by weight. In one embodiment, lactose is present at about 1% by weight, bacteria at about 1.47% by weight, lactose at about 94% by weight, and buffer is present at about 3% by weight. In one embodiment, lactose is present at about 0.5% by weight, bacteria at about 1.47% by weight, lactose at about 95% by weight, and buffer is present at about 3% by weight.

In one embodiment, a therapeutic composition comprises, consists essentially of, or consists of lactose, a probiotic component, a prebiotic component and a buffer component, and is formulated for controlled release of the therapeutic composition. In another embodiment, the therapeutic composition is formulated with an enteric coating. If the bacteria, lactose, buffer and lactose do not make up 100% of the composition by weight, the remaining ingredients can be any pharmaceutically acceptable ingredients intended for consumption by a subject, e.g., human, including, but not limited to, ingredients intended to inhibit clumping and increase pourability, such as silicone dioxide and microcrystalline cellulose, or similar ingredients known in the art. Remaining ingredients can also include ingredients to improve handling, preservatives, antioxidants, flavorings and the like. In one embodiment, a therapeutic composition comprises, consists essentially of, or consists of lactose, a probiotic component, a prebiotic component, and a buffer component in the form of a pill, tablet, caplet, capsule or sachet. In one embodiment the pill, tablet, caplet or capsule is formulated with an enteric coating. In embodiments that include lactose, bacteria (e.g., L. acidophilus), buffer (e.g., calcium phosphate tribasic), microcrystalline cellulose and silicone dioxide, proportions and weights are as shown in the Table I, below. As will be appreciated, weights are merely exemplary, and can be varied. For example, in one embodiment, the weight of lactose is 800 mg (0.8 g) and the other weights can be adjusted accordingly:

TABLE I

| | | | Alternative | |
|---|---|---|---|---|
| | | Percentage | Percentage | Exemplary |
| Ingredients | Weight | Range | Range | Percentage |
| Lactose | 3,200.00 mg | 80-98.5 | 89-94 | 94.01 |
| Buffer, e.g., | 100.00 mg | 0.5-4.0 | 1.2-3.75 | 2.94 |
| Calcium | | | | |
| Phosphate | | | | |
| Tribasic | | | | |
| Bacteria, e.g., | 50.00 mg | 0.25-2.10 | 0.91-1.95 | 1.47 |
| Lactobacillus | | | | |
| Acidophilus | | | | |
| Fructooligo- | 30.00 mg | 0.10-1.89 | 0.40-1.26 | 0.88 |
| sacchrides (FOS) | | | | |
| Handling agent, | 20.00 mg | 0.95-1.15 | 0.18-0.92 | 0.59 |
| e.g., | | | | |
| Microcrystalline | | | | |
| Cellulose | | | | |
| Handling agent, | 4.00 mg | 0.04-0.32 | 0.08-0.19 | 0.12 |
| e.g., Silicon | | | | |
| Dioxide | | | | |

### Additional Ingredients

Additional ingredients include ingredients to improve handling, preservatives, antioxidants, flavorings and the like. For example, in one embodiment, a lactose composition in powdered form can include flavorings such that when mixed in a liquid (e.g., water), the powder can flavor the liquid with various flavors such as grape, strawberry, lime, lemon, chocolate, and the like. In one embodiment, the compositions include microcrystalline cellulose or silicone dioxide. Preservatives can include, for example, benzoic acid, alcohols, for example, ethyl alcohol, and hydroxybenzoates. Antioxidants can include, for example, butylated hydroxyanisole (BHA), butylated hydroxytolulene (BHT), tocopherols (e.g., Vitamin E), and ascorbic acid (Vitamin C).

### General Dosage Forms

Therapeutic compositions comprising lactose described herein include any suitable form, including solid, liquid or powder. In on embodiment a therapeutic composition is provided in a non-liquid form, such as a non-aqueous form. Powdered compositions can be as a powder, contained within capsules, or can be compressed in the form of caplets, tablets, or the like. Powder can be packaged in bulk (e.g., in a container containing sufficient lactose or other substances for a subject to follow for an entire course of treatment with increasing doses of lactose, or a portion of a course of treatment), or as individual packets (e.g., packets containing a single dose of lactose plus other components, or packets containing the dose of lactose and other components needed for a particular day of a lactose treatment regimen). If packaged in bulk, the powder can be in any suitable container, such as a packet, sachet, canister, ampoule, ramekin, or bottle. The container can also include one or more scoops or similar serving devices of a size or sizes appropriate to measure and serve one or more doses of lactose and, optionally, other ingredients included in the powder. Liquid compositions containing lactose and, optionally, other ingredients, can be provided in any suitable liquid, e.g., water or buffer. Liquid compositions can also be provided in a gel or syrup. Liquid compositions can be provided in individual doses as capsules, or prepackaged in single use containers. Liquid compositions can be provided in bulk (e.g., in a container containing sufficient lactose or other substances for one subject in need thereof to follow an entire course of treatment with increasing doses of lactose, or a portion of a course of treatment), or as individual containers, such as cans, bottles, soft packs, and the like (e.g., containers containing a single dose of lactose plus other components in suitable liquid, or containers containing the dose of lactose and other components needed for a particular day of a lactose treatment regimen). The container can also include one or more measuring cups or similar serving devices of a size or sizes appropriate to measure and serve one or more doses of lactose and, optionally, other ingredients included in the liquid.

### Oral Dosage Forms and Components

In one aspect provided herein are methods and compositions formulated for oral delivery to a subject in need thereof. In one embodiment a therapeutic composition comprising lactose is formulated to deliver a composition comprising lactose to a subject in need thereof. In one embodiment a composition is formulated to deliver a composition consisting essentially of lactose to a subject in need thereof. In another embodiment a composition is formulated to deliver a composition comprising lactose and a probiotic to a subject in need thereof. In another embodiment a composition is formulated to deliver a composition comprising lactose and a prebiotic to a subject in need thereof. In another embodiment a composition is formulated to deliver a composition comprising lactose, a probiotic, and a prebiotic to a subject in need thereof.

### Forms

A therapeutic composition comprising lactose is administered in solid, semi-solid, micro-emulsion, gel, or liquid form. Examples of such dosage forms include tablet forms disclosed in U.S. Pat. Nos. 3,048,526, 3,108,046, 4,786,505, 4,919,939, 4,950,484; gel forms disclosed in U.S. Pat. Nos. 4,904,479, 6,482,435, 6,572,871, 5,013,726; capsule forms disclosed in U.S. Pat. Nos. 4,800,083, 4,532,126, 4,935,243, 6,258,380; or liquid forms disclosed in U.S. Pat. Nos. 4,625,494, 4,478,822, 5,610,184; each of which is incorporated herein by reference in its entirety.

A therapeutic composition comprising lactose can be formulated for oral delivery as a tablet, push-fit capsule made of gelatin, as well as soft, sealed capsules made of gelatin and a plasticizer, such as glycerol or sorbitol. Tablets can be made by compression or molding, optionally with one or more accessory ingredients. Compressed tablets can be prepared by compressing in a suitable machine the active ingredient in a free-flowing form such as a powder or granules, optionally mixed with binders (e.g., povidone, gelatin, hydroxypropylmethyl cellulose), inert diluents, preservative, antioxidant, disintegrant (e.g., sodium starch glycolate, cross-linked povidone, cross-linked sodium carboxymethyl cellulose) or lubricating, surface active or dispersing agents. Molded tablets can be made by molding in a suitable machine a mixture of the powdered compound moistened with an inert liquid diluent. The tablets can optionally be coated or scored and can be formulated so as to provide slow or controlled release of the active ingredient therein. Tablets can optionally be provided with an enteric coating, to provide release in parts of the gastrointestinal tract (e.g., the lower intestine) other than the stomach. All formulations for oral administration should be in dosages suitable for such administration. The push-fit capsules can contain the active ingredients in admixture with filler, binders such as starches, and/or lubricants such as talc or magnesium stearate and, optionally, stabilizers. In soft capsules, the active compounds (lactose, prebiotics, or probiotcs) can be dissolved or suspended in suitable liquids, such as fatty oils, liquid paraffin, or liquid polyethylene glycols. In addition, stabilizers can be added. Dragee cores are provided with suitable coatings. For this purpose, concentrated sugar solutions can be used, which can optionally contain gum arabic, talc, polyvinyl pyrrolidone, carbopol gel, polyethylene glycol, or titanium dioxide, lacquer solutions, and suitable organic solvents or solvent mixtures. Dyestuffs or pigments can be added to the tablets or Dragee coatings for identification or to characterize different combinations of active compound doses.

Formulations for oral use can also be presented as a hard gelatin capsule or a soft gelatin capsule. In one embodiment a hard gelatin capsule comprises one or more active ingredients mixed with an inert solid diluent, for example, calcium carbonate, calcium phosphate or kaolin. In another embodiment a soft gelatin capsule comprises one or more active ingredient mixed with a water soluble carrier such as polyethylene glycol or an oil medium, for example peanut oil, liquid paraffin, or olive oil.

Oral liquid preparations can be in the form of, for example, aqueous or oily suspensions, solutions, emulsions syrups or elixirs, or can be presented as a dry product for reconstitution with water or other suitable vehicle before use. Such liquid preparations can contain conventional additives, such as suspending agents, for example sorbitol, methyl cellulose, glucose syrup, gelatin, hydroxyethyl cellulose, carboxymethyl cellulose, aluminum stearate gel or hydrogenated edible fats, emulsifying agents, for example lecithin, sorbitan monooleate, acacia; nonaqueous vehicles (which can include edible oils), for example almond oil, oily esters such as glycerine, propylene glycol, or ethyl alcohol; preservatives, for example methyl or propyl p-hydroxybenzoate or sorbic acid, and, if desired, conventional flavoring or coloring agents.

In one embodiment, a therapeutic composition comprising lactose is provided as a softgel formulation. In one embodiment the softgel comprises a gelatin based shell that surrounds a liquid fill. In another embodiment the softgel comprises a gelatin based shell that surrounds a non-liquid fill. The shell can be made of gelatin, plasticiser (e.g., glycerin and/or sorbitol), modifier, water, color, antioxidant, or flavor. The shell can be made with starch or carrageenan. The outer layer can be enteric coated. In one embodiment, a softgel formulation can include a water or oil soluble fill solution, or suspension of a therapeutic composition, for example, lactose, a probiotic composition, and/or a prebiotic composition, covered by a layer of gelatin.

An enteric coating can control the location of where a therapeutic composition is absorbed in the digestive system. In one embodiment, an enteric coating is designed such that it does not dissolve in the stomach but rather travels to the small intestine, where it dissolves. In one embodiment a therapeutic composition becomes bioavailable when an enteric coating dissolves. An enteric coating can be stable at low pH (such as in the stomach) and can dissolve at higher pH (for example, in the small intestine). Material that can be used in enteric coatings includes, for example, alginic acid, cellulose acetate phthalate, plastics, waxes, shellac, and fatty acids (e.g., stearic acid, palmitic acid). Enteric coatings are described, for example, in U.S. Pat. Nos. 5,225,202, 5,733,575, 6,139,875, 6,420,473, 6,455,052, and 6569457, all of which are herein incorporated by reference in their entireties. The enteric coating can be an aqueous enteric coating. Examples of polymers that can be used in enteric coatings include, for example, shellac (trade name EmCoat 120 N, Marcoat 125); cellulose acetate phthalate (trade name aquacoat CPD®, Sepifilm™ LP, Klucel®, Aquacoat® ECD, and Metolose®); polyvinylacetate phthalate (trade name Sureteric®); and methacrylic acid (trade name Eudragit®).

Enteric coatings can also be used for avoiding irritation of or damage to the mucous membrane of the stomach caused by substances contained in the oral preparation, and for counteracting or preventing formation or release of substances having an unpleasant odor or taste in the stomach. Finally, such coatings can be used for preventing nausea or vomiting on intake of oral preparations.

In one embodiment a lactose composition is provided as a tablet, capsule, or caplet with an enteric coating. In one embodiment the enteric coating is designed to hold the tablet, capsule, or caplet together when in the stomach. The enteric coating can be designed to hold together in acid conditions of the stomach and break down in non-acid conditions and therefore release the drug in the intestines.

In one embodiment, an enteric coated composition comprising or consisting essentially of lactose is administered to a subject. In another embodiment, an enteric coated composition comprising or consisting essentially of lactose and a prebiotic is administered to a subject. In another embodiment, an enteric coated composition comprising or consisting essentially of lactose and a probiotic is administered to a subject. In another embodiment, an enteric coated composition comprising or consisting essentially of lactose, a prebiotic, and a probiotic is administered to a subject. In one embodiment, probiotic bacteria can be administered to a subject using an enteric coating. The stomach has an acidic environment that can kill probiotics. An enteric coating can protect probiotics as they pass through the stomach and small intestine.

In one embodiment enteric coatings can be used to (1) prevent the gastric juice from reacting with or destroying the active substance, (2) prevent dilution of the active substance before it reaches the intestines, (3) ensure that the active substance is not released until after the preparation has passed the stomach, and (4) prevent live bacteria contained in the preparation from being killed because of the low pH-value in the stomach.

In one embodiment a therapeutic composition comprising lactose further comprises an enteric coating. In one embodiment the enteric coating allows for the release of the lactose in the lower intestine of a subject. In one embodiment the enteric coating substantially prevents the digestion of the lactose by the subject before the lactose reaches the lower intestine. In one embodiment the enteric coating substantially prevents the release of the lactose in the stomach or upper intestine of a subject. In one embodiment the enteric coating allows for the release of the lactose in the upper or lower intestine of a subject. In one embodiment the enteric coating allows for the release of the lactose in the lower intestine of a subject. In one embodiment a therapeutic composition comprising lactose is formulated as a tablet with an enteric coating. In another embodiment a therapeutic composition comprising lactose is formulated as a capsule with an enteric coating.

Softgel delivery systems can also incorporate phospholipids or polymers or natural gums to entrap a composition, for example, a prebiotic composition, in the gelatin layer with an outer coating to give desired delayed/control release effects. In one embodiment a softgel capsule is formulated with a fill that has a pH between 2.5-7.5.

A softgel formulation can be sealed tightly in an automatic manner. A softgel formulation can easily be swallowed, allow for product identification using colors and several shapes, allow uniformity, precision and accuracy between dosages, be safe against adulteration, provide good availability and rapid absorption, and offer protection against contamination, light and oxidation. Furthermore, softgel formulations can avoid unpleasant flavors due to content encapsulation.

A softgel formulation comprising a therapeutic composition comprising lactose can be in any of number of different sizes, including, for example, round, oblong, oval, tube, droplet or suppositories.

In one embodiment a therapeutic composition is provided in a dosage form which comprises an effective amount of lactose and one or more release controlling excipients as described herein. Suitable modified release dosage vehicles include, but are not limited to, hydrophilic or hydrophobic matrix devices, water-soluble separating layer coatings, enteric coatings, osmotic devices, multi-particulate devices, and combinations thereof. In one embodiment the dosage form is a tablet, caplet, capsule or lollipop. In another embodiment, the dosage form is a liquid, oral suspension, oral solution, or oral syrup. In yet another embodiment, the dosage form is a gel capsule, soft gelatin capsule, or hard gelatin capsule.

In another embodiment a therapeutic composition comprising lactose is provided in effervescent dosage forms. The compositions can also comprise non-release controlling excipients.

In another embodiment, a therapeutic composition comprising lactose is provided in a dosage form that has at least one component that can facilitate release of the lactose. In a further embodiment the dosage form can be capable of giving a discontinuous release of the compound in the form of at least two consecutive pulses separated in time from 0.1 up to 24 hours. The compositions can comprise one or more release controlling and non-release controlling excipients, such as those excipients suitable for a disruptable semi-permeable membrane and as swellable substances.

In another embodiment a therapeutic composition comprising, consisting essentially of, or consisting of lactose is provided in an enteric coated dosage form. In another embodiment a therapeutic composition comprising, consisting essentially of, or consisting of lactose and a probiotic is provided in an enteric coated dosage form. In another embodiment a therapeutic composition comprising, consisting essentially of, or consisting of lactose and a prebiotic is provided in an enteric coated dosage form. In another embodiment a therapeutic composition comprising, consisting essentially of, or consisting of lactose, a probiotic, and a prebiotic is provided in an enteric coated dosage form. In one embodiment, the prebiotic is FOS. In one embodiment the prebiotic component comprises one or more of GOS, lactulose, raffinose, stachyose, lactosucrose, FOS (i.e. oligofructose or oligofructan), inulin, isomalto-oligosaccharide, xylo-oligosaccharide, paratinose oligosaccharide, transgalactosylated oligosaccharides (i.e. transgalacto-oligosaccharides), transgalactosylate disaccharides, soybean oligosaccharides (i.e. soyoligosaccharides), gentiooligosaccharides, glucooligosaccharides, pecticoligosaccharides, palatinose polycondensates, difructose anhydride III, sorbitol, maltitol, lactitol, polyols, polydextrose, reduced paratinose, cellulose, β-glucose, β-galactose, β-fructose, verbascose, galactinol, and β-glucan, guar gum, pectin, high, sodium alginate, and lambda carrageenan, or mixtures thereof. The composition can also comprise non-release controlling excipients.

In another embodiment a therapeutic composition comprising lactose is provided in a dosage form for oral administration to a subject in need thereof, which comprises one or more pharmaceutically acceptable excipients or carriers, enclosed in an intermediate reactive layer comprising a gastric juice-resistant polymeric layered material partially neutralized with alkali and having cation exchange capacity and a gastric juice-resistant outer layer.

In one embodiment a therapeutic composition comprising lactose is provided in the form of enteric-coated granules, for oral administration. The compositions can further comprise cellulose, disodium hydrogen phosphate, hydroxypropyl cellulose, hypromellose, lactose, mannitol, and sodium lauryl sulfate.

In another embodiment a therapeutic composition comprising lactose is provided in the form of enteric-coated pellets, for oral administration. The compositions can further comprise glyceryl monostearate 40-50, hydroxypropyl cellulose, hypromellose, magnesium stearate, methacrylic acid copolymer type C, polysorbate 80, sugar spheres, talc, and triethyl citrate.

In one embodiment a therapeutic composition comprising lactose is provided in the form of enteric-coated granules, for oral administration. The compositions can further comprise carnauba wax, crospovidone, diacetylated monoglycerides, ethylcellulose, hydroxypropyl cellulose, hypromellose phthalate, magnesium stearate, mannitol, sodium hydroxide, sodium stearyl fumarate, talc, titanium dioxide, and yellow ferric oxide.

In another embodiment a therapeutic composition comprising lactose can further comprise calcium stearate, crospovidone, hydroxypropyl methylcellulose, iron oxide, mannitol, methacrylic acid copolymer, polysorbate 80, povidone, propylene glycol, sodium carbonate, sodium lauryl sulfate, titanium dioxide, and triethyl citrate.

The compositions provided herein can be in unit-dosage forms or multiple-dosage forms. Unit-dosage forms, as used herein, refer to physically discrete units suitable for administration to human or non-human animal subject in need thereof and packaged individually. Each unit-dose can contain a predetermined quantity of an active ingredient(s) sufficient to produce the desired therapeutic effect, in association with other pharmaceutical carriers or excipients. Examples of unit-dosage forms include, but are not limited to, ampoules, syringes, and individually packaged tablets and capsules. Unit-dosage forms can be administered in fractions or multiples thereof. A multiple-dosage form is a plurality of identical unit-dosage forms packaged in a single container, which can be administered in segregated unit-dosage form. Examples of multiple-dosage forms include, but are not limited to, vials, bottles of tablets or capsules, or bottles of pints or gallons. In another embodiment the multiple dosage forms comprise different pharmaceutically active agents. For example a multiple dosage form can be provided which comprises a first dosage element comprising a composition comprising a lactose and a second dosage element comprising prebiotic or a probiotic, which can be in a modified release form.

In this example a pair of dosage elements can make a single unit dosage. In one embodiment a kit is provided comprising multiple unit dosages, wherein each unit comprises a first dosage element comprising a composition comprising a lactose and a second dosage element comprising probiotic, prebiotic or both, which can be in a modified release form. In another embodiment the kit further comprises a set of instructions.

In one embodiment a therapeutic composition comprising lactose can be formulated in various dosage forms for oral administration. In another embodiment a therapeutic composition comprising lactose can also be formulated as a modified release dosage form, including delayed-, extended-, prolonged-, sustained-, pulsatile-, controlled-, targeted-, programmed-release, or intestinal retention dosage forms. These dosage forms can be prepared according to known methods and techniques (see, Remington: The Science and Practice of Pharmacy, supra; Modified-Release Drug Deliver Technology, Rathbone et al., Eds., Drugs and the Pharmaceutical Science, Marcel Dekker, Inc.: New York, N.Y., 2002; Vol. 126, which is herein incorporated by reference in its entirety).

In one embodiment, a therapeutic composition comprising lactose can be administered in a solid or liquid form. Examples of solid dosage forms include but are not limited to discrete units in capsules or tablets, as a powder or granule, or present in a tablet conventionally formed by compression molding. Such compressed tablets can be prepared by compressing in a suitable machine. In one embodiment a tablet comprises lactose and any necessary pharmaceutically acceptable carriers or excipients. In another embodiment a tablet can be optionally coated or scored, having indicia inscribed thereon and can be so formulated as to cause immediate, substantially immediate, slow, controlled or extended release of a composition comprising a prebiotic. In one embodiment the tablet is formulated for controlled release. In one embodiment the tablet is formulated for controlled release in the intestines. In one embodiment the tablet is formulated for controlled release in the lower intestine. In one embodiment the tablet is coated with an enteric coating. Furthermore, dosage forms of the invention can comprise acceptable carriers or salts known in the art, such as those described in the Handbook of Pharmaceutical Excipients, American Pharmaceutical Association (1986), incorporated by reference herein in its entirety.

In one embodiment, an effective amount of a therapeutic composition comprising lactose is mixed with a pharmaceutical excipient to form a solid preformulation composition comprising a homogeneous mixture of compounds described herein. When referring to these compositions as “homogeneous”, it is meant that the lactose and any excipients are dispersed evenly throughout the composition so that the composition can be subdivided into unit dosage forms such as tablets, caplets or capsules. This solid preformulation composition can then subdivided into unit dosage forms of the type described above comprising from, for example, about 20 mg to about 2 g of a therapeutic composition comprising lactose. A therapeutic composition comprising lactose can be formulated, in the case of caplets, capsules or tablets, to be swallowed whole, for example with water.

In another embodiment a therapeutic composition comprising lactose can be in liquid form. The liquid formulations can comprise, for example, an agent in water-in-solution and/or suspension form; and a vehicle comprising polyethoxylated castor oil, alcohol and/or a polyoxyethylated sorbitan mono-oleate with or without flavoring. Each dosage form comprises an effective amount of an active agent and can optionally comprise pharmaceutically inert agents, such as conventional excipients, vehicles, fillers, binders, disintegrants, pH adjusting substances, buffer, solvents, solubilizing agents, sweeteners, coloring agents and any other inactive agents that can be included in pharmaceutical dosage forms for oral administration. Examples of such vehicles and additives can be found in Remington's Pharmaceutical Sciences, 17th edition (1985).

### Manufacturing

The dosage forms described herein can be manufactured using processes that are well known to those of skill in the art. For example, for the manufacture of tablets, an effective amount of lactose can be dispersed uniformly in one or more excipients, for example, using high shear granulation, low shear granulation, fluid bed granulation, or by blending for direct compression. Excipients include diluents, binders, disintegrants, dispersants, lubricants, glidants, stabilizers, surfactants and colorants. Diluents, also termed “fillers”, can be used to increase the bulk of a tablet so that a practical size is provided for compression. Non-limiting examples of diluents include cellulose, microcrystalline cellulose, mannitol, dry starch, hydrolyzed starches, powdered sugar, talc, sodium chloride, silicon dioxide, titanium oxide, dicalcium phosphate dihydrate, calcium sulfate, calcium carbonate, alumina and kaolin. Binders can impart cohesive qualities to a tablet formulation and can be used to help a tablet remain intact after compression. Non-limiting examples of suitable binders include starch (including corn starch and pregelatinized starch), gelatin, sugars (e.g., glucose, dextrose, sucrose, lactose and sorbitol), celluloses, polyethylene glycol, waxes, natural and synthetic gums, e.g., acacia, tragacanth, sodium alginate, and synthetic polymers such as polymethacrylates and polyvinylpyrrolidone. Lubricants can also facilitate tablet manufacture; non-limiting examples thereof include magnesium stearate, calcium stearate, stearic acid, glyceryl behenate, and polyethylene glycol. Disintegrants can facilitate tablet disintegration after administration, and non-limiting examples thereof include starches, alginic acid, crosslinked polymers such as, e.g., crosslinked polyvinylpyrrolidone, croscarmellose sodium, potassium or sodium starch glycolate, clays, celluloses, starches, gums and the like. Non-limiting examples of suitable glidants include silicon dioxide, talc and the like. Stabilizers can inhibit or retard drug decomposition reactions, including oxidative reactions. Surfactants can also include and can be anionic, cationic, amphoteric or nonionic. If desired, the tablets can also comprise nontoxic auxiliary substances such as pH buffering agents, preservatives, e.g., antioxidants, wetting or emulsifying agents, solubilizing agents, coating agents, flavoring agents, and the like.

In one embodiment, a softgel formulation is made with a gelatin mass for the outer shell and a therapeutic composition comprising lactose, optionally including one or more substances (such as a prebiotic, a probiotic or a buffer), inside the shell. In one embodiment, to make a gelatin mass, gelatin powder can be mixed with water and glycerin, heated, and stirred under vacuum. Additives, for example, flavors or colors, can be added to molten gelatin using a turbine mixer and transferred to mobile vessels. The gelatin mass can be kept in a steam-jacketed storage vessel at a constant temperature.

The encapsulation process can begin when the molten gel is pumped to a machine and two thin ribbons of gel are formed on either side of machine. These ribbons can then pass over a series of rollers and over a set of die that determine the size and shapes of capsules. A fill composition, for example a therapeutic composition comprising lactose, optionally including a prebiotic, a probiotic or a buffer can be fed to a positive displacement pump, which can inject it between two gelatin ribbons prior to sealing them together through the application of heat and pressure. To remove excess water, the capsules can pass through a conveyer into tumble dryers where a portion of the water can be removed. The capsules can then be placed on, for example, trays, which can be stacked and transferred into drying rooms. In the drying rooms, dry air can be forced over capsules to remove any excess moisture.

### Release Formulations

Immediate-release formulations of an effective amount of a lactose composition can comprise one or more combinations of excipients that allow for a rapid release of a pharmaceutically active agent (such as from 1 minute to 1 hour after administration). In one embodiment an excipient can be microcrystalline cellulose, sodium carboxymethyl cellulose, sodium starch glycolate, corn starch, colloidal silica, Sodium Laurel Sulphate, Magnesium Stearate, Prosolve SMCC (HD90), croscarmellose Sodium, Crospovidone NF, Avicel PH200, and combinations of such excipients.

“Controlled release” formulations refers to the release of a therapeutic composition comprising lactose from a dosage form at a particular desired point in time after the dosage form is administered to a subject. Controlled release formulations can include one or more excipients, including but not limited to microcrystalline cellulose, sodium carboxymethyl cellulose, sodium starch glycolate, corn starch, colloidal silica, sodium laurel sulphate, magnesium stearate, Prosolve SMCC (HD90), croscarmellose sodium, crospovidone NF, or Avicel PH200. In one embodiment a controlled release formulation provides a sustained but otherwise complete release of a therapeutic composition comprising lactose. In another embodiment a controlled release formulation provides a gastrointestinal location specific release of therapeutic composition comprising lactose. In one embodiment the gastrointestinal location is the upper or lower intestines. In one embodiment the gastrointestinal location is the lower intestine. In another embodiment a controlled release formulation provides a substantially gastrointestinal location specific release of therapeutic composition comprising lactose. In one embodiment the substantially gastrointestinal location specific occurs with the majority of the therapeutic composition comprising lactose released in the upper or lower intestine. In another embodiment the substantially gastrointestinal location specific occurs with the majority of the therapeutic composition comprising lactose released in the lower intestine. In one embodiment the substantially gastrointestinal location specific occurs with 50-100% (such as about 50, 55, 60, 65, 70, 75, 80, 85, 90, 95 or 100%) of the therapeutic composition comprising lactose released in the upper or lower intestine. In another embodiment the substantially gastrointestinal location specific occurs with 50-100% (such as about 50, 55, 60, 65, 70, 75, 80, 85, 90, 95 or 100%) of the therapeutic composition comprising lactose released in the lower intestine. In one embodiment the controlled release formulation comprises an enteric coating.

In one embodiment controlled release can occur at a predetermined time or in a predetermined place within the digestive tract. Examples of controlled release include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,556; 5,871,776; 5,902,632; and 5,837,284 each of which is incorporated herein by reference in its entirety.

In one embodiment a control release dosage form begins its release and continues that release over an extended period of time. Release can occur beginning almost immediately or can be sustained. Release can be constant, can increase or decrease over time, can be pulsed, can be continuous or intermittent, and the like. Generally, however, the release of at least one pharmaceutically active agent from a controlled release dosage form will exceed the amount of time of release of the drug administered as a normal, passive release tablet. Thus, for example, while all the lactose in a uncoated lactose tablet should be released within, for example, four hours after administration, a controlled release dosage form could release a smaller amount of lactose over a period of 6-12 hours, or even longer. Controlled release in accordance with the compositions and methods described herein generally means that the release occurs for a period of six hours or more, such as 12 hours or more.

In one embodiment control release refers to the release of lactose, from a therapeutic composition comprising lactose in which lactose is released according to a desired profile over an extended period of time. In one embodiment, controlled release occurs when there is dissolution of lactose from a formulation of a therapeutic composition comprising lactose within 20-720 minutes after entering the small or large intestine. In another embodiment, controlled release occurs when there is dissolution of lactose from a formulation of a therapeutic composition comprising lactose within 20-720 minutes after entering the large intestine. In another embodiment, controlled release results in substantially complete dissolution after exit from into the stomach. For example, controlled release compositions allow delivery of lactose to a subject in need thereof over an extended period of time. Such release rates can provide therapeutically effective levels of lactose for an extended period of time and thereby provide a longer period of pharmacologic or diagnostic response as compared with conventional dosage forms. When used in connection with the dissolution profiles discussed herein, the term “controlled release” refers to wherein all or less than all of the total amount of lactose in a dosage form, made according to methods and compositions described herein, is delivered to the intestines.

In one embodiment, controlled release refers to controlled release of an agent, from a composition or dosage form in which the agent is released according to a desired profile in which the release occurs after a period of time.

When present in a controlled release oral dosage form, a therapeutic composition comprising lactose described herein can be administered at a substantially lower daily dosage level than conventional release forms.

In one embodiment, the controlled release formulation is capable of releasing about 30 to about 100% of one or more active agents (e.g., lactose, and optionally a prebiotic or a probiotic) contained therein in the intestines of a subject in need thereof following oral administration. In one embodiment, the controlled release formulation is capable of releasing about 30 to about 100% of one or more active agents (e.g., lactose, and optionally a prebiotic or a probiotic) contained therein in the large intestine of a subject in need thereof following oral administration. In another embodiment, the controlled release formulation is capable of releasing about 90% of the one or more active agents (e.g., lactose, and optionally a prebiotic or a probiotic) after entering the intestines following oral administration. In another embodiment, the controlled release layer is capable of releasing about 90% of the one or more active agents (e.g., lactose, and optionally a prebiotic or a probiotic) after entering the large intestine following oral administration.

In one embodiment, the controlled release layer comprises one or more excipients, including but not limited to silicified microcrystalline cellulose (e.g., HD90), croscarmellose sodium (AC-Di-Sol), or magnesium stearate. In one embodiment, the total weight of the controlled release formulation is from about 100 mg to 3 g.

In one embodiment, a controlled release formulation comprises silicified microcrystalline cellulose, hydroxyl methyl propyl cellulose, magnesium stearate, and stearic acid.

Pharmaceutical carriers or vehicles suitable for administration of a therapeutic composition comprising lactose include all such carriers known to those skilled in the art to be suitable for the particular mode of administration. In addition, the compositions can comprise one or more components that do not impair the desired action, or components that supplement the desired action, or have another action.

In another embodiment, an effective amount of a therapeutic composition comprising lactose is formulated in an immediate release form. In this embodiment the immediate-release form can be included in an amount that is effective to shorten the time to its maximum concentration in the blood or digestive tract. By way of example, certain immediate-release pharmaceutical preparations are taught in United States Patent Publication US 2005/0147710A1 entitled, “Powder Compaction and Enrobing,” which is incorporated herein in its entirety by reference.

The dosage forms of a therapeutic composition comprising lactose described herein can also take the form of pharmaceutical particles manufactured by a variety of methods, including but not limited to high-pressure homogenization, wet or dry ball milling, or small particle precipitation (nano spray). Other methods to make a suitable powder formulation are the preparation of a solution of active ingredients and excipients, followed by precipitation, filtration, and pulverization, or followed by removal of the solvent by freeze-drying, followed by pulverization of the powder to the desired particle size.

In one embodiment the particles have a final size of 3-1000 μM, such as at most 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, 1000 μM. In another embodiment the pharmaceutical particles have a final size of 10-500 μM. In one embodiment the pharmaceutical particles have a final size of 50-600 μM. In another embodiment the pharmaceutical particles have a final size of 100-800 μM.

In another embodiment a dosage formulation of a therapeutic composition comprising lactose can be an effervescent dosage form. Effervescent means that the dosage form, when mixed with a liquid such as water or saliva, evolves a gas. Some effervescent agents (or effervescent couple) evolve gas by means of a chemical reaction which takes place upon exposure of the effervescent disintegration agent to water or to saliva in the mouth. This reaction can be the result of the reaction of a soluble acid source and an alkali monocarbonate or carbonate source. The reaction of these two general compounds produces carbon dioxide gas upon contact with water or saliva. An effervescent couple (or the individual acid and base separately) can be coated with a solvent protective or enteric coating to prevent premature reaction. Such a couple can also be mixed with previously lyophilized particles (such as a prebiotic). The acid sources can be any which are safe for human consumption and can generally include food acids, acid and hydrite antacids such as, for example: citric, tartaric, amalic, fumeric, adipic, and succinics. Carbonate sources include dry solid carbonate and bicarbonate salt such as, preferably, sodium bicarbonate, sodium carbonate, potassium bicarbonate and potassium carbonate, magnesium carbonate and the like. Reactants which evolve oxygen or other gasses and which are safe for human consumption are also included. In one embodiment citric acid and sodium bicarbonate is used.

In another embodiment the dosage form can be in a candy form (e.g., matrix), such as a lollipop or lozenge. In one embodiment an effective amount of the lactose is dispersed within a candy matrix. In one embodiment the candy matrix comprises one or more sugars (such as dextrose or sucrose). In another embodiment the candy matrix is a sugar-free matrix. The choice of a particular candy matrix is subject to wide variation. Conventional sweeteners such as sucrose can be utilized, or sugar alcohols suitable for use with diabetic patients, such as sorbitol or mannitol might be employed. Other sweeteners, such as the aspartanes, can also be easily incorporated into a composition in accordance with compositions described herein. The candy base can be very soft and fast dissolving, or can be hard and slower dissolving. Various forms will have advantages in different situations.

A candy mass composition comprising an effective amount of the lactose can be orally administered to a subject in need thereof so that an effective amount of the lactose will be released into the subject's mouth as the candy mass dissolves and is swallowed. A subject in need thereof includes a human adult or child.

In one embodiment a candy mass is prepared that comprises one or more layers which can comprise different amounts or rates of dissolution of the lactose. In one embodiment a multilayer candy mass (such as a lollipop) comprises an outer layer with a concentration of the lactose differing from that of one or more inner layers. Such a drug delivery system has a variety of applications.

The choices of matrix and the concentration of the drug in the matrix can be important factors with respect to the rate of drug uptake. A matrix that dissolves quickly can deliver drug into the subject's mouth for absorption more quickly than a matrix that is slow to dissolve. Similarly, a candy matrix that contains the lactose in a high concentration can release more of the lactose in a given period of time than a candy having a low concentration. In one embodiment a candy matrix such as one disclosed in U.S. Pat. No. 4,671,953 or US Application 2004/0213828 (which are herein incorporated by reference in their entirety) is used to deliver the lactose.

The dosage forms of therapeutic compositions comprising lactose described herein can also take the form of pharmaceutical particles manufactured by a variety of methods, including but not limited to high-pressure homogenization, wet or dry ball milling, or small particle precipitation (e.g., nGimat's NanoSpray). Other methods useful to make a suitable powder formulation are the preparation of a solution of active ingredients and excipients, followed by precipitation, filtration, and pulverization, or followed by removal of the solvent by freeze-drying, followed by pulverization of the powder to the desired particle size. In one embodiment the pharmaceutical particles have a final size of 3-1000 μM, such as at most 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, 1000 μM. In another embodiment the pharmaceutical particles have a final size of 10-500 μM. In another embodiment the pharmaceutical particles have a final size of 50-600 μM. In another embodiment the pharmaceutical particles have a final size of 100-800 μM. In one embodiment a particle comprising lactose is formulated for controlled release of the lactose. In on embodiment the particle is coated with an enteric coating.

In one embodiment an oral dosage form (such as a powder, tablet or capsule) is provided comprising a therapeutic composition comprising about 0.7 gram of lactose, about 0.2 gram of FOS or GOS, about 0.01 g of glucose, about 0.01 g of galactose, about 0.1-0.2 g of a binder, about 0.1-0.2 g of a dispersant, about 0.1-0.2 g of a solubilizer. The oral dosage form can be in the form of a powder, capsule, or tablet. Suitable amounts of binders, dispersants, solubilizers are known in the art for preparation of oral tablets or capsules.

In another embodiment an oral dosage form (such as a powder, tablet or capsule) is provided comprising a lactose composition comprising about 40-99.9% by weight of lactose, about 0.05-2% by weight of a binder, about 0.05-2% by weight of a dispersant, about 0.05-2% by weight of a solubilizer, and optionally about 0.05-5% of a prebiotic or a probiotic.

In another embodiment an oral dosage form (such as a powder, tablet or capsule) is provided a therapeutic composition comprising lactose composition and further comprising about 50, 60, 70, 80, 90, 95, 99.5, 100% by weight of lactose, about 0.05, 0.1, 0.5, 1%, or 2% by weight of a binder, about 0.05, 0.1, 0.5, 1%, or 2% by weight of a dispersant, about 0.05, 0.1, 0.5, 1%, or 2% by weight of a solubilizer, and optionally about 0.05, 0.1, 0.5, 1%, 2%, 3%, 4% or 5% of a prebiotic or a probiotic.

In another embodiment, an oral dosage form is provided a therapeutic composition comprising lactose wherein the oral dosage form is a softgel. In one embodiment the softgel comprises a solid or a liquid suspension or solution form of lactose. In one embodiment, the liquid can comprise about 50%, 55%, 60%, 65%, 70%, 75%, 80%, 85%, 90% 95%, 99% or 99.9% lactose. In one embodiment, the solid can comprise about 50-99.9%, 60-99.9%, 70-99.9%, 80-99.9%, or 90-99.9% lactose. The solid can be, for example, about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.9 or 100% lactose. In one embodiment a soft gel contains a therapeutic composition consisting essentially of lactose. In another embodiment a therapeutic composition comprising lactose can further comprise a prebiotic or a probiotic. In one embodiment a therapeutic composition comprising lactose can further comprise a prebiotic such as FOS or GOS and a probiotic such as a lactobacillus or a bifidobacteria.

In one embodiment, the softgel capsule is about 0.25 mL, 0.5 mL, 1.0 mL, 1.25 mL, 1.5 mL, 1.75 mL, or 2.0 mL. In another embodiment, a softgel capsule comprises about 0.1 g to 3.0 g of a therapeutic composition comprising lactose. In another embodiment, a softgel capsule comprises about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9 or 3.0 g of a therapeutic composition comprising lactose.

In another embodiment, an oral dosage form is provided a therapeutic composition comprising lactose wherein the oral dosage form is a tablet. In one embodiment, the tablet comprises about 50%, 55%, 60%, 65%, 70%, 75%, 80%, 85%, 90% 95%, 99% or 99.9% lactose. In another embodiment the tablet comprises about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.9 or 100% lactose. In one embodiment a tablet contains a therapeutic composition consisting essentially of lactose. In another embodiment a tablet comprises a therapeutic composition comprising lactose that further comprises a prebiotic or a probiotic. In another embodiment a tablet comprising lactose can further comprise a prebiotic such as FOS or GOS and a probiotic such as a lactobacillus or a bifidobacteria.

In another embodiment, a tablet comprises about 0.1 g to 3.0 g of a therapeutic composition comprising lactose. In another embodiment, a tablet comprises about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9 or 3.0 g of a therapeutic composition comprising lactose.

In another embodiment, a therapeutic composition comprising lactose is provided as a suppository for rectal administration.

In another embodiment, a therapeutic composition comprising lactose is formulated as a viscous fluid. In another embodiment, a therapeutic composition comprising lactose is formulated such that its water content is low enough that it does not support microbial growth. In another embodiment, a therapeutic composition comprising lactose is formulated as a viscous fluid without a preservative in a gel capsule. In another embodiment, a therapeutic composition comprising lactose comprises lactose without other prebiotics. In another embodiment, a therapeutic composition comprising lactose comprises lactose and GOS or FOS.

In one embodiment a therapeutic composition comprising lactose is formulated for delivery in a soft gel capsule or a tablet. In one embodiment a therapeutic composition comprising lactose is a high percentage lactose composition, such as a 90-100% lactose composition (e.g., 90, 91, 92, 93, 94, 95, 96, 97, 98, 99 or 100% lactose composition by weight). In another embodiment, a therapeutic composition comprising lactose is formulated such that its water content is low enough that it does not support microbial growth. In another embodiment, a therapeutic composition comprising lactose is formulated as a viscous fluid without a preservative in a gel capsule. In another embodiment, a therapeutic composition comprising lactose is formulated as a viscous fluid without an antioxidant in a gel capsule. In another embodiment the tablet or soft gel capsule comprises about 0.1-3 g of a therapeutic composition comprising lactose.

In another embodiment a therapeutic composition comprising lactose can be formulated as with an oligosaccharide, a foaming component, a water-insoluble dietary fiber, or a neutralizing component. The composition can be in the form of a chewable tablet.

In one embodiment the foaming component can be at least one member selected from the group consisting of sodium hydrogencarbonate, sodium carbonate and calcium carbonate. The neutralizing component can be at least one member selected from the group consisting of citric acid, L-tartaric acid, fumaric acid, L-ascorbic acid, DL-malic acid, acetic acid, lactic acid, and anhydrous citric acid. The water-insoluble dietary fiber can be at least one member selected from the group consisting of crystalline cellulose, wheat bran, oat bran, cone fiber, soy fiber and beet fiber. The formulation can contain a sucrose fatty acid ester, powder sugar, fruit juice powder and/or flavoring material.

In one embodiment a formulation of a therapeutic composition comprising lactose can further comprise additional components selected from various known additives. Such additives include, for example, saccharides (excluding oligosaccharides), sugar alcohols, sweeteners and like excipients, binders, disintegrators, lubricants, thickeners, surfactants, electrolytes, flavorings, coloring agents, pH modifiers, fluidity improvers and the like. Specific examples of the additives include wheat starch, potato starch, corn starch, dextrin and like starches; sucrose, glucose, fructose, maltose, xylose, lactose and like saccharides (excluding oligosaccharides); sorbitol, mannitol, maltitol, xylitol and like sugar alcohols; calcium phosphate, calcium sulfate and like excipients; starch, saccharides, gelatine, gum arabic, dextrin, methyl cellulose, polyvinylpyrrolidone, polyvinyl alcohol, hydroxypropylcellulose, xanthan gum, pectin, gum tragacanth, casein, alginic acid and like binders and thickeners; leucine, isoleucine, L-valine, sugar esters, hardened oils, stearic acid, magnesium stearate, talc, macrogols and like lubricants; CMC, CMC-Na, CMC-Ca and like disintegrators; polysorbate, lecithin and like surfactants; aspartame, alitame and like dipeptides; silicon dioxide and like fluidity improvers; and stevia, saccharin and like sweeteners. The amounts of these additives can be properly selected based on their relation to other components and properties of the preparation, production method, etc.

In one embodiment, a lactose composition can be in a chewable oral dosage formulation. In one embodiment the chewable formulation can comprises between about 40-99.9% lactose. In one embodiment, a lactose composition comprises about 80% lactose, about 5% L-ascorbic acid, about 2% anhydrous citric acid, about 3% sodium hydrogencarbonate, about 3% calcium carbonate, about 2% sucrose fatty acid, about 3% fruit juice powder, and about 2% potassium carbonate. In another embodiment, a lactose composition comprises about 85% lactose, about 5% L-ascorbic acid, about 3% sodium hydrogencarbonate, about 2% sodium carbonate, about 2% sucrose fatty acid ester, about 2% fruit juice powder, and about 1% potassium carbonate. In another embodiment, a lactose composition comprises about 90% lactose, about 2% L-ascorbic acid, about 1% anhydrous citric acid, about 2% sodium hydrogencarbonate, about 2% sodium carbonate, about 2% sucrose fatty acid ester, and about 1% potassium carbonate. In another embodiment, a lactose composition comprises about 95% lactose, about 2% L-ascorbic acid, about 1% sodium hydrogencarbonate, and about 2% fruit juice powder.

### Treatment Regimens

In one embodiment, a treatment regimen to increase a subject's tolerance to lactose is provided. In one embodiment a subject in need thereof is administered a therapeutic composition comprising lactose, optionally further comprising a probiotic component, prebiotic component, a buffer, or a combination thereof is used in combination with other treatments to reduce the symptoms of lactose intolerance. In one embodiment the other treatment for the reduction of symptoms of lactose intolerance is lactase. In one embodiment lactase can be administered before, during, or after treatment with lactose, or any combination thereof. In another embodiment, when symptoms of lactose intolerance are not completely or substantially completely eliminated by lactose treatment, lactase can be administered after prebiotic treatment is terminated. In another embodiment a subject administers the lactase on an as-needed basis.

A subject in need thereof includes a human experiencing one or more symptoms of lactose intolerance. In one embodiment the human is a preterm newborn, a full term newborn, an infant up to one year of age, a young child (e.g., 1 yr to 12 yrs), a teenager, (e.g., 13-19 yrs), an adult (e.g., 20-64 yrs), an elderly adult (65 yrs and older) or a pregnant women.

In one embodiment a treatment regimen of a subject in need thereof lasts for 1-20 days, 1-25 days, 1-30 days, 1-35 days, 1-40 days, 1-45 days, 1-50 days, 5-30 days, 5-35 days, 5-40 days, 5-45 days, 5-50 days, 5-55 days, 5-60 days, 1-60 days, or 5-90 days. In another embodiment a treatment regimen of a subject in need thereof lasts exactly or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days. In one embodiment, a treatment regimen of a subject lasts until a symptom of lactose intolerance decreases.

In one embodiment the amount of lactose provided in a dose of a therapeutic composition comprising lactose as part of a treatment regimen is held constant. For example, a constant dose of lactose can be administered each day to a subject for the duration of the treatment regimens described above. In one embodiment the dosing regimen can be, for example, a constant 2-20 g of lactose per day, or the dosing regimen can be an escalating regimen, for example, 2 g of lactose on day 1 and 20 g of lactose on day 20.

In another embodiment a subject in need thereof can be administered an increasing amount of a therapeutic composition comprising lactose each day. In one embodiment the amount of lactose administered to a subject each day increases by about 0.1 g, 0.2 g, 0.3 g, 0.4 g, 0.5 g, 0.6 g, 0.7 g, 0.8 g, 0.9 g, 1.0 g, 1.1 g, 1.2 g, 1.3 g, 1.4 g, 1.5 g, 1.6 g, 1.7 g, 1.8 g, 1.9 g, 2.0 g, 2.1 g, 2.2 g, 2.3 g, 2.4 g, 2.5 g, 2.6 g, 2.7 g, 2.8 g, 2.9 g, 3.0 g, 3.1 g, 3.2 g, 3.3 g, 3.4 g, 3.5 g, 3.6 g, 3.7 g, 3.8 g, 3.9 g, 4.0 g, 4.1 g, 4.2 g, 4.3 g, 4.4 g, 4.5 g, 4.6 g, 4.7 g, 4.8 g, 4.9 g, or 5.0 g per day. In one embodiment a dosing regimen comprises administering to a subject in need thereof between 0.1 and 20 g or 0.1 and 3 g of lactose per day. The regimen can also include escalating the number of doses per day, for example, 1 dose per day, 2 doses per day, 3 doses per day, 4 doses per day, 5 doses per day, 6 doses per day, 7 doses per day, 8 doses per day, 9 doses per day, or 10 doses per day. For example, 1 dose per day can be administered on day 1, 2 doses per day on day 10, and 3 doses per day on day 20 of a treatment regimen.

In another embodiment, a treatment regimen to increase a subject's tolerance to lactose occurs in phases. A first phase provides a single administration of a therapeutic composition comprising lactose per day. In one embodiment this administration is provided with food (e.g., with breakfast or dinner). The dose of lactose administered can increase over time. For example, the amount of a therapeutic composition comprising lactose administered as part of a first administration to a subject in need thereof increases each day during the first phase. In one embodiment the first phase lasts for a period of about 6 to 18, or about 8 to 16, or about 10 to 14, or about 12 days.

A second phase, following the first phase, provides two administrations of a therapeutic composition comprising lactose per day. The second administration occurs at a different time than the first administration. In one embodiment these two administrations are provided with food, (e.g., with breakfast and dinner). During the second phase the amount of a therapeutic composition comprising lactose administered to a subject in need thereof increases each day. In one embodiment the amount of a therapeutic composition comprising lactose administered as part of a first administration is held constant, while the amount of a therapeutic composition comprising lactose administered as part of a second administration is increased. In one embodiment the second phase lasts for a period of about 6 to 18, or about 8 to 16, or about 10 to 14, or about 12 days.

In one embodiment the second phase is followed by an optional third phase. In the third phase a constant amount of a therapeutic composition comprising lactose is administered twice a day. In one embodiment these two administrations are provided with food, (e.g., with breakfast and dinner). In one embodiment the third phase lasts for another period of about 6 to 18, or about 8 to 16, or about 10 to 14, or about 12 days.

In another embodiment a subject in need thereof is optionally provided a dairy product after the end of the second or third phase. In one embodiment subject in need thereof is provided a dairy product twice a day for 1 to 7 days (e.g., 1, 2, 3, 4, 5, 6, or 7 days). In one embodiment the dairy product is administered with food (e.g., at breakfast or dinner). In another embodiment a therapeutic composition comprising lactose is administered twice a day along with the addition of a dairy product. In one embodiment these two administrations are provided with food, (e.g., with breakfast and dinner). In one embodiment a therapeutic composition comprising lactose is administered twice a day along with the addition of a dairy product for 1, 2, 3, 4, 5, 6 or 7 days (e.g., about 4 days thereafter).

In one embodiment dairy products are not consumed during the first or second phases, (e.g., the first 34 days of the regimen). In one embodiment dairy products are administered for four days after the end of the second phase. In one embodiment the first and second phases, followed by the diary product consumption (e.g., 38 days) comprises the full period in which a therapeutic composition comprising lactose is consumed. Following the end of the treatment regimen a subject can ingest dairy products about every day or two on to maintain the subject's tolerance to lactose. In one embodiment, during the first phase, (e.g., about 18 days), the amount of a therapeutic composition comprising lactose administered to a subject in need thereof increases regularly each day. In one embodiment the amount of a therapeutic composition comprising lactose administered to a subject in need thereof during the second or third phases increases each day for one of the two administrations a day. Optionally, in the final part of the regimen (e.g., the final four days), the amount of a dairy food item, consumed by a subject is increased each day.

In one embodiment a probiotic bacteria can be administered during some or all of the entire period of treatment for a symptom of lactose intolerance. For example, in one embodiment, probiotic bacteria are included in a therapeutic composition comprising lactose that is administered to the subject. In one embodiment, treatment includes one phase in which a composition comprising lactose is administered once per day in conjunction with probiotic bacteria. In one embodiment a subject is administered a therapeutic composition comprising lactose and a probiotic in the first phase of the treatment regime.

In another embodiment the dosing regimen comprises five phases. The first phase comprises administration of a therapeutic composition comprising lactose for about two days, optionally with a probiotic. In the second phase, a therapeutic composition comprising lactose is administered with food once a day (e.g., breakfast, lunch, or dinner) for about 10 to 30 days, or about 14 to 24 days, or about 16 to 20 days, or about 18 days. In the third phase, a therapeutic composition comprising lactose is administered twice a day with food (e.g., both breakfast and dinner) for about 6 to 18 days, or about 8 to 16 days, or about 10 to 14 days, or about 12 days. For the fourth phase lasting another 1, 2, 3, 4, 5, 6 or 7 days (e.g., about 4 days) thereafter, a therapeutic composition comprising lactose is administered with both dinner and breakfast, along with the addition of a lactose containing food (e.g., a dairy product). Prior to the fourth phase, dairy products are not administered (e.g., the first about 30-34 days of the regimen). In one embodiment a treatment regimen lasts 38 days, during which a therapeutic composition comprising lactose is administered for the first 34 days. In one embodiment, after the end of the fourth phase, the regimen optionally includes a fifth phase: the actual ingestion of dairy products every few days to maintain and build up tolerance to lactose, but without the administration of a therapeutic composition comprising lactose, to test the establishment of lactose tolerance. If lactose tolerance is not established, the regimen can be repeated. In the first phase, through the second phase (about 18 days) the amount of a therapeutic composition comprising lactose administered at dinner time increases regularly each day. Thereafter, and in the third period, a second therapeutic composition comprising lactose is administered regularly each day in combination with a breakfast meal. In one embodiment the amount of a second therapeutic composition is increased for each administration during phase 3. In the final days of the fourth phase (e.g., the final four days), a lactose containing food item, such as milk or cheese, is administered each day in increasing amounts.

If an initial treatment regimen is successful in generating lactose tolerance in a lactose intolerant person, and the lactose intolerance recurs, a treatment regimen can be repeated.

In one embodiment, a first dose of a therapeutic composition comprising lactose is administered in increasing amounts for a 6-week period. On the first and second days of this period, a probiotic comprising one or more strains of bacteria, (e.g., a food product comprising live bacteria, such as yoghurt) is administered with a therapeutic composition comprising lactose. Further, during the third phase during this 6-week period, a second dose of a therapeutic composition comprising lactose is administered, typically at breakfast time. An example of the dosing regimen is shown in the following table:

TABLE II

| Week | Day | PM-Dosage | AM-Dosage |
|---|---|---|---|
| 1 | 1 | 1 tbs TC* + 8 oz yogurt | |
| 1 | 2 | 1 tbs TC + 4 oz yogurt | |
| 1 | 3 | 1 tbs TC | |
| 1 | 4 | 2 tbs TC | |
| 1 | 5 | 3 tbs TC | |
| 1 | 6 | 4 tbs TC | |
| 1 | 7 | 5 tbs TC | |
| 2 | 8 | 6 tbs TC | |
| 2 | 9 | 7 tbs TC | |
| 2 | 10 | 8 tbs TC | |
| 2 | 11 | 9 tbs TC | |
| 2 | 12 | 10 tbs TC | |
| 2 | 13 | 11 tbs TC | |
| 2 | 14 | 12 tbs TC | |
| 3 | 15 | 13 tbs TC | |
| 3 | 16 | 14 tbs TC | |
| 3 | 17 | 15 tbs TC | |
| 3 | 18 | 16 tbs TC | |
| 3 | 19 | 16 tbs TC | 1 tbs TC |
| 3 | 20 | 16 tbs TC | 2 tbs TC |
| 3 | 21 | 16 tbs TC | 3 tbs TC |
| 4 | 22 | 16 tbs TC | 4 tbs TC |
| 4 | 23 | 16 tbs TC | 5 tbs TC |
| 4 | 24 | 16 tbs TC | 6 tbs TC |
| 4 | 25 | 16 tbs TC | 7 tbs TC |
| 4 | 26 | 16 tbs TC | 8 tbs TC |
| 4 | 27 | 16 tbs TC | 9 tbs TC |
| 4 | 28 | 16 tbs TC | 10 tbs TC |
| 5 | 29 | 16 tbs TC | 11 tbs TC |
| 5 | 30 | 16 tbs TC | 12 tbs TC |
| 5 | 31 | 16 tbs TC | 13 tbs TC |
| 5 | 32 | 16 tbs TC | 14 tbs TC |
| 5 | 33 | 16 tbs TC | 15 tbs TC |
| 5 | 34 | 16 tbs TC | 16 tbs TC |
| 5 | 35 | 9 oz milk | 9 oz milk |
| 6 | 36 | 10 oz milk | 10 oz milk |
| 6 | 37 | 11 oz milk | 11 oz milk |
| 6 | 38 | 12 oz milk | 12 oz milk |
| 6 | 39 | Cheese 1 oz | |
| 6 | 40 | Cheese 2 oz | |
| 6 | 41 | lactose tolerance | |
| | | achieved | |
| 6 | 42 | lactose tolerance | |
| | | achieved | |

*TC = a therapeutic composition comprising lactose

In one embodiment a therapeutic composition comprising lactose and a probiotic composition are administered to a subject in need thereof as part of a treatment regimen to increase a subject's tolerance to lactose. In one embodiment, in the first day of the regimen, a subject ingests 8 ounces (about 226.4 g) or less of a probiotic composition along with 1 tablespoon (about 14.8 mL) of a therapeutic composition comprising lactose, at the dinner meal. In one embodiment, a subject in need thereof ingests 8 ounces (about 226.4 g) or less of a probiotic composition on the first day, along with 1 tablespoon (about 14.8 mL) of a therapeutic composition comprising lactose with dinner. In one embodiment a subject in need thereof ingests 8 ounces of yogurt comprising live bacteria on the first day, along with 1 tablespoon of milk with dinner (e.g., Table II). On the second day, the amount of the yogurt ingested is reduced by half to 4 ounces (about 113.2 g) or less of a probiotic composition, although the administration of a therapeutic composition comprising lactose remains the same. On the third day, administration of the probiotic composition is stopped, but a therapeutic composition comprising lactose remains at 1 tablespoon (about 14.8 mL). During the fourth through the 18th days, the amount of a therapeutic composition comprising lactose ingested with dinner is increased by 1 tablespoon (about 14.8 mL) each day until 16 tablespoons (about 237 mL) are reached on the day 18.

In the third phase of the regimen, and on, both 1 tablespoon (about 14.8 mL) of a therapeutic composition comprising lactose is ingested in the morning, with breakfast, and 16 tablespoons (about 237 mL) of a therapeutic composition comprising lactose are ingested with dinner. From day 16 until day 34, the same ratio of a therapeutic composition comprising lactose with dinner is maintained, but the morning dose increases daily at a rate of a tablespoon (about 14.8 mL) per day. In this way, by day 34, the subject in need thereof is ingesting 32 tablespoons (about 474 mL) of a therapeutic composition comprising lactose.

On day 35, ingestion of the a therapeutic composition comprising lactose is discontinued and in place thereof, a dairy product such as milk or cheese (without prebiotic composition) is ingested, e.g. 9 ounces (about 255 g) of milk in the morning and an additional 9 ounces (about 255 g) in the evening. The milk amounts are increased incrementally at a rate of an ounce (about 28.3 g) per day, such that, by day 38, the subject is ingesting 12 ounces (about 340 g) of milk with breakfast and an additional 12 ounces (about 340 g) of milk at dinner Optionally, on days 39 through 42, cheese is substituted for milk.

It will be recognized that Table II is only a single exemplary 6-week regimen. In another embodiment the number of days in which a therapeutic composition comprising lactose is administered can vary, and the quantity of the dosages can similarly be modified according to the needs of a particular subject and the symptoms of the subject. Even though there can be variations in both the time period and the dosage rates, the concept of increasing the dosages of a therapeutic composition comprising lactose for specific time periods is maintained and encompassed by the methods herein.

In another embodiment a subject in need thereof can ingest more than 5 tablespoons (about 74 mL) of a therapeutic composition comprising lactose by day 7. As a result, the amount of a therapeutic composition comprising lactose ingested by day 7 can be increased to 6 tablespoons (about 89 mL) on day 8. In one embodiment the amount of a therapeutic composition comprising lactose administered to a subject is increased if the subject does not experience any adverse affects. In another embodiment the amount of a therapeutic composition comprising lactose administered to a subject is not increased if a subject experiences adverse affects.

In another embodiment changes can be made in the regimen in regards to the time intervals between the administration of a therapeutic composition comprising lactose and the first administration of a lactose containing food item (e.g., a dairy product). In one embodiment a subject in need thereof can alter the amount of a therapeutic composition comprising lactose every 12 hours In another embodiment a subject in need thereof can alter the amount of a therapeutic composition comprising lactose every 36 or even 48 hours. A therapeutic composition comprising lactose can be administered in as a powder, a tablet, capsule or a liquid. In one embodiment the therapeutic composition comprising lactose is provided as a powder that can be mixed with water and administered orally. In another embodiment a therapeutic composition comprising lactose can be formulated in one or more capsules, capsules or gels. In another embodiment a therapeutic composition comprising lactose can be supplied in a liquid formulation for oral administration.

In another embodiment a subject in need thereof is treated with a regimen using a therapeutic composition comprising lactose using a dosing schedule as set forth in Table III. In one embodiment a capsule containing a therapeutic composition comprising lactose, is administered to a subject in need thereof. In one embodiment a subject in need thereof completes the protocol on day 34 and optionally challenges the efficacy of his/her lactose tolerance by consuming dairy products, for example as set forth for days 35-40 on Table III. In one embodiment the subject does not require any future treatment for the symptoms of lactose intolerance. In another embodiment, a subject who experiences reoccurrence of or continuing symptoms of lactose intolerance is re-administered a composition comprising lactose.

The table following Table III shows an example of a powder lactose regimen:

TABLE III

| Week | Day | PM-Dosage | AM-Dosage |
|---|---|---|---|
| 1 | 1 | s + 8 oz yogurt | |
| 1 | 2 | s + 4 oz yogurt | |
| 1 | 3 | s | |
| 1 | 4 | m | |
| 1 | 5 | m + s | |
| 1 | 6 | 2 m | |
| 1 | 7 | 2 m + s | |
| 2 | 8 | 3 m | |
| 2 | 9 | 3 m + s | |
| 2 | 10 | 4 m | |
| 2 | 11 | 4 m + s | |
| 2 | 12 | 5 m | |
| 2 | 13 | 5 m + s | |
| 2 | 14 | 6 m | |
| 3 | 15 | 6 m + s | |
| 3 | 16 | 7 m | |
| 3 | 17 | 7 m + s | |
| 3 | 18 | 8 m | |
| 3 | 19 | 8 m | s |
| 3 | 20 | 8 m | m |
| 3 | 21 | 8 m | m + s |
| 4 | 22 | 8 m | 2 m |
| 4 | 23 | 8 m | 2 m + s |
| 4 | 24 | 8 m | 3 m |
| 4 | 25 | 8 m | 3 m + s |
| 4 | 26 | 8 m | 4 m |
| 4 | 27 | 8 m | 4 m + s |
| 4 | 28 | 8 m | 5 m |
| 5 | 29 | 8 m | 5 m + s |
| 5 | 30 | 8 m | 6 m |
| 5 | 31 | 8 m | 6 m + s |
| 5 | 32 | 8 m | 7 m |
| 5 | 33 | 8 m | 7 m + s |
| 5 | 34 | 8 m | 8 m |
| 5 | 35 | 9 oz milk | 9 oz milk |
| 6 | 36 | 10 oz milk | 10 oz milk |
| 6 | 37 | 11 oz milk | 11 oz milk |
| 6 | 38 | 12 oz milk | 12 oz milk |
| 6 | 39 | Cheese 1 oz | |
| 6 | 40 | Cheese 2 oz | |
| 6 | 41 | lactose tolerance | |
| | | achieved | |
| 6 | 42 | lactose tolerance | |
| | | achieved | |

In the foregoing Table III, the designation “s” refers to a single zero sized capsule containing 0.8 gm of lactose, and this is equivalent to about 1 tablespoon of milk. The designation “m” refers to a double-sized zero capsule, which can be filled with 1.6 grams of lactose. In one embodiment the amount of the lactose ingested in any time interval, in accordance with Table III, is substantially identical to that regimen as shown in Table I.

In one embodiment, a composition comprising lactose is administered without a probiotic. In another embodiment, a composition comprising lactose is administered without a prebiotic. In one embodiment a protocol provides for the administration of 0.8 grams of lactose to a subject with food in the PM. On days 2-16, the subject increases the dosage of lactose administered in the PM by 0.8 grams a day. On day 17, the subject starts the same process in the AM, by also administering 0.8 grams in the morning, with food. From day 17-32 the subject continues to orally administer 12.8 grams of lactose in the PM. In one embodiment a subject completes the protocol on day 32. In another embodiment, on day 33, the subject re-introduces dairy products into his/her daily diet. In one embodiment the dairy product is cheese, ice cream, yoghurt or a smoothie, In another embodiment the dairy product is milk. The subject initially administers 6 ounces of a dairy product in the AM and PM, optionally with food. This is slowly increased to 12 ounces a day.

An example of this dosing regimen is shown below in Table IV.

TABLE IV

| | | PM- | AM- | PM- | AM- |
|---|---|---|---|---|---|
| Week | Date | lactose | lactose | Dairy | Dairy |
| 1 | 1 | .8 grams | | | |
| 1 | 2 | 1.6 grams | | | |
| 1 | 3 | 2.4 grams | | | |
| 1 | 4 | 3.2 grams | | | |
| 1 | 5 | 4 grams | | | |
| 1 | 6 | 4.8 grams | | | |
| 1 | 7 | 5.6 grams | | | |
| 2 | 8 | 6.4 grams | | | |
| 2 | 9 | 7.2 grams | | | |
| 2 | 10 | 8 grams | | | |
| 2 | 11 | 8.8 grams | | | |
| 2 | 12 | 9.6 grams | | | |
| 2 | 13 | 10.4 grams | | | |
| 2 | 14 | 11.2 grams | | | |
| 3 | 15 | 12 grams | | | |
| 3 | 16 | 12.8 grams | | | |
| 3 | 17 | 12.8 grams | 0.8 grams | | |
| 3 | 18 | 12.8 grams | 1.6 grams | | |
| 3 | 19 | 12.8 grams | 2.4 grams | | |
| 3 | 20 | 12.8 grams | 3.2 grams | | |
| 3 | 21 | 12.8 grams | 4 grams | | |
| 4 | 22 | 12.8 grams | 4.8 grams | | |
| 4 | 23 | 12.8 grams | 5.6 grams | | |
| 4 | 24 | 12.8 grams | 6.4 grams | | |
| 4 | 25 | 12.8 grams | 7.2 grams | | |
| 4 | 26 | 12.8 grams | 8 grams | | |
| 4 | 27 | 12.8 grams | 8.8 grams | | |
| 4 | 28 | 12.8 grams | 9.6 grams | | |
| 5 | 29 | 12.8 grams | 10.4 grams | | |
| 5 | 30 | 12.8 grams | 11.2 grams | | |
| 5 | 31 | 12.8 grams | 12 grams | | |
| 5 | 32 | 12.8 grams | 12.8 grams | | |
| 5 | 33 | | | 6 oz Milk | 6 oz Milk |
| 5 | 34 | | | 8 oz Milk | 8 oz Milk |
| 5 | 35 | | | 10 oz Milk | 10 oz Milk |
| 6 | 36 | | | 12 oz Milk | 12 oz Milk |

In one embodiment the dosage and timing of administration of a therapeutic composition comprising lactose to a subject can be varied in order to more effectively treat lactose intolerance in a subject in need thereof. For example, the amount and formulation of a therapeutic composition comprising lactose can be varied depending on the characteristics of the subject to which it is administered. Thus, when applying the protocol of the present invention to younger subjects in need thereof, the weight of the subject can be considered. For example, a subject weighing 50 pounds (about 22.5 kg) can be administered smaller amounts of lactose than a subject weighing 15 pounds (about 75 kg). As such, the dose administered to the subject can be proportionally scaled down based on his weight. In one embodiment the order of the doses of a composition comprising lactose can be switched, or can be administered at other times of the day with meals such as lunch or snacks. In one embodiment a dosage of a composition comprising lactose can be administered without food. In another embodiment the program can be modified to shorten or increase the program's length. In another embodiment the program can work with an abbreviated 1 week program or it can be lengthened up to a 10 week program. Although the methods and compositions herein has been described for use in humans, it is also capable of being administered to other mammals.

### Kits

In another aspect, the invention provides kits for the treatment of the symptoms of lactose intolerance. The kits include a therapeutic composition comprising lactose in suitable packaging for use by a subject in need thereof in the treatment of one or more symptoms of lactose intolerance. Any of the compositions described herein can be packaged in the form of a kit. A kit can contain an amount of a therapeutic composition comprising lactose and, optionally, other ingredients as described herein, sufficient for an entire course of treatment, or for a portion of a course of treatment. Thus, in one embodiment, a kit can include sufficient prebiotic (such as GOS or FOS) for the first, second, third, fourth, fifth, and sixth weeks of treatment, or additional weeks of treatment if used, or any combination thereof. Doses of a therapeutic composition comprising lactose can be individually packaged, or the therapeutic composition comprising lactose can be provided in bulk, or combinations thereof. Thus, in one embodiment, a kit provides, in suitable packaging, individual doses of a therapeutic composition comprising lactose that correspond to dosing points in a treatment regimen, wherein the doses are pre-packaged in one or more packages intended for use in the treatment of symptoms of lactose intolerance. For example, a kit can contain doses of a therapeutic composition comprising lactose, as described herein, for a treatment program, where the lactose is administered in increasing doses, so that individual packets of a therapeutic composition comprising lactose contain increasing amounts of lactose in each packet, from lower doses intended for use at the start of the program to higher doses as the program progresses. As doses are provided for later points in the program, two or more doses per day can be provided, each in its individual packet. Each packet can be labeled to indicate the day and time of day that it is intended to be administered, or the packaging containing the packets can be so labeled, or both. A “packet” as used in this context, is any individual container that contains lactose, whether the lactose is in solid or liquid form, and can include a packet that contains powder, tablets, capsules or pills, or a packet that contains a liquid.

In one embodiment, a therapeutic composition comprising lactose can be provided in bulk in a single container, or in two, three, four, five, or more than five containers (e.g., where each container contains enough of a therapeutic composition comprising lactose for a particular week of a treatment program). If more than one bulk container is provided, the bulk containers can be suitably packaged together to provide sufficient lactose for all or a portion of a treatment protocol. The container or containers can be labeled with a label indicating information useful to the subject in need thereof performing the treatment protocol, such as dosing schedules.

A therapeutic composition comprising lactose can further comprise other suitable substances, such as probiotic bacteria, FOS, GOS or buffer, as described herein. The other substance or substances can be packaged separately from the lactose, or mixed with the lactose, or combinations thereof. Thus, in one embodiment, kits of the invention include a powder or liquid containing all the ingredients intended to be used in a course of treatment or a portion of a course of treatment, e.g., lactose and bacteria, FOS, or buffer. In one embodiment, a therapeutic composition comprising lactose is packaged in one package or set of packages, and additional components, such as bacteria, FOS, or buffer, are packaged separately from the lactose.

Kits can further include written materials, such as instructions, expected results, testimonials, explanations, warnings, clinical data, information for health professionals, and the like. In one embodiment, the kits contain a label or other information indicating that the kit is only for use under the direction of a health professional, such as a dietician, nutritionist, nurse, physician, or other appropriate health professional. In another embodiment, the kits contain or include information, such as a label, designating the material within as a medical food.

In one embodiment, the invention provides a kit that includes a container of powder, where the powder includes lactose, and optionally FOS, GOS, pro-biotic bacteria, or a buffer, and a label on the container that indicates proper dosage and schedule of use for the powder. The container can further include scoops or other measuring or serving devices. In one embodiment, the invention provides a kit that includes a container of liquid, where the liquid comprise lactose and additionally FOS, GOS, pro-biotic bacteria, or a buffer, and a label on the container that indicates proper dosage and schedule of use for the liquid. The container can further include measuring or serving devices.

### Business Methods

The invention also provides business methods for marketing compositions and methods for the treatment of the symptoms of lactose intolerance or for overall improvement in gastrointestinal health. In one embodiment, the invention provides a method of doing business that includes marketing a composition for the treatment of symptoms of lactose intolerance wherein the treatment is by administering increasing doses of lactose according to any of the methods described herein, optionally in combination with other substances such as FOS, bacteria, and buffers. In one embodiment, the composition is part of a kit, as described herein. The methods can further include producing such compositions or kits. The marketing can be directly to the consumer, or to suitable health professionals, or combinations thereof. The methods of marketing used in these embodiments of the invention include, but are not limited to, print, television, or radio commercials, infomercials, internet advertising, testimonials, word of mouth, telemarketing, and the like.

Also provided herein is a method of doing business such as providing a therapeutic composition comprising lactose as described herein to another entity that manufactures an already existing brand or product (such as a drink or dairy product) already available to the public. Methods encompass a method of doing business comprising marketing a lactose composition for use with an existing brand or product (drink or dairy product), wherein a therapeutic composition comprising lactose, when combined with the existing brand or product, causes the existing brand or product to have the added beneficial effects of lactose intolerance treatment or improving overall GI health.

### EXAMPLES

### Example 1

A regimen starts with each subject of a group taking 0.8 grams of lactose with dinner each evening. On days 2-16, the dosage of the lactose is increased by 0.8 grams, such that on day 2, the subject takes 1.6 grams, and on day 3, takes 2.4 grams. This process continues until day 16. On day 17, the subject starts the same process with breakfast by consuming 0.8 grams of the product on day 17 and 1.6 grams on day 18. This process continues elevating at the same rate. Simultaneously therewith, the subject is taking 12.8 grams of the lactose-containing product with dinner.

On day 33, each subject starts a reintroduction of diary products into their daily diet. While the diary products can vary, milk is typically the standard product, at least as a starting point. When milk is used, the subject starts with 6 ounces with breakfast and dinner, and gradually increases to 8 ounces, 10 ounces, 12 ounces of milk per day. On day 36, the subject has completed the entire regimen and is able to consume dairy products thereafter with decreased lactose intolerant symptoms. An example of this dosing regimen is shown below in the Table.

| | | PM- | AM- | PM- | AM- |
|---|---|---|---|---|---|
| Week | Date | lactose | lactose | Dairy | Dairy |
| 1 | 1 | .8 grams | | | |
| 1 | 2 | 1.6 grams | | | |
| 1 | 3 | 2.4 grams | | | |
| 1 | 4 | 3.2 grams | | | |
| 1 | 5 | 4 grams | | | |
| 1 | 6 | 4.8 grams | | | |
| 1 | 7 | 5.6 grams | | | |
| 2 | 8 | 6.4 grams | | | |
| 2 | 9 | 7.2 grams | | | |
| 2 | 10 | 8 grams | | | |
| 2 | 11 | 8.8 grams | | | |
| 2 | 12 | 9.6 grams | | | |
| 2 | 13 | 10.4 grams | | | |
| 2 | 14 | 11.2 grams | | | |
| 3 | 15 | 12 grams | | | |
| 3 | 16 | 12.8 grams | | | |
| 3 | 17 | 12.8 grams | .8 grams | | |
| 3 | 18 | 12.8 grams | 1.6 grams | | |
| 3 | 19 | 12.8 grams | 2.4 grams | | |
| 3 | 20 | 12.8 grams | 3.2 grams | | |
| 3 | 21 | 12.8 grams | 4 grams | | |
| 4 | 22 | 12.8 grams | 4.8 grams | | |
| 4 | 23 | 12.8 grams | 5.6 grams | | |
| 4 | 24 | 12.8 grams | 6.4 grams | | |
| 4 | 25 | 12.8 grams | 7.2 grams | | |
| 4 | 26 | 12.8 grams | 8 grams | | |
| 4 | 27 | 12.8 grams | 8.8 grams | | |
| 4 | 28 | 12.8 grams | 9.6 grams | | |
| 5 | 29 | 12.8 grams | 10.4 grams | | |
| 5 | 30 | 12.8 grams | 11.2 grams | | |
| 5 | 31 | 12.8 grams | 12 grams | | |
| 5 | 32 | 12.8 grams | 12.8 grams | | |
| 5 | 33 | | | 6 oz Milk | 6 oz Milk |
| 5 | 34 | | | 8 oz Milk | 8 oz Milk |
| 5 | 35 | | | 10 oz Milk | 10 oz Milk |
| 6 | 36 | | | 12 oz Milk | 12 oz Milk |

In another embodiment the amounts of a therapeutic composition administered to a subject can be varied in the actual times of application. In one embodiment the regimen can be used with the subject starting out at a breakfast time and increasing the dosages on the 17th day at dinner time. In another embodiment a different time of the day can be used. Moreover, the quantities can vary, depending on the physical conditions of the user. Thus, and particularly in the case of children, dosages can be reduced.

### Example 2

A double-blind study of the ability of the lactose-based compositions and methods of the invention was made in order to determine reduction of the symptoms of lactose intolerance. More specifically, the study was conducted to determine whether graduated and controlled administration of a therapeutic composition comprising lactose of the invention to subjects who have been confirmed as having lactose intolerance was effective in order to determine if the regimen of the invention was effective in relieving their lactose intolerant symptoms. For this purpose, a double-blind randomized study was conducted with the subjects following the 38-day regimen with a placebo, or otherwise, the therapeutic composition comprising lactose, itself.

Eight-six persons were pre-screened to determine lactose intolerance. Each of these subjects was between the ages of 18 and 55 and recruited from the Los Angeles area. A pre-Likert scale and a post-Likert scale was used to determine the severity of five particular symptoms of lactose intolerance. Each subject was recruited through advertisements posted in local newspapers in the greater Los Angeles area, as well as the worldwide web. Over 190 subjects were pre-screened for this study. Each subject rated symptoms of bloating, abdominal pain, cramps, diarrhea, and nausea. A ranked scale was used, with 1 indicating no symptoms, 2 indicating slight symptoms, 3 indicating mild symptoms, 4 indicating moderate symptoms, and 5 indicating severe symptoms. The maximum possible score was 20. A score of 14 or higher with no information suggesting milk allergy, irritable bowel symptoms, or pregnancies, allowed each subject to participate.

Each of the subjects were paired by age group and gender. Most members of each pair of subjects began the program within two days. One member of the pair randomly received a supply of the therapeutic composition comprising lactose, while the other received a placebo. This placebo was similar in appearance to the actual therapeutic composition comprising lactose. Detailed instructions for administration were given to each participant. In short, each subject was instructed to take the powdered formula, as well as the powdered placebo, and mix same with water for ingestion. Particular preparation dispensed to each subject was unknown both to the subject and the dispensing individual. A record of each was kept by a third party.

In conducting the study, the regimen described in Example 1 was used. Also, the therapeutic composition comprising lactose of the Table IV of ingredients in the Compositions section, above, was used and particularly that product identified as having the exemplary percentages. Each subject was contacted once a week for the first two weeks, and then each week thereafter, in order to check on their progress. Directions were provided on a personal basis if changes were needed. During the entire program, 5 extra days were included, and each subject was asked to follow this 42-day program until completion. On days 35-37, each subject consumed a measured amount of milk with breakfast and dinner. On day 38, the subjects were asked to incorporate at least 16 ounces of diary product into their diet for the next 5 days. Upon completion, each subject again rated their symptoms using the same Likert scale which was used in the pre-screening procedure. Subjects were again asked for another rating of symptoms after one month of completion of the program.

The data collected from the symptom score sheets was analyzed. Participants provided ratings for five symptoms of lactose intolerance on a 0 (no symptom) to 4 (severe symptom) sale. Data collection was successful. The total symptom scale provided scores ranging from 0 to 20. Data were collected pre-treatment for 73 individuals and 64 individuals (87.7%) completed the program and provided data at the conclusion of that program. Completion rates were 88.9% for those assigned to the group receiving the lactose product and 86.9% for those assigned to the placebo group. 61 individuals provided data between one and two months following completion of the program.

### Summary of Results

Data are summarized for all individuals that provided information for two or more data points (Table, below). At the pre-treatment measurement point, members of the two groups provided statistically equivalent ratings of their symptoms for lactose intolerance (t=0.95, n.s.)1. At the post-treatment measurement point, the group receiving the therapeutic composition comprising lactose provided symptom ratings that, in total, were 54.6 percent lower than their original ratings, while the placebo group ratings declined by 34.1 percent. At post-treatment, analyses (analysis of covariance employing the pre-treatment ratings as the covariate) indicated that the respondents receiving the lactose-containing produced reported a significant decline in symptoms relative to the placebo group (F=8.81, p>0.01). Approximately one month later, participants were contacted again. At that point, the individuals receiving the therapeutic composition comprising lactose provided symptom ratings that were 56.6 percent lower than their original ratings, while the placebo group ratings declined by only 23.3 percent. Again, these results were significantly different (F=18.32, p>0.001):

TABLE V

| | | Pre- | | Post- | | 1-month |
|---|---|---|---|---|---|---|
| | | Treatment | | Treatment | | follow-up |
| | N | Mean (s.d.) | N | Mean (s.d.) | N | Mean (s.d.) |
| Lactose | 32 | 14.1(2.6) | 32 | 3.7(5.6) | 29 | 3.3(5.8) |
| Placebo | 32 | 14.9(3.5) | 32 | 8.1(6.2) | 32 | 10.3(5.6) |

As well, the percentage of respondents with a decline of 10 or more points on the total ratings were examined. As would be expected, the results were similar. For the post-treatment measuring point, 71.9 percent of the Lactose group, but only 37.5 percent of the placebo group reported symptom declines of 10 points or greater on the rating scale (χ²=7.63, p>0.01)². One month later, the observed differences had increased, and 79.3 percent of the Lactose group but only 18.8 percent of the placebo group reported a symptom rating scale decline of at least 10 points (χ²=22.37, p>0.001).

Summary of Pairs with 3 Data Points (−26)

In this section, data are summarized for the 26 matched pairs of individuals that provided information for all 3 data points (Table below). At the pre-treatment measurement point, the two groups were statistically equivalent on their symptom rating scale totals (t=1.19, n.s.). At the post-treatment measurement point, the group receiving the therapeutic composition comprising lactose reported significantly lowered symptom severity relative to the placebo group (t=2.36, p>0.05). In addition, 73.1 percent of the group receiving the therapeutic composition comprising lactose versus 38.5 percent of the placebo group reported a symptom decline of at least 10 points on the rating scale, and this result was also statistically significant (χ²=6.33, p>0.05). At the final measurement point, the group receiving the therapeutic composition comprising lactose provided symptom ratings that, in total, were 54.1 percent lower than their original ratings, while the placebo group ratings declined by only 26.3 percent. Analyses (a matched-pair t-test) indicated that the matched respondents receiving the therapeutic composition comprising lactose reported significantly lowered symptom severity relative to the placebo group (t=3.67, p>0.01). In addition, 76.9 percent of the group receiving the therapeutic composition comprising lactose versus 23.1 percent of the placebo group reported a symptom decline of at least 10 points on the rating scale, and this result was also statistically significant (χ²=15.08, p>0.001).

The summary of the data for 3 data points is set forth in the Table below:

TABLE VI

| | | Pre- | | Post- | | 1-month |
|---|---|---|---|---|---|---|
| | | Treatment | | Treatment | | follow-up |
| | N | Mean (s.d.) | N | Mean (s.d.) | N | Mean (s.d.) |
| Lactose | 26 | 13.8(2.5) | 26 | 3.9(6.0) | 26 | 3.7(6.1) |
| Placebo | 22 | 14.9(3.7) | 26 | 7.9(6.3) | 26 | 9.7(5.7) |

The present study confirmed the occurrence of decrease of symptoms when lactose intolerant subjects ingested a formulated therapeutic composition comprising lactose for 38 days and showed a decrease in the severity of all symptoms when they were challenged with a lactose load (8, 10, and 12 oz glass of milk) after metabolic adaptation compared with pre-adaption severity. Post one month data demonstrated the same degree of improved symptoms as subjects continued to incorporate dairy products into their diets.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

What is claimed is:

1. A composition for increasing lactose tolerance in a subject comprising two or more of lactose, FOS, or Lactobacillus acidophilus, formulated for controlled release.

2. The composition of claim 1, wherein said composition is provided as a capsule or a tablet.

3. The composition of claim 1, wherein said capsule or tablet comprise an enteric coating.

4. The composition of claim 1, wherein said composition comprises about 0.1 to 15 grams of lactose.

5. The composition of claim 1, wherein said composition further comprises a buffer.

6. A method for increasing lactose tolerance in a subject experiencing one or more symptoms of lactose intolerance comprising: administering a composition comprising lactose to the subject for a predetermined number of days, wherein the lactose is formulated for controlled release.

7. The method of claim 6, wherein said composition consists essentially of lactose.

8. The method of claim 6, wherein said composition further comprises a prebiotic.

9. The method of claim 8, wherein said prebiotic further comprises FOS.

10. The method of claim 6, wherein said composition further comprises a probiotic.

11. The method of claim 10, wherein said probiotic comprises Lactobacillus or bifidobacteria.

12. The method of claim 6, wherein said composition further comprises a buffer.

13. The method of claim 6, wherein said controlled release formulation comprises an enteric coating.

14. The method of claim 6, wherein said composition comprising lactose is provided as a powder, a tablet or a capsule.

15. The method of claim 6, wherein said composition comprising lactose is administered without meals.

16. The method of claim 6, wherein said composition comprising lactose is administered in conjunction with meals.

17. The method of claim 6, wherein said one or more symptoms comprise flatulence, heartburn, stomach upset, nausea, bloating, flatulence, diarrhea, abdominal pain, cramping, or vomiting.

18. The method of claim 6, wherein said administration results in reduction of bloating, diarrhea, gastric distention, pain, or flatulence from the consumption of dairy products and other lactose containing compositions.

19. The method of claim 6, wherein after said predetermined number of days, said lactose intolerance remains partially, substantially, or completely eliminated or decreased in severity in a subject for at least about 1 day, 1 week, 1 month, 2 months, 3 months, 4 months, 5 months, 6 months, 9 months, one year, 18 months, two years, three years, four years, or five years.

20. The method of claim 6, wherein said subject has a psychological aversion to dairy products.

21. The method of claim 20, wherein said administering results in a decrease in said psychological aversion to dairy products.

22. The method of claim 20, wherein said subject is an elderly person.

23. The method of claim 20, wherein said subject has osteoporosis.

24. The method of claim 6, wherein said subject has a calcium deficiency.

25. The method of claim 6, wherein said predetermined number of days is until a symptom of lactose intolerance decreases.
